(19) United States
(12) Patent Application Publication
(10) Pub. No.: US 2025/0313569 A1
(43) Pub. Date: Oct. 9, 2025

(54) Pharmaceutical Compounds And Compositions As Modulators Of MAS-Related G-Protein Receptor X2

(71) Applicant: Enanta Pharmaceuticals, Inc., Watertown, MA (US)

(72) Inventors: Xuri GAO, Newtonville, MA (US); Scott A. MITCHELL, Woburn, MA (US); Yat Sun OR, Waltham, MA (US); Xiben LI, Lexington, MA (US); Kevin McGRATH, Brighton, MA (US); Samuel BARTLETT, Brighton, MA (US); Wei LI, Lexington, MA (US); Adam SZYMANIAK, Boston, MA (US)

(21) Appl. No.: 19/173,094

(22) Filed: Apr. 8, 2025

Related U.S. Application Data
(60) Provisional application No. 63/631,623, filed on Apr. 9, 2024.

(51) Int. Cl. C07D 491/107; A61K 31/416; A61K 31/4535; A61K 31/454; A61K 31/4545; A61K 31/4709; A61K 31/4725; A61K 31/497; C07D 211/56; C07D 401/12; C07D 401/14; C07D 405/12; C07D 405/14; C07D 409/12; C07D 409/14; C07D 413/14; C07D 417/14; C07D 471/04

(52) U.S. Cl. CPC C07D 491/107; A61K 31/416; A61K 31/4535; A61K 31/454; A61K 31/4545; A61K 31/4709; A61K 31/4725; A61K 31/497; C07D 211/56; C07D 401/12; C07D 401/14; C07D 405/12; C07D 405/14; C07D 409/12; C07D 409/14; C07D 413/14; C07D 417/14; C07D 471/04

(57) ABSTRACT

The invention provides compounds of formulae (I), or pharmaceutically acceptable salts and pharmaceutical compositions thereof,

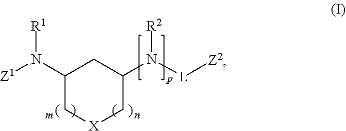

which are useful as modulators of the G-coupled protein receptor MRGPRX2, as well as methods for using such compounds to treat or ameliorate certain diseases or conditions. In some embodiments, the invention provides methods for using such compounds to treat atopic dermatitis, inflammatory disorders, autoimmune disorders, itch-associated conditions, pseudo-allergic reactions, pain-associated conditions and cancer-associated conditions.

### RELATED APPLICATION

This application claims the benefit of U.S. Provisional Application No. 63/631,623, filed on Apr. 9, 2024. The entire teachings of the above application are incorporated herein by reference.

### TECHNICAL FIELD

The present invention relates generally to compounds and pharmaceutical compositions useful as modulators of the Mas-related G-protein receptor X2 (MRGPRX2).

### BACKGROUND OF THE INVENTION

Mas-related G-protein receptors (MRGPRs) are a group of orphan receptors with limited expression in very specialized tissues. Very little is known about the function of most of these receptors. There are eight related receptors in this class expressed in humans, only four of which have readily identifiable orthologs in other species (i.e., MRGPR D, E, F and G). Some of the other four receptors (MRGPR X1, X2, X3 and X4) have counterparts in higher species including dogs and horses, but they do not have a single corresponding ortholog in rodents.

MRGPRX2 corresponds functionally to mouse mrgprb2 and dog MRGPRX2 in mast cells. MRGPRX2 and its ortholog receptors mediate disorders including pseudo-allergic reactions including pseudo-allergic drug reactions, chronic itch (e.g., pruritus), inflammation disorders, pain disorders, skin disorders, wound healing, cardiovascular disease, and lung inflammation/COPD. In one embodiment, both mrgprb2 and MRGPRX2 expression is largely restricted to mast cells. Mast cells are innate immune cells that primarily reside at sites exposed to the external environment, such as the skin, oral/gastrointestinal mucosa and respiratory tract. Mast cells express numerous receptors that respond to mechanical and chemical stimuli. Upon activation, classically by lgE, mast cells release pre-formed mediators from granules (e.g., histamine, proteases, and heparin) and newly synthesized mediators (e.g., thromboxane, prostaglandin D2, leukotriene C4, tumor necrosis factor alpha, cosinophil chemotactic factor, and platelet-activating factor) that elicit allergic and inflammatory responses. Histamine dilates post-capillary venules, activates the endothelium, and increases blood vessel permeability. This causes local edema, warmth, redness, and chemotaxis of other inflammatory cells to the site of release. Histamine also contributes to neuronal sensitization that leads to pain or itch. MRGPRX2 and mrgprb2 mediate immunoglobulin E (lgE) independent activation of mast cells. MRGPRX2 and mrgprb2 are receptors for (or sensitive to activation by) various ligands, including basic secretagogues (small cationic molecules), certain drugs (e.g., cationic peptidergic drugs), neuropeptides, and antimicrobial peptides, and thus are important for non-lgE mediated pseudo-allergic reactions, inflammation, pain, and itch conditions. Mast cells may also contribute to the progression of autoimmune disorders by promoting chronic inflammation in the local tissue microenvironment and ultimately polarizing toward a Th17 immune response. Thus, modulating MRGPRX2 or MRGPRX2 ortholog allows for treatment of autoimmune diseases, pseudo-allergic drug reactions, pain, itch, and inflammatory disorders such as inflammatory bowel disease, urticaria, sinusitis, asthma, rosacea, endometriosis, and other MRGPRX2 or MRGPRX2 ortholog dependent conditions (refer to WO2023192901, WO2022067094, WO2022152852, WO2022152853, WO2022073904, WO2022073905, WO2022067094, WO2022087083, WO2022125636, WO2022140520, WO2021092240, WO2021092262, and WO2021092264).

### SUMMARY OF THE INVENTION

The present invention relates to modulators of MRGPRX2 and to products containing the same. This invention is based on the identification of MRGPRX2 modulator compounds. MRGPRX2 is expressed in mast cells and dorsal root ganglia and is a receptor for (or sensitive to activation by) a diverse group of ligands, including basic secretagogues, certain drugs, neuropeptides, antimicrobial peptides and thus are important in pseudo-allergic reactions, itch, pain, or inflammatory disorders upon exposure.

MRGPRs are sensory receptors that recognize their external environment to exogenous or endogenous signals/chemicals. These receptors therefore likely respond to multiple chemical ligands and/or agonists. MRGPRX2 recognizes signals from agonists such as Substance P, mastoparan, icatibant, ciprofloxacin and atracurium. In certain embodiments, compounds of this invention modulate MRGPRX2 by functioning as inverse agonists that can block multiple chemical entities, and/or as competitive antagonists that can specifically block individual ligands.

The present invention provides compounds represented by Formula (I),

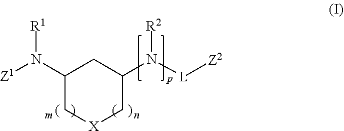

and pharmaceutically acceptable salts, N-oxides, esters, and prodrugs thereof, wherein:
   Z¹ is selected from the group consisting of optionally substituted aryl, optionally substituted heteroaryl, optionally substituted —C₁-C₈ alkyl, optionally substituted —C₁-C₈ alkoxy, optionally substituted —C₃-C₁₂ cycloalkyl, and optionally substituted 3- to 12-membered heterocycloalkyl;
   R¹ is selected from the group consisting of hydrogen, optionally substituted —C₁-C₆ alkyl, optionally substituted —C₃-C₈ cycloalkyl, optionally substituted 3- to 8-membered heterocycloalkyl, —C(O)R¹¹, —C(O)OR¹¹, —C(O)N(R¹²)(R¹³) and —S(O)₂R¹¹;
   R¹¹ is selected from the group consisting of optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted aryl, optionally substituted arylalkyl, optionally substituted heteroarylalkyl, and optionally substituted heteroaryl;
   R¹² and R¹³ are each independently selected from the group consisting of hydrogen, optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted aryl, optionally substituted arylalkyl, optionally substituted heteroarylalkyl, and optionally substituted heteroaryl; alternatively, R¹² and R¹³ are taken together with the nitrogen atom to which they are attached to form an optionally substituted 3-8 membered heterocyclic containing 0, 1, 2, or 3 double bonds;
   alternatively, R¹ and Z¹ are taken together with the nitrogen atom to which they are attached to form an optionally substituted heterocyclic or an optionally substituted heteroaryl;
   R² is selected from the group consisting of hydrogen, optionally substituted —C₁-C₆ alkyl, optionally substituted —C₃-C₈ cycloalkyl;
   p is 0 or 1;
   L is —C(O)—, —S(O)₂—, or absent;
   Z² is selected from the group consisting of:
   1) Optionally substituted aryl;
   2) Optionally substituted heteroaryl;
   3) Optionally substituted —C₃-C₁₂ cycloalkyl;
   4) Optionally substituted —C₃-C₁₂ cycloalkenyl;
   5) Optionally substituted 3- to 12-membered heterocycloalkyl;
   6) Optionally substituted —C₁-C₈ alkyl;
   7) Optionally substituted arylalkyl;
   8) Optionally substituted heteroarylalkyl;
   9) —N(R¹²)(R¹³); and
   10) —OR¹¹;
   m is 1 or 2;
   n is 1 or 2;
   X is —NR³—, —O—, —S—, —S(O)—, or —S(O)₂—;
   R³ is selected from the group consisting of:
   1) Hydrogen;
   2) Optionally substituted —C₁-C₈ alkyl;
   3) Optionally substituted —C₃-C₈ cycloalkyl;
   4) Optionally substituted 3- to 8-membered heterocycloalkyl;
   5) Optionally substituted aryl;
   6) Optionally substituted heteroaryl;
   7) Optionally substituted arylalkyl;
   8) Optionally substituted heteroarylalkyl;
   9) —C(O)F;
   10) —C(O)R¹¹;
11) —C(O)OR¹¹;
12) —C(O)N(R¹²)(R¹³); and
13) —S(O)₂R¹¹.

### DETAILED DESCRIPTION OF THE INVENTION

In one embodiment of the present invention is a compound of Formula (I) described above, or a pharmaceutically acceptable salt thereof.

In certain embodiments of the compounds of Formula (I), R¹ is hydrogen or optionally substituted methyl.

In certain embodiments of the compounds of Formula (I), R² is hydrogen or optionally substituted methyl.

In certain embodiments of the compounds of Formula (I), R¹ is hydrogen and R² is hydrogen.

In certain embodiments of the compounds of Formula (I), X is —N(R³)—.

In certain embodiments of the compounds of Formula (I), X is —NC(O)(R¹¹)—.

In certain embodiments of the compounds of Formula (I), X is —N(Ac)—, —S(O)₂—, or —O—. In certain embodiments X is —N(Ac)—.

In certain embodiments of the compounds of Formula (I), L is —C(O)— or —S(O)₂—. In certain embodiments L is —C(O)—.

In certain embodiments of the compounds of Formula (I), m is 1 and n is 1.

In certain embodiments of the compounds of Formula (I), p is 1.

In certain embodiments of the compounds of Formula (I), m is 1, nis 1, and p is 1.

In certain embodiments of the compounds of Formula (I), Z¹ is derived from one of the following by removal of a hydrogen atom and is optionally substituted:

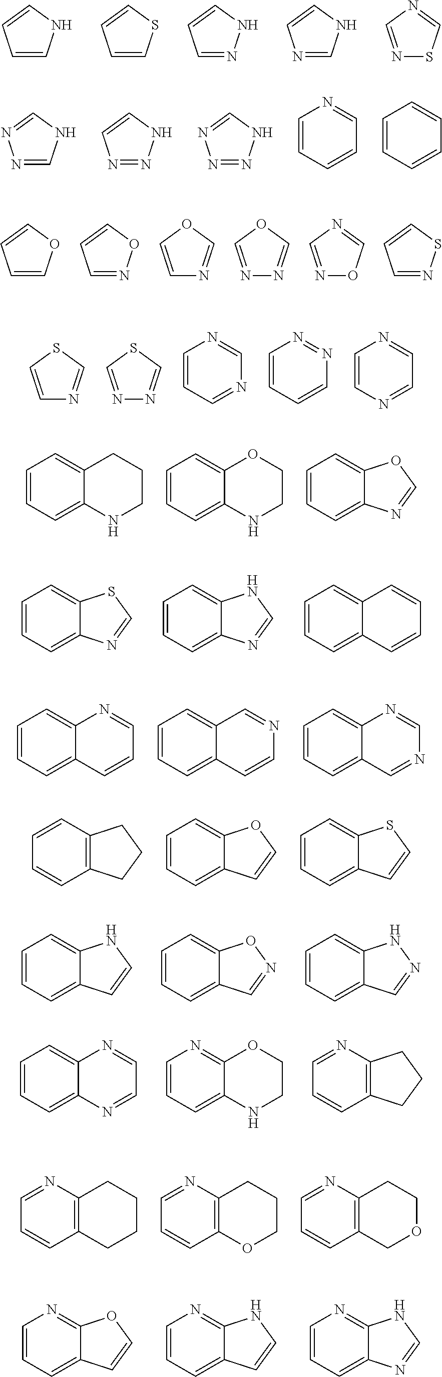

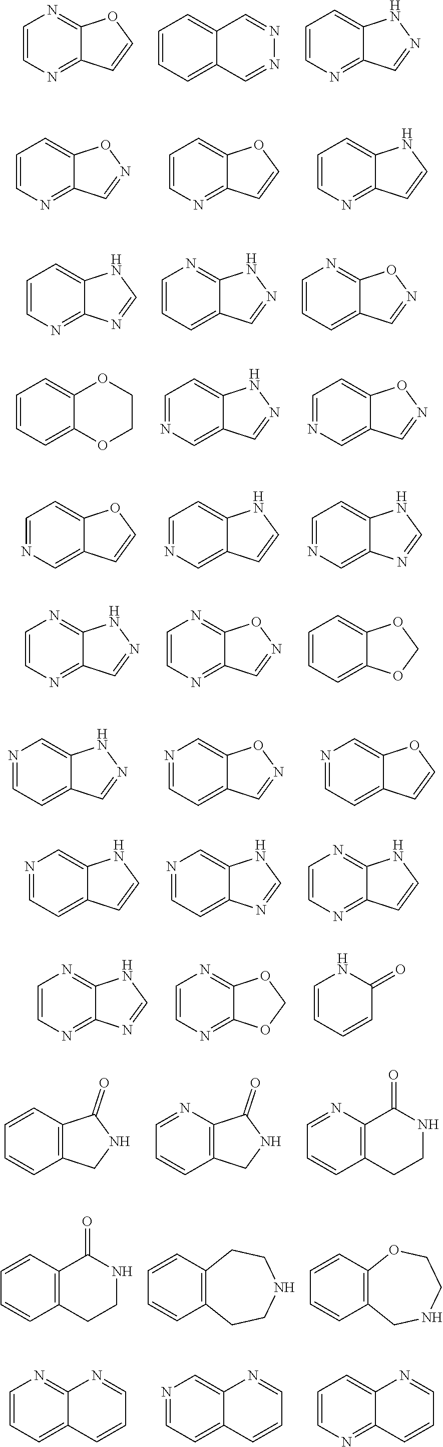

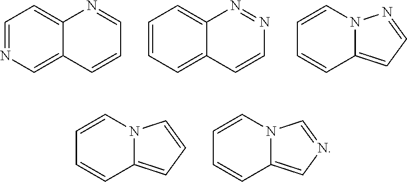

In certain embodiments of the compounds of Formula (I), Z¹ is quinolyl, phenyl, imidazopyridyl, benzothienyl, pyridyl or thiazolyl, each of which is optionally substituted. In certain embodiments Z¹ is optionally substituted phenyl or is selected from the groups below:

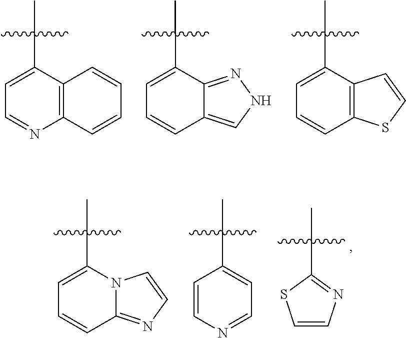

each of which is optionally substituted.

In certain embodiments of the compounds of Formula (I), Z² is derived from one of the following by removal of a hydrogen atom and is optionally substituted:

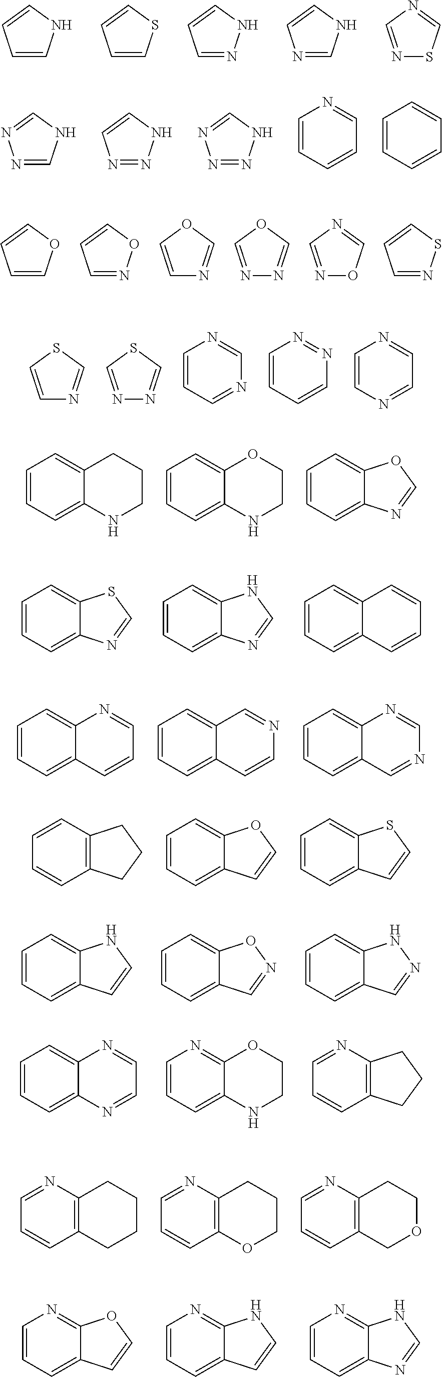

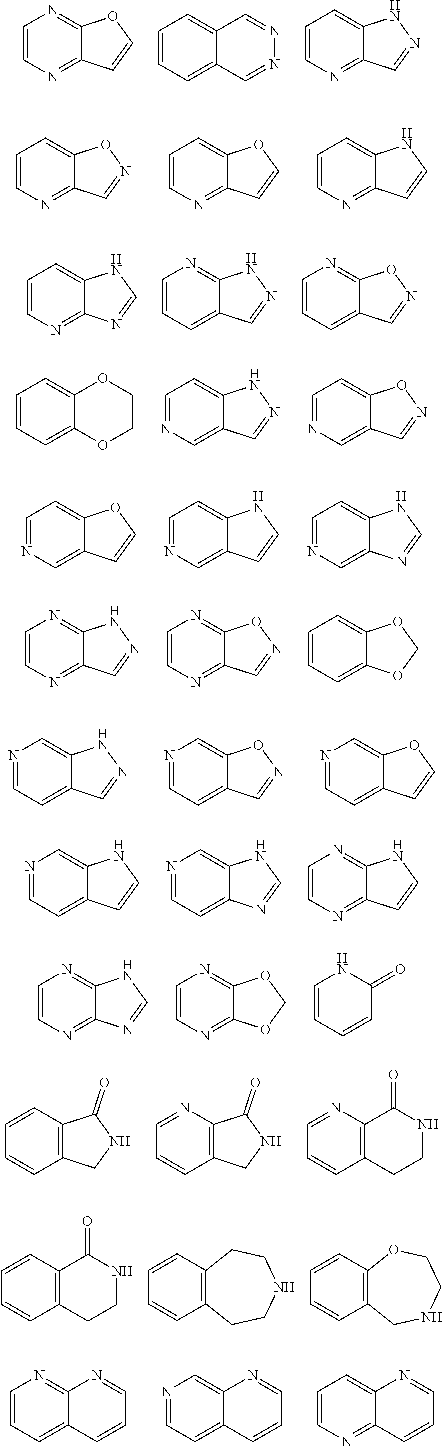

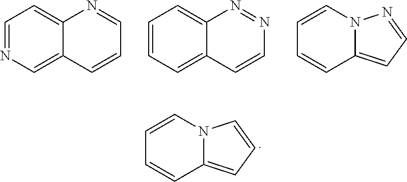

In certain embodiments of the compounds of Formula (I). Z² is derived from one of the following by removal of a hydrogen atom and is optionally substituted:

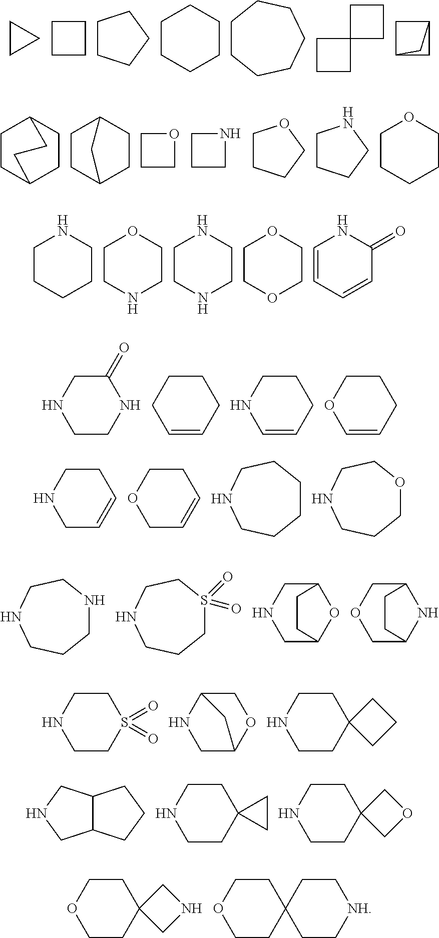

In certain embodiments of the compounds of Formula (I), Z² is phenyl, pyrazolyl, triazolyl, or indazolyl, each of which is optionally substituted.

In certain embodiments of the compounds of Formula (I), Z² is phenyl substituted by one or two substituents independently selected from halo, preferably fluoro; C₁-C₄-alkoxy, preferably methoxy; —NHSO₂—C₁-C₄-alkyl, preferably —NHSO₂CH₃; —CN; difluoromethyl; 2-hydroxyisopropyl. In certain embodiments the number of substituents in one. In certain embodiments Z² is phenyl substituted at the 3 position with —NHSO₂CH₃.

In certain embodiments of the compounds of Formula (I), Z² is optionally substituted C₁-C₈ alkyl.

In certain embodiments of the compounds of Formula (I), Z² is optionally substituted —C₁-C₈ haloalkyl.

In certain embodiments, the compound of Formula (I) is represented by Formula (II):

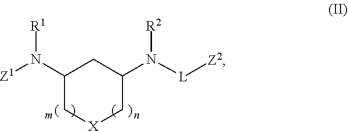

wherein R¹, R², Z¹, Z², X, L, m and n are as previously defined.

In a preferred embodiment, a compound of the invention is represented by Formula (IIa):

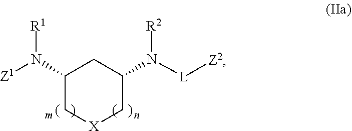

wherein R¹, R², Z¹, Z², X, L, m and n are as previously defined.

In certain embodiments, the compound of Formula (I) is represented by one of Formulae (III-1)˜(III-2):

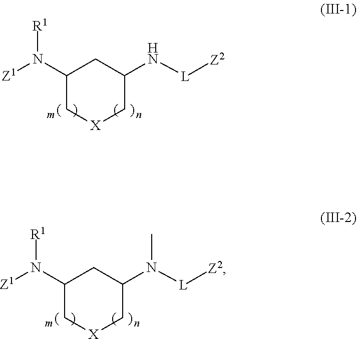

wherein R¹, Z¹, Z³, X, L, m and n are as previously defined.

In certain embodiments, the compound of Formula (I) is represented by one of Formulae (IV-1)˜(IV-4):

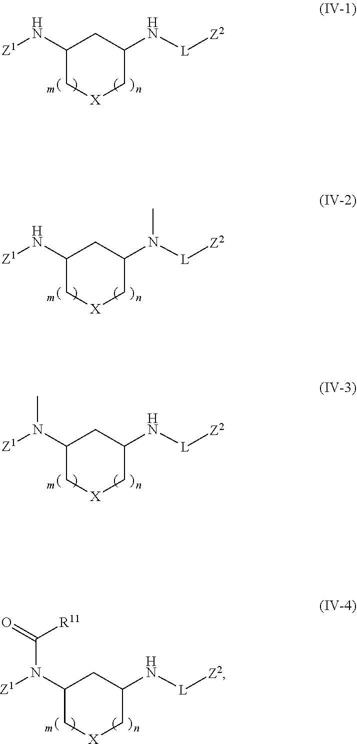

wherein R¹¹, Z¹, Z², X, L, m and n are as previously defined.

In certain embodiments, the compound of Formula (I) is represented by one of Formulae (V-1)˜(V-4):

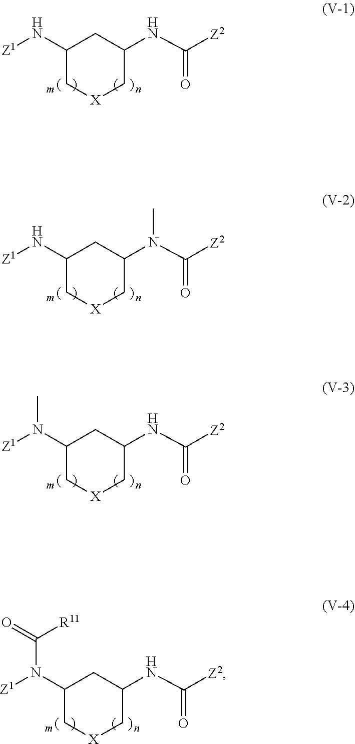

wherein R¹¹, Z¹, Z², X, m and n are as previously defined.

In a preferred embodiment, the compound of Formula (I) is represented by one of Formulae (V-1a)˜(V-4a):

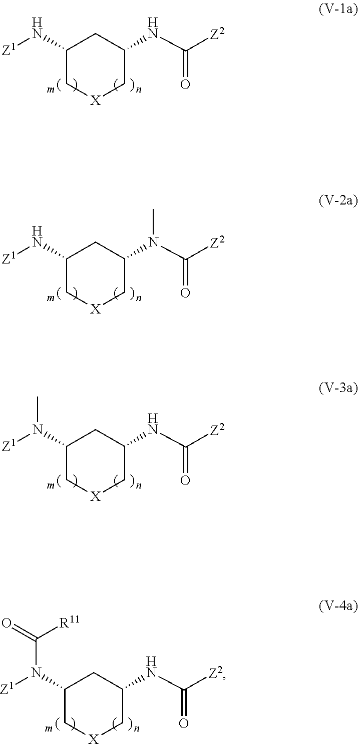

wherein R¹¹, Z¹, Z², X, m and n are as previously defined.

In certain embodiments, the compound of Formula (I) is represented by one of Formulae (VI-1)˜(VI-3):

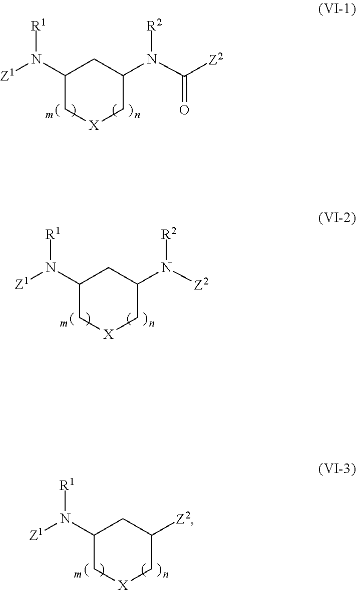

wherein R¹, R², Z¹, Z², X, m and n are as previously defined.

In certain embodiments, the compound of Formula (I) is represented by one of Formulae (VII-1)˜(VII-3):

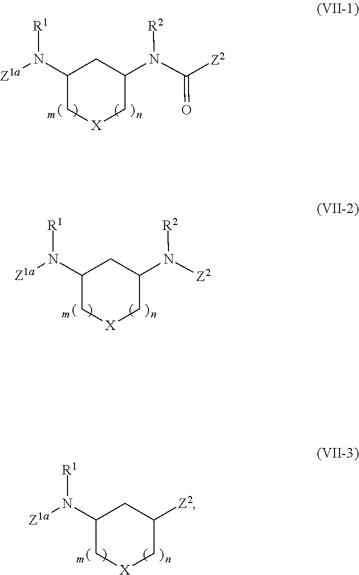

wherein Z^{1a} is Z¹, where Z¹ is as previously defined. Preferably, Z^{1a} is optionally substituted —C₃-C₁₂ cycloalkyl, optionally substituted 3- to 12-membered heterocycloalkyl, optionally substituted aryl, or optionally substituted heteroaryl; R¹, R², Z², X, m and n are as previously defined.

In certain embodiments, the compound of Formula (I) is represented by one of Formulae (VIII-1)˜(VIII-3):

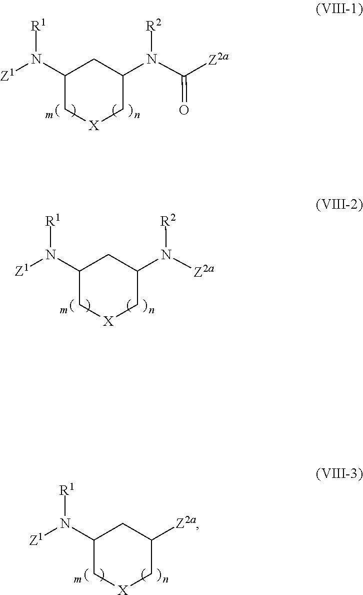

wherein Z^{2a} is Z², where Z² is as previously defined. Preferably Z^{2a} is optionally substituted —C₃-C₁₂ cycloalkyl, optionally substituted 3- to 12-membered heterocycloalkyl, optionally substituted aryl, or optionally substituted heteroaryl, R¹, R², Z¹, X, m and n are as previously defined.

In certain embodiments, the compound of Formula (I) is represented by one of Formulae (IX-1)˜(IX-3):

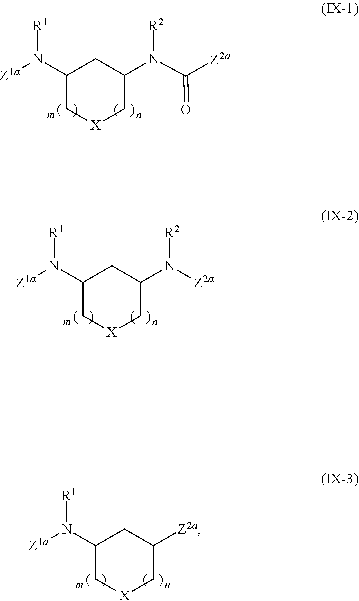

wherein R¹, R², Z^{1a}, Z^{2a}, X, m and n are as previously defined.

In certain embodiments, the compound of Formula (I) is represented by one of Formulae (X-1)˜(X-9):

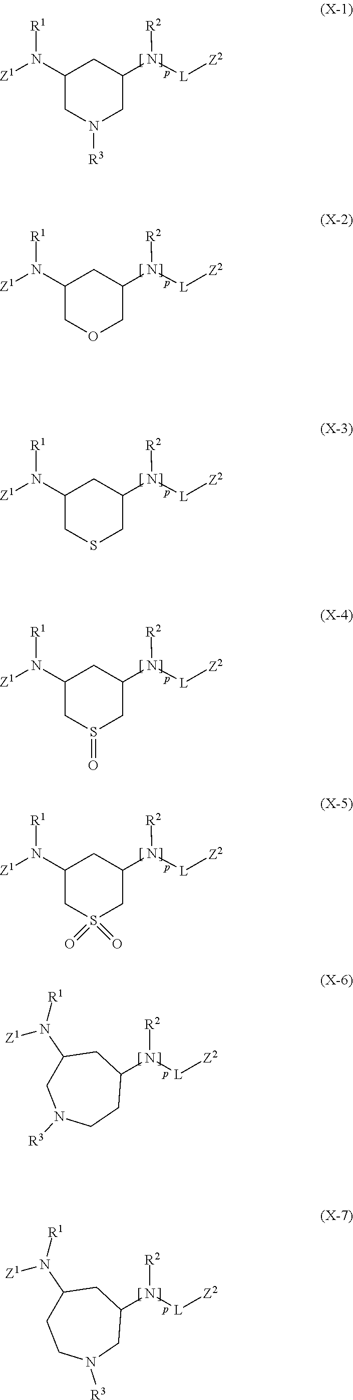

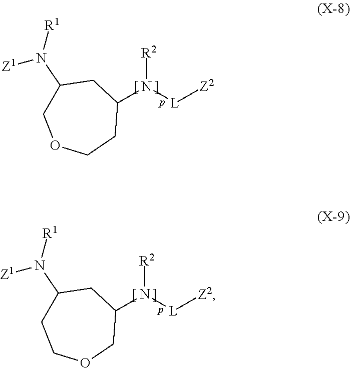

wherein R¹, R², R³, Z¹, Z², L, and p are as previously defined.

In certain embodiments, the compound of Formula (I) is represented by one of Formulae (XI-1)˜(XI-9):

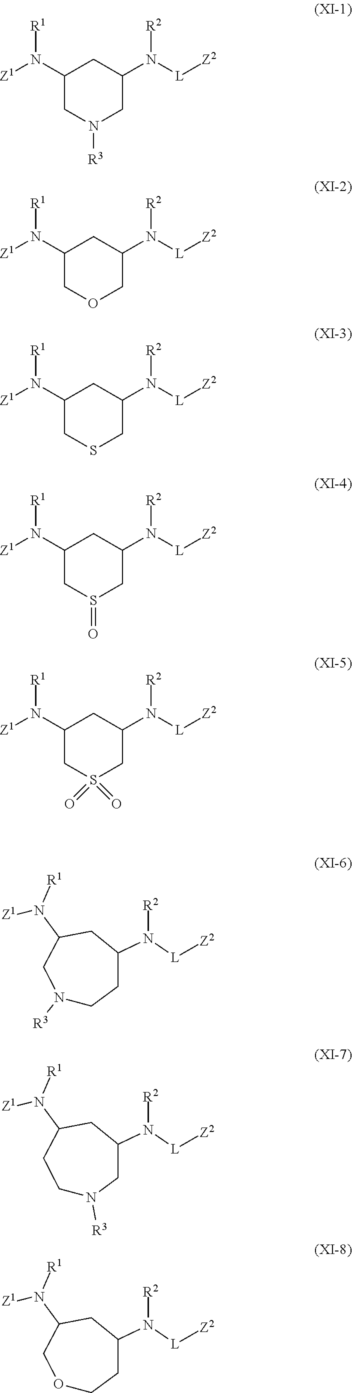

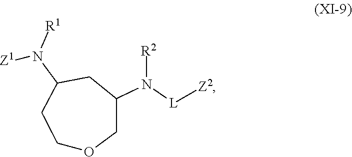

wherein R¹, R², R³, L, Z¹ and Z² are as previously defined.

In certain embodiments, the compound of Formula (I) is represented by one of Formulae (XII-1)˜(XII-9):

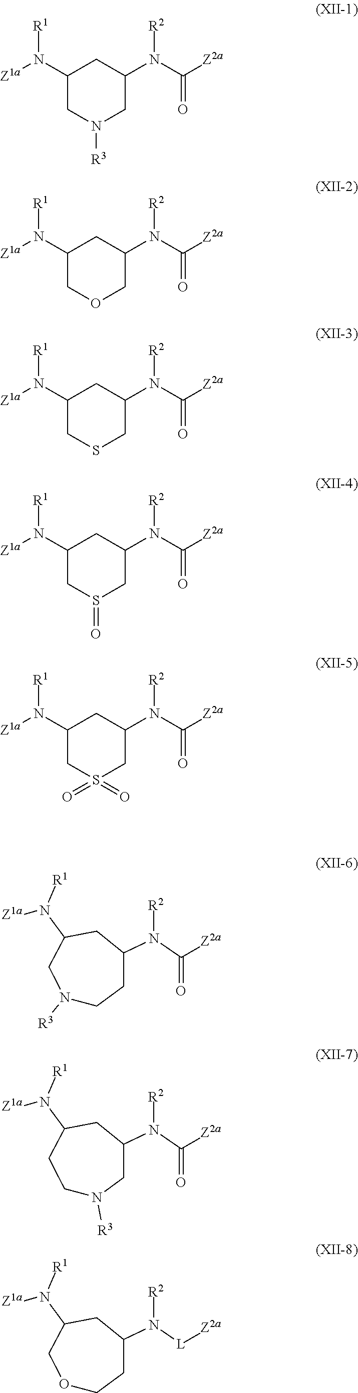

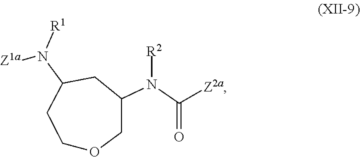

wherein Z^{1a}, Z^{2a}, R¹, R² and R³ are as previously defined.

In certain embodiments, the compound of Formula (I) is represented by one of Formulae (XIII-1)˜(XIII-9):

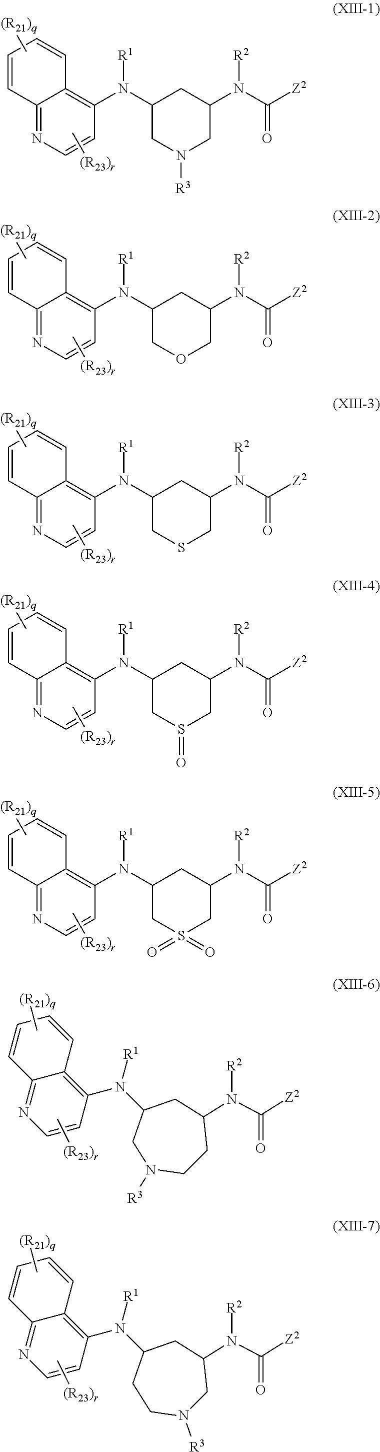

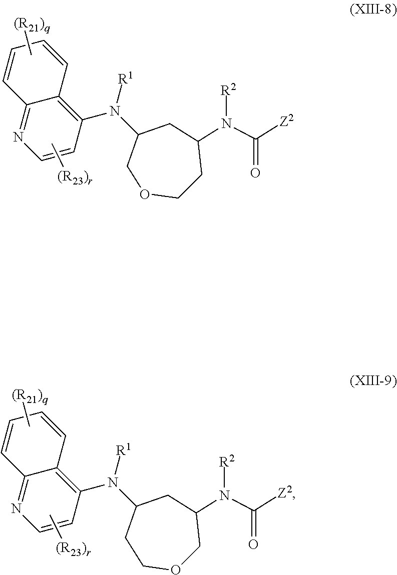

wherein q is 0, 1, 2, 3, or 4 and r is 0, 1 or 2;
each R₂₁ and R₂₃ is independently selected from the group consisting of:
   1) halogen;
   2) —CN;
   3) —OH;
   4) —OR¹¹;
   5) —NR¹²R¹³;
   6) Optionally substituted —C₁-C₈ alkyl;
   7) Optionally substituted —C₁-C₈ haloalkyl;
   8) Optionally substituted —C₃-C₈ cycloalkyl;
   9) Optionally substituted 3- to 8-membered heterocycloalkyl;
   10) Optionally substituted aryl; and
   11) Optionally substituted heteroaryl;
and R¹¹, R¹², R¹³, Z², R¹, R² and R³ are as previously defined.

In certain embodiments, the compound of Formula (I) is represented by one of Formulae (XIV-1)˜(XIV-9):

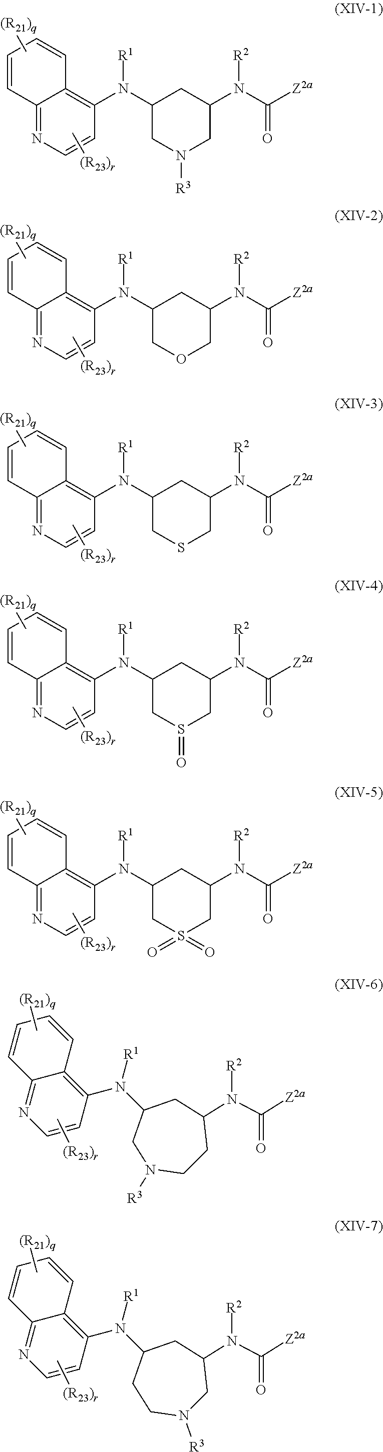

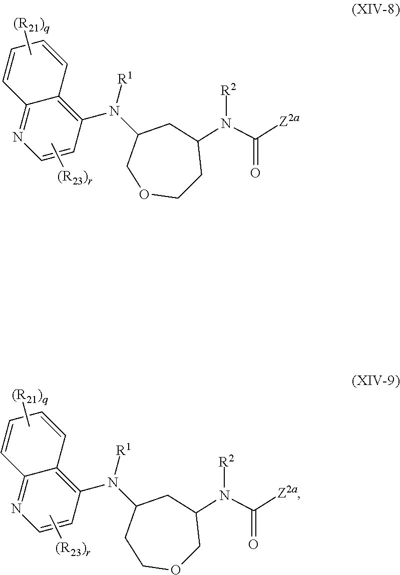

wherein Z^{2a}, R¹, R², R³, R₂₁, R₂₃, r, and q are as previously defined.

In certain embodiments, the compound of Formula (I) is represented by one of Formulae (XV-1)˜(XV-3):

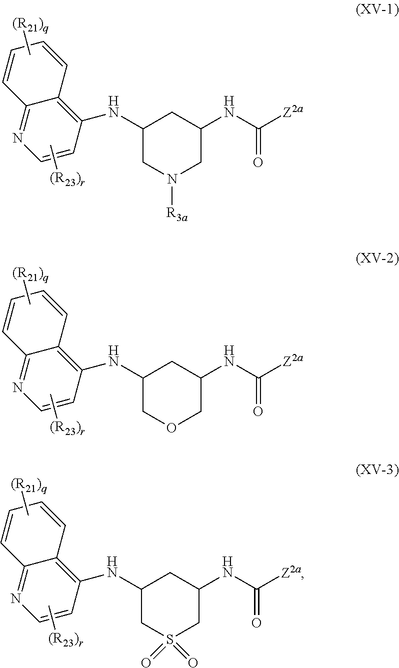

wherein R₃ₐ is selected from the group consisting of:
   1) Hydrogen;
   2) —C(O)R¹¹;
   3) —C(O)₂R¹¹;
   4) —SO₂R¹¹;
   5) Optionally substituted —C₁-C₈alkyl;
   6) Optionally substituted 3- to 8-membered heterocycloalkyl;
   7) Optionally substituted aryl; and
   8) Optionally substituted heteroaryl;
Z^{2a}, R¹¹, R₂₁, R₂₃, r, and q are as previously defined.

In a preferred embodiment, the compound of Formula (I) is represented by one of Formulae (XV-1a)˜(XV-3a):

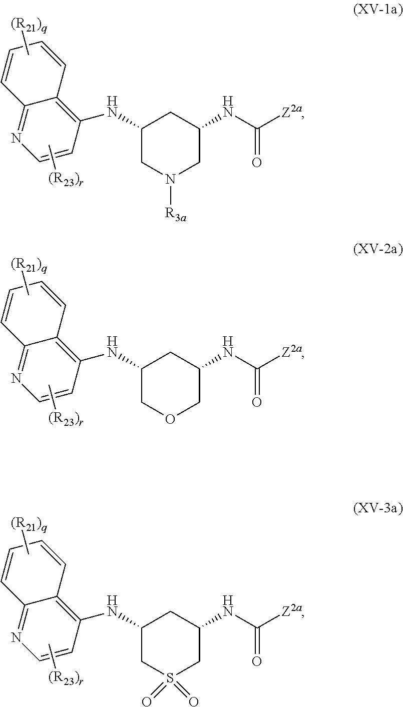

wherein Z^{2a}, R₃ₐ, R₂₁, R₂₃, r, and q are as previously defined.

In certain embodiments, the compound of Formula (I) is represented by one of Formulae (XV-4)˜(XV-6):

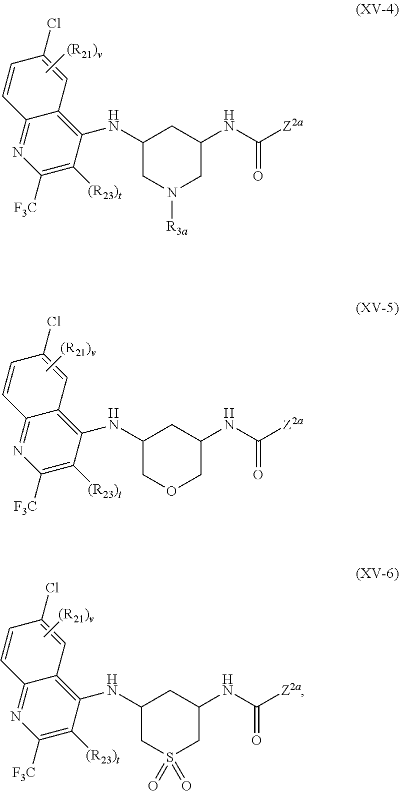

wherein t is 0 or 1; v is 0, or 1; Z^{2a}, R₃ₐ, R₂₁, and R₂₃ are as previously defined.

In certain embodiments, the compound of Formula (I) is represented by one of Formulae (XV-4a)˜(XV-6a):

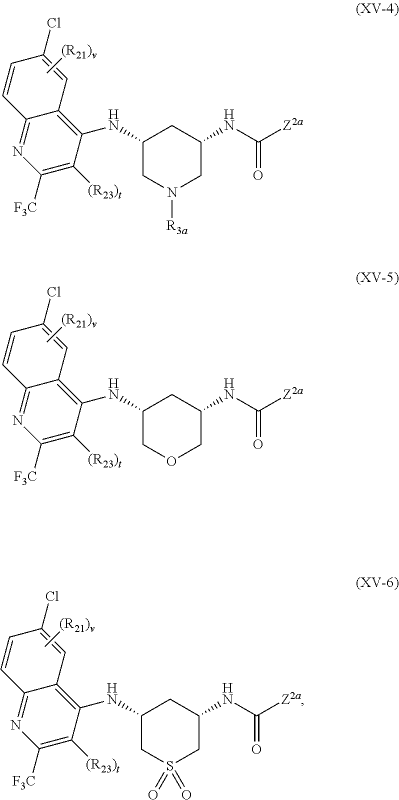

wherein t, v, Z^{2a}, R₃ₐ, R₂₁, and R₂₃ are as previously defined.

In certain embodiments, the compound of Formula (I) is represented by one of Formulae (XVI-1)˜(XVI-3):

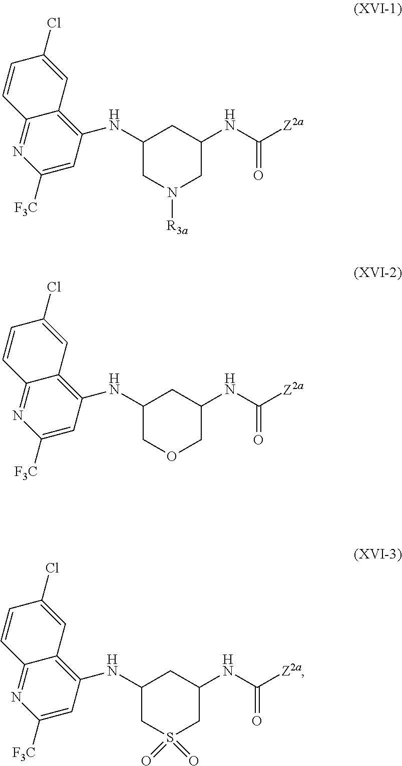

wherein Z^{2a} and R₃ₐ are as previously defined.

In a preferred embodiment, the compound of Formula (I) is represented by one of Formulae (XVI-1a)˜(XVI-3a):

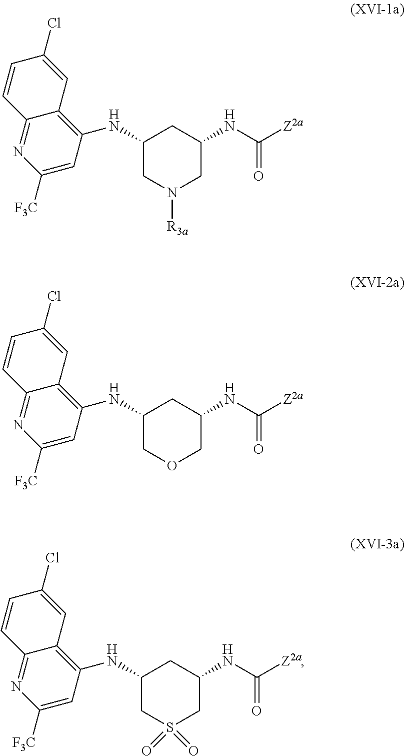

wherein Z^{2a} and R₃ₐ are as previously defined.

In the embodiments, the compound of Formula (I) is represented by one of Formulae (XVI-1)˜(XVI-3), or one of Formulae (XVI-1a)˜(XVI-3a), wherein Z^{2a} is selected from one of the following by removal of a hydrogen atom and Z^{2a} is optionally substituted:

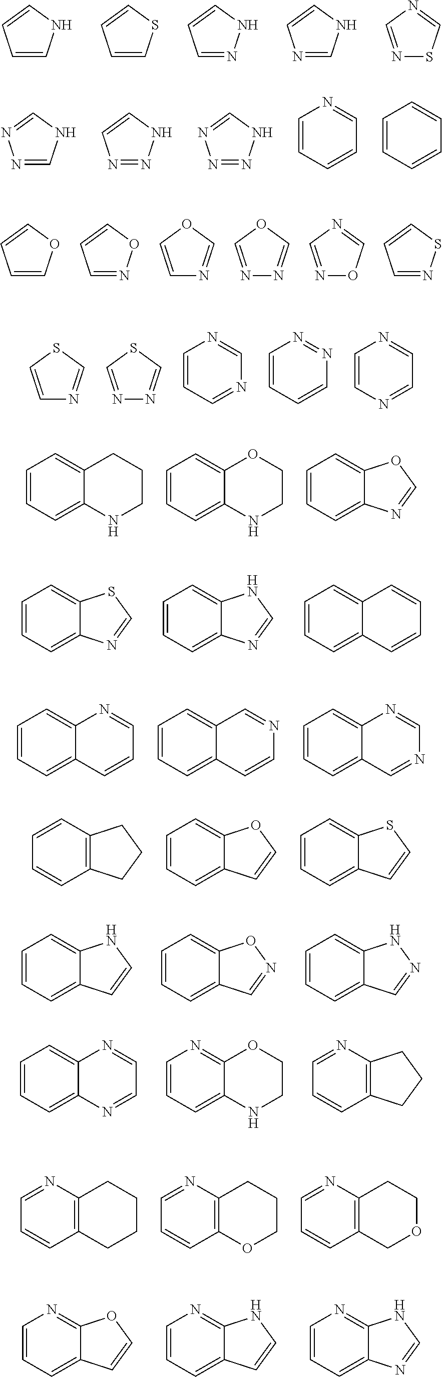

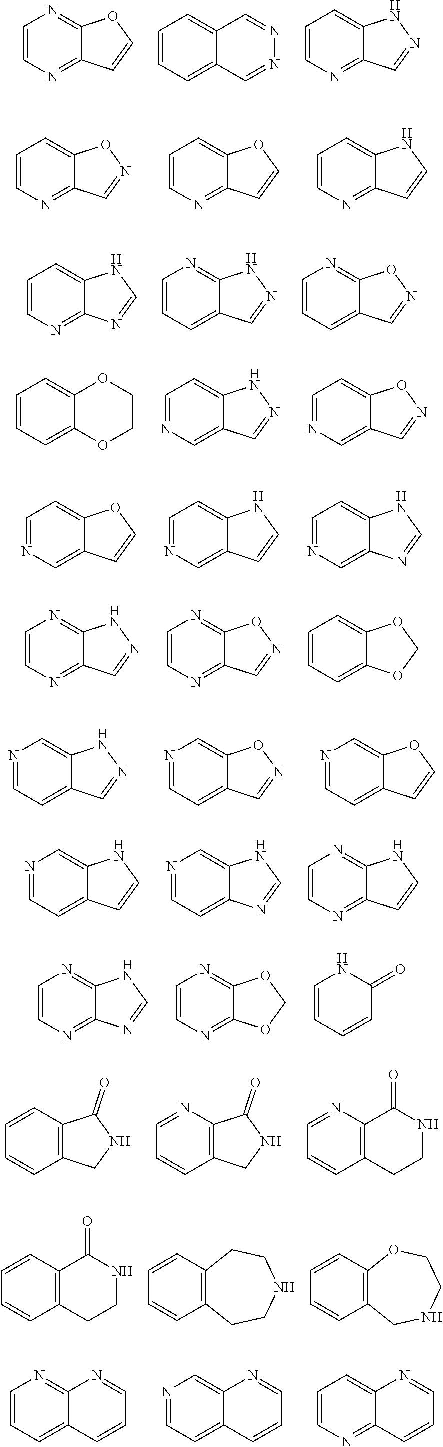

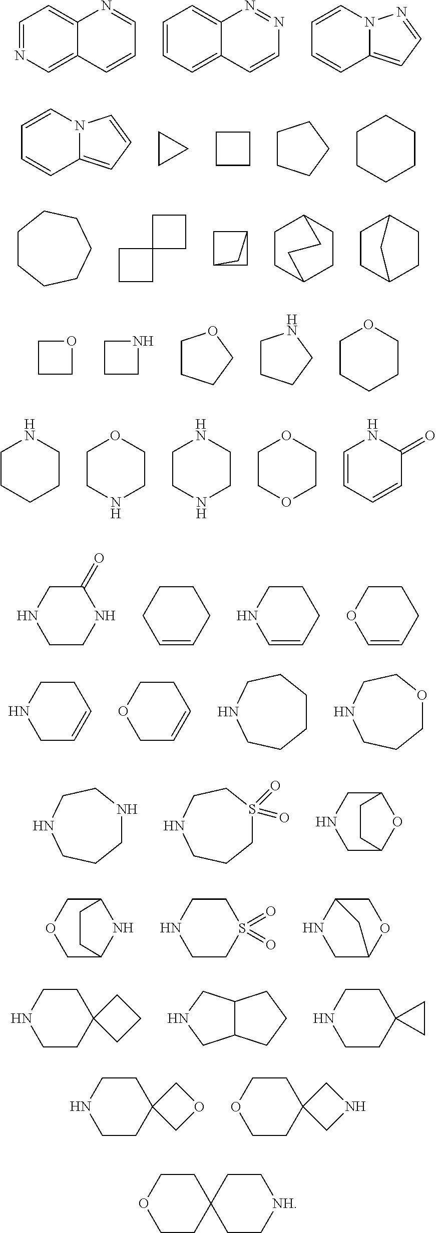

In certain embodiments, the compound of Formula (I) is represented by one of Formulae (XVII-1)˜(XVII-6):

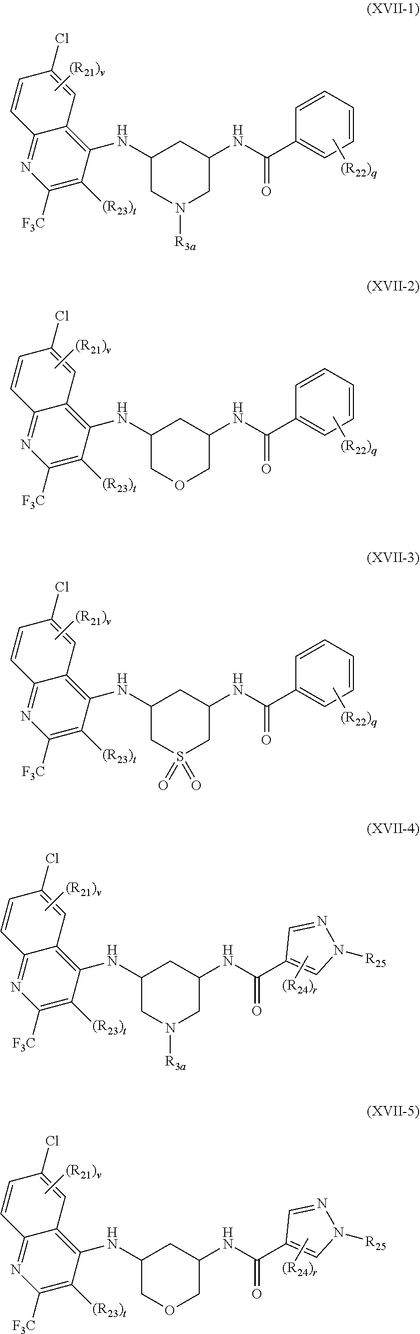

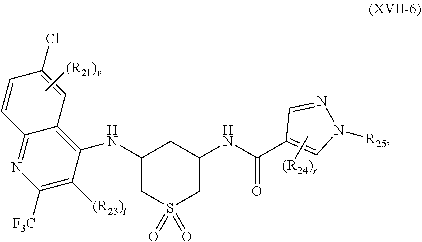

wherein:
each R₂₂ is independently selected from the group consisting of:
   1) halogen;
   2) —CN;
   3) —OH;
   4) —OR¹¹;
   5) —NR¹²R¹³;
   6) Optionally substituted —C₁-C₈ alkyl;
   7) Optionally substituted —C₁-C₈ haloalkyl;
   8) Optionally substituted —C₃-C₈ cycloalkyl;
   9) Optionally substituted 3- to 8-membered heterocycloalkyl;
   10) Optionally substituted aryl; and
   11) Optionally substituted heteroaryl;
each R₂₄ is independently selected from the group consisting of:
   1) halogen;
   2) —NR¹²R¹³;
   3) Optionally substituted —C₁-C₈ alkyl;
   4) Optionally substituted —C₁-C₈ haloalkyl;
   5) Optionally substituted —C₃-C₈ cycloalkyl;
   6) Optionally substituted 3- to 8-membered heterocycloalkyl;
   7) Optionally substituted aryl; and
   8) Optionally substituted heteroaryl;
each R₂₅ is independently selected from the group consisting of:
   1) hydrogen;
   2) Optionally substituted —C₁-C₈ alkyl;
   3) Optionally substituted —C₁-C₈ haloalkyl;
   4) Optionally substituted —C₃-C₈ cycloalkyl;
   5) Optionally substituted 3- to 8-membered heterocycloalkyl;
   6) Optionally substituted aryl; and
   7) Optionally substituted heteroaryl;
and t, v, q, r, R₂₁, and R₂₃ are as previously defined.

In certain embodiments, the compound of Formula (I) is represented by one of Formulae (XVII-1a)˜(XVII-6a):

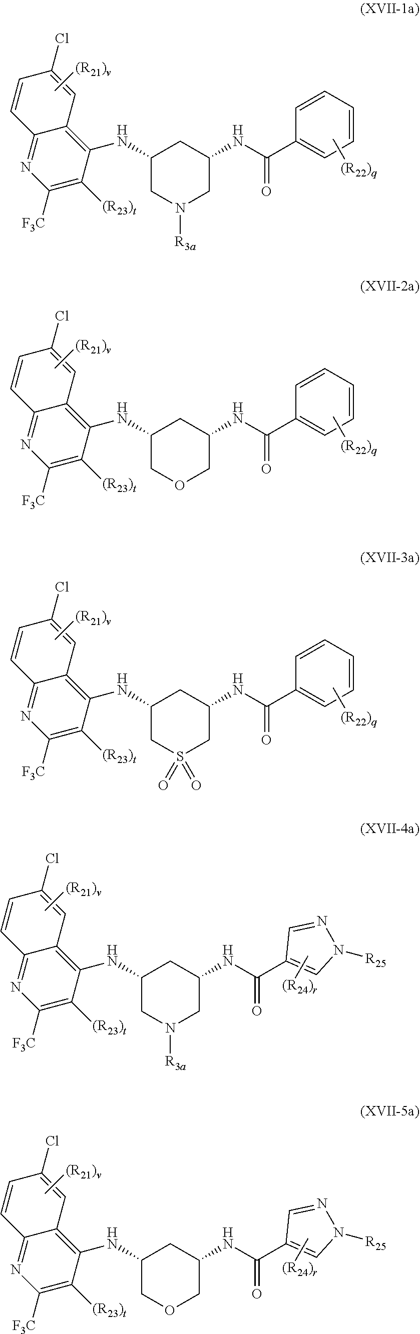

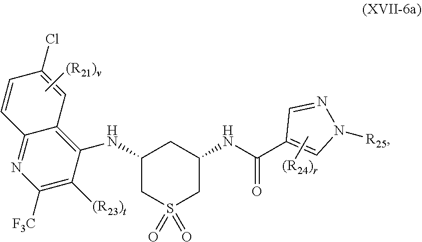

wherein t, v, q, r, R₂₁, R₂₂, R₂₃, R₂₄, and R₂₅ are as previously defined.

Each preferred group stated above can be taken in combination with one, any or all other preferred groups.

It will be appreciated that the description of the present invention herein should be construed in congruity with the laws and principles of chemical bonding. In some instances, it may be necessary to remove a hydrogen atom in order to accommodate a substituent at any given location.

It will be appreciated that the compounds of the present invention may contain one or more asymmetric carbon atoms and may exist in racemic, diastereoisomeric, and optically active forms. It will still be appreciated that certain compounds of the present invention may exist in different tautomeric forms. All tautomers are contemplated to be within the scope of the present invention.

The compounds of the present invention and any other pharmaceutically active agent(s) may be administered together or separately and, when administered separately, administration may occur simultaneously or sequentially, in any order. The amounts of the compounds of the present invention and the other pharmaceutically active agent(s) and the relative timings of administration will be selected in order to achieve the desired combined therapeutic effect. The administration in combination of a compound of the present invention and salts, solvates, or other pharmaceutically acceptable derivatives thereof with other treatment agents may be achieved by concomitant administration in: (1) a unitary pharmaceutical composition including both compounds; or (2) separate pharmaceutical compositions each including one of the compounds.

In certain embodiments of the combination therapy, the additional therapeutic agent is administered at a lower dose and/or dosing frequency as compared to dose and/or dosing frequency of the additional therapeutic agent required to achieve similar results in treating or preventing as PDGFR and/or c-kit kinases inhibitors.

It should be understood that the compounds encompassed by the present invention are those that are suitably stable for use as pharmaceutical agent.

### Definitions

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono- or polycyclic carbocyclic ring system comprising at least one aromatic ring. Preferred aryl groups are C₆-C₁₂-aryl groups, including, but not limited to, phenyl, naphthyl, tetrahydronaphthyl, indanyl, and indenyl. A polycyclic aryl is a polycyclic ring system that comprises at least one aromatic ring. Polycyclic aryls can comprise fused rings, covalently attached rings or a combination thereof.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. In certain embodiments, a heteroaryl group is a 5- to 10-membered heteroaryl, such as a 5- or 6-membered monocyclic heteroaryl or an 8- to 10-membered bicyclic heteroaryl. Heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzoxazolyl, quinoxalinyl. A polycyclic heteroaryl can comprise fused rings, covalently attached rings or a combination thereof. A heteroaryl group can be C-attached or N-attached where possible.

In accordance with the invention, aryl and heteroaryl groups can be substituted or unsubstituted.

The term “bicyclic aryl” or “bicyclic heteroaryl” refers to a ring system consisting of two rings wherein at least one ring is aromatic; and the two rings can be fused or covalently attached.

The term “alkyl” as used herein, refers to saturated, straight- or branched-chain hydrocarbon radicals. “C₁-C₄ alkyl,” “C₁-C₆ alkyl,” “C₁-C₈ alkyl,” “C₁-C₁₂ alkyl,” “C₂-C₄ alkyl,” and “C₃-C₆ alkyl,” refer to alkyl groups containing from 1 to 4, 1 to 6, 1 to 8, 1 to 12, 2 to 4 and 3 to 6 carbon atoms respectively. Examples of alkyl groups include, but are not limited to, methyl, ethyl, propyl, isopropyl, n-butyl, sec-butyl, tert-butyl, n-pentyl, neopentyl, n-hexyl, n-heptyl and n-octyl radicals.

The term “alkenyl” as used herein, refers to straight- or branched-chain hydrocarbon radicals having at least one carbon-carbon double bond. “C₂-C₈ alkenyl,” “C₂-C₁₂ alkenyl,” “C₂-C₄ alkenyl,” “C₃-C₄ alkenyl,” and “C₃-C₆ alkenyl,” refer to alkenyl groups containing from 2 to 8, 2 to 12, 2 to 4, 3 to 4, or 3 to 6 carbon atoms respectively. Alkenyl groups include, but are not limited to, ethenyl, propenyl, butenyl, 2-methyl-2-buten-2-yl, heptenyl, octenyl, and the like.

The term “alkynyl” as used herein, refers to straight- or branched-chain hydrocarbon radicals having at least one carbon-carbon triple bond. “C₂-C₈ alkynyl,” “C₂-C₁₂ alkynyl,” “C₂-C₄ alkynyl,” “C₃-C₄ alkynyl,” and “C₃-C₆ alkynyl,” refer to alkynyl groups containing from 2 to 8, 2 to 12, 2 to 4, 3 to 4, or 3 to 6 carbon atoms respectively. Representative alkynyl groups include, but are not limited to, ethynyl, 2-propynyl, 2-butynyl, heptynyl, octynyl, and the like.

The term “cycloalkyl”, as used herein, refers to a monocyclic or polycyclic saturated carbocyclic ring, such as a bi- or tri-cyclic fused, bridged or spiro system. The ring carbon atoms are optionally oxo-substituted or optionally substituted with an exocyclic olefinic double bond. Preferred cycloalkyl groups include C₃-C₁₂ cycloalkyl, C₃-C₆ cycloalkyl, C₃-C₈ cycloalkyl and C₄-C₇ cycloalkyl. Examples of cycloalkyl include, but are not limited to, cyclopropyl, cyclobutyl, cyclopentyl, cyclohexyl, cyclopentyl, cyclooctyl, 4-methylene-cyclohexyl, bicyclo[2.2.1]heptyl, bicyclo[3.1.0]hexyl, spiro[2.5]octyl, 3-methylenebicyclo[3.2.1]octyl, spiro[4.4]nonanyl, and the like.

The term “cycloalkenyl”, as used herein, refers to monocyclic or polycyclic carbocyclic ring, such as a bi- or tri-cyclic fused, bridged or spiro system having at least one carbon-carbon double bond. The ring carbon atoms are optionally oxo-substituted or optionally substituted with an exocyclic olefinic double bond. Preferred cycloalkenyl groups include C₃-C₁₂ cycloalkenyl, C₄-C₁₂-cycloalkenyl, C₃-C₈ cycloalkenyl, C₄-C₈ cycloalkenyl and C₅-C₇ cycloalkenyl groups. Examples of cycloalkenyl include, but are not limited to, cyclopropenyl, cyclobutenyl, cyclopentenyl, cyclohexenyl, cycloheptenyl, cyclooctenyl, bicyclo[2.2.1]hept-2-enyl, bicyclo[3.1.0]hex-2-enyl, spiro[2.5]oct-4-enyl, spiro[4.4]non-2-enyl, bicyclo[4.2.1]non-3-en-12-yl, and the like.

As used herein, the term “arylalkyl” means a functional group wherein an alkylene chain is attached to an aryl group, e.g., —(CH₂)ₙ-phenyl, where n is 1 to 12, preferably 1 to 6 and more preferably 1 or 2. The term “substituted arylalkyl” means an arylalkyl functional group in which the aryl group is substituted. Similarly, the term “heteroarylalkyl” means a functional group wherein an alkylene chain, is attached to a heteroaryl group, e.g., —(CH₂)ₙ-heteroaryl, where n is 1 to 12, preferably 1 to 6 and more preferably 1 or 2. The term “substituted heteroarylalkyl” means a heteroarylalkyl functional group in which the heteroaryl group is substituted.

As used herein, the term “alkoxy” refers to a radical in which an alkyl group having the designated number of carbon atoms is connected to the rest of the molecule via an oxygen atom. Alkoxy groups include C₁-C₁₂-alkoxy, C₁-C₈-alkoxy, C₁-C₆-alkoxy, C₁-C₄-alkoxy and C₁-C₃-alkoxy groups. Examples of alkoxy groups include, but are not limited to, methoxy, ethoxy, n-propoxy, 2-propoxy (isopropoxy) and the higher homologs and isomers. Preferred alkoxy is C₁-C₃ alkoxy.

An “aliphatic” group is a non-aromatic moiety comprised of any combination of carbon atoms, hydrogen atoms, halogen atoms, oxygen, nitrogen or other atoms, and optionally contains one or more units of unsaturation, e.g., double and/or triple bonds. Examples of aliphatic groups are functional groups, such as alkyl, alkenyl, alkynyl, O, OH, NH, NH₂, C(O), S(O)₂, C(O)O, C(O)NH, OC(O)O, OC(O)NH, OC(O)NH₂, S(O)₂NH, S(O)₂NH₂, NHC(O)NH₂, NHC(O) C(O)NH, NHS(O)₂NH, NHS(O)₂NH₂, C(O)NHS(O)₂, C(O)NHS(O)₂NH or C(O)NHS(O)₂NH₂, and the like, groups comprising one or more functional groups, non-aromatic hydrocarbons (optionally substituted), and groups wherein one or more carbons of a non-aromatic hydrocarbon (optionally substituted) is replaced by a functional group. Carbon atoms of an aliphatic group can be optionally oxo-substituted. An aliphatic group may be straight chained, branched, cyclic, or a combination thereof and preferably contains between about 1 and about 24 carbon atoms, more typically between about 1 and about 12 carbon atoms. In addition to aliphatic hydrocarbon groups, as used herein, aliphatic groups expressly include, for example, alkoxyalkyls, polyalkoxyalkyls, such as polyalkylene glycols, polyamines, and polyimines, for example. Aliphatic groups may be optionally substituted.

The terms “heterocyclic” and “heterocycloalkyl” can be used interchangeably and refer to a non-aromatic ring or a polycyclic ring system, such as a bi- or tri-cyclic fused, bridged or spiro system, where (i) each ring system contains at least one heteroatom independently selected from oxygen, sulfur and nitrogen, (ii) each ring system can be saturated or unsaturated (iii) the nitrogen and sulfur heteroatoms may optionally be oxidized, (iv) the nitrogen heteroatom may optionally be quaternized, (v) any of the above rings may be fused to an aromatic ring, and (vi) the remaining ring atoms are carbon atoms which may be optionally oxo-substituted or optionally substituted with exocyclic olefinic double bond. Representative heterocycloalkyl groups include, but are not limited to, 1,3-dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, 2-azabicyclo[2.2.1]-heptyl, 8-azabicyclo[3.2.1]octyl, 5-azaspiro[2.5]octyl, 2-oxa-7-azaspiro[4.4]nonanyl, 7-oxooxepan-4-yl, and tetrahydrofuryl. Such heterocyclic or heterocycloalkyl groups may be further substituted. A heterocycloalkyl or heterocyclic group can be C-attached or N-attached where possible.

It is understood that any alkyl, alkenyl, alkynyl, alicyclic, cycloalkyl, cycloalkenyl, aryl, heteroaryl, heterocyclic, aliphatic moiety or the like described herein can also be a divalent or multivalent group when used as a linkage to connect two or more groups or substituents, which can be at the same or different atom(s). One skilled in the art can readily determine the valence of any such group from the context in which it occurs.

The term “substituted” refers to substitution by independent replacement of one, two, or three or more of the hydrogen atoms with substituents including, but not limited to, —F, —Cl, —Br, —I, —OH, C₁-C₁₂-alkyl; C₂-C₁₂-alkenyl, C₂-C₁₂-alkynyl, —C₃-C₁₂-cycloalkyl, protected hydroxy, —NO₂, —N₃, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₈-alkenyl, —OCO₂—C₂-C₈-alkynyl, —OCO₂—C₃-C₁₂-cycloalkyl, —OCO₂-aryl, —OCO₂-heteroaryl, —OCO₂-heterocycloalkyl, —CO₂—C₁-C₁₂ alkyl, —CO₂—C₂-C₈ alkenyl, —CO₂—C₂-C₈ alkynyl, —CO₂—C₃-C₁₂-cycloalkyl, —CO₂-aryl, —CO₂-heteroaryl, —CO₂-heterocyloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH-heterocycloalkyl, —NHC(O)H, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₈-alkenyl, —NHCO₂—C₂-C₈-alkynyl, —NHCO₂—C₃-C₁₂-cycloalkyl, —NHCO₂-aryl, —NHCO₂-heteroaryl, —NHCO₂-heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, —NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH₂, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)-heteroaryl, —S(O)-heterocycloalkyl, —SO₂NH₂, —SO₂NH—C₁-C₁₂-alkyl, —SO₂NH—C₂-C₈-alkenyl, —SO₂NH—C₂-C₈-alkynyl, —SO₂—C₁-C₁₂-alkyl, —SO₂—C₂-C₈-alkenyl, —SO₂—C₂-C₈-alkynyl, —SO₂—C₃-C₁₂-cycloalkyl, —SO₂-aryl, —SO₂-heteroaryl, —SO₂-heterocycloalkyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₈-alkenyl, —NHSO₂—C₂-C₈-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, —S-aryl, —S-heteroaryl, —S-heterocycloalkyl, or methylthio-methyl. In certain embodiments, the substituents are independently selected from halo, preferably Cl and F; C₁-C₄-alkyl, preferably methyl and ethyl; halo-C₁-C₄-alkyl, such as fluoromethyl, difluoromethyl, and trifluoromethyl; C₂-C₄-alkenyl; halo-C₂-C₄-alkenyl; C₃-C₆-cycloalkyl, such as cyclopropyl; C₁-C₄-alkoxy, such as methoxy and ethoxy; halo-C₁-C₄-alkoxy, such as fluoromethoxy, difluoromethoxy, and trifluoromethoxy; —CN; —OH; NH₂; C₁-C₄-alkylamino; di(C₁-C₄-alkyl)amino; and NO₂. It is understood that an aryl, heteroaryl, alkyl, alkenyl, alkynyl, cycloalkyl, or heterocycloalkyl in a substituent can be further substituted. In certain embodiments, a substituent in a substituted moiety is additionally optionally substituted with one or more groups, each group being independently selected from C₁-C₄-alkyl; —CF₃, —OCH₃, —OCF₃, —F, —Cl, —Br, —I, —OH, —NO₂, —CN, and —NH₂. Preferably, a substituted alkyl group is substituted with one or more halogen atoms, more preferably one or more fluorine or chlorine atoms.

The term “halo” or halogen” alone or as part of another substituent, as used herein, refers to a fluorine, chlorine, bromine, or iodine atom.

The term “optionally substituted”, as used herein, means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

The term “hydrogen” includes hydrogen and deuterium. In addition, the recitation of an element includes all isotopes of that element so long as the resulting compound is pharmaceutically acceptable. In certain embodiments, the isotopes of an element are present at a particular position according to their natural abundance. In other embodiments, one or more isotopes of an element at a particular position are enriched beyond their natural abundance.

The term “hydroxy activating group,” as used herein, refers to a labile chemical moiety which is known in the art to activate a hydroxyl group so that it will depart during synthetic procedures such as in a substitution or an elimination reaction. Examples of hydroxyl activating group include, but not limited to, mesylate, tosylate, triflate, p-nitrobenzoate, phosphonate and the like.

The term “activated hydroxyl,” as used herein, refers to a hydroxy group activated with a hydroxyl activating group, as defined above, including, but not limited to mesylate, tosylate, triflate, p-nitrobenzoate, phosphonate groups.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in P. G. M. Wuts, Greene's Protective Groups in Organic Synthesis, 5th edition, John Wiley & Sons, Hoboken, NJ (2014). Examples of hydroxyl protecting groups include, but are not limited to, benzyloxycarbonyl, 4-methoxybenzyloxycarbonyl, tert-butoxy-carbonyl, isopropoxycarbonyl, diphenylmethoxycarbonyl, 2,2,2-trichloroethoxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, allyl, benzyl, triphenyl-methyl (trityl), methoxymethyl, methylthiomethyl, benzyloxymethyl, 2-(trimethylsilyl)-ethoxymethyl, methanesulfonyl, trimethylsilyl, triisopropylsilyl, and the like.

The term “protected hydroxy,” as used herein, refers to a hydroxy group protected with a hydroxy protecting group, as defined above, including but not limited to, benzoyl, acetyl, trimethylsilyl, tricthylsilyl, methoxymethyl groups, for example.

The term “hydroxy prodrug group,” as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in P. G. M. Wuts, Greene's Protective Groups in Organic Synthesis, 5th edition, John Wiley & Sons, Hoboken, NJ (2014). Examples of amino protecting groups include, but are not limited to, methoxycarbonyl, t-butoxycarbonyl, 12-fluorenyl-methoxycarbonyl, benzyloxycarbonyl, and the like.

The term “protected amino,” as used herein, refers to an amino group protected with an amino protecting group as defined above.

The term “leaving group” means a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

The term “protic solvent,” as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

Combinations of substituents and variables envisioned by this invention are only those that result in the formation of stable compounds. The term “stable,” as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. As can be appreciated by the skilled artisan, further methods of synthesizing the compounds of the Formula herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2nd Ed. Wiley-VCH (1999); P.G.M. Wuts, Greene's Protective Groups in Organic Synthesis, 5th edition, John Wiley & Sons, Hoboken, NJ (2014); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject,” as used herein, refers to an animal. Preferably, the animal is a mammal. More preferably, the mammal is a human. A subject also refers to, for example, a dog, cat, horse, cow, pig, guinea pig, fish, bird and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or(S)—, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. Tautomers may be in cyclic or acyclic. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

Certain compounds of the present invention may also exist in different stable conformational forms which may be separable. Torsional asymmetry due to restricted rotation about an asymmetric single bond, for example because of steric hindrance or ring strain, may permit separation of different conformers. The present invention includes each conformational isomer of these compounds and mixtures thereof.

As used herein, the term “pharmaceutically acceptable salt,” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 2-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentane-propionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

As used herein, the term “modulating” MRGPRX2 means that the compound interacts with the MRGPRX2 in a manner such that it functions as an inverse agonist to the receptor, and/or as a competitive antagonist to the receptor.

As used herein, the term “MRGPRX2” refers to one or more of the Mas-related G-protein coupled receptors, which are a group of orphan receptors with limited expression in very specialized tissues (e.g., in mast cells and dorsal root ganglia) and barrier tissues. There are eight related receptors in this class expressed in humans, only four of which have readily identifiable orthologs in other species (i.e., MRGPR D, E, F, and G). The other four receptors (MRGPRX1, X2, X3 and X4) have no counterpart, based on homology, in non-human species. MRGPRX2 refers to a member of the MRGPR family that is expressed on mast cells and is capable of mediating IgE independent activation (e.g., mast cell degranulation) in response to ligand binding.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intra-arterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectable formulations.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, car drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to Van Devanter et al., U.S. Pat. No. 5,508,269 to Smith et al., and WO 98/43650 by Montgomery, all of which are incorporated herein by reference).

### Pharmacology and Administration

The compounds of the invention are useful in the treatment of inflammatory disorders such as atopic dermatitis (AD) (e.g. Asian AD, European AD), chronic urticaria, pseudo-allergic reactions triggered by small molecules (anaphylactoid drug reactions), anaphylactic shock, rosacea, asthma, systemic itch such as cholestatic or uremic itch, chronic itch triggered by systemic diseases, drug-adverse reactions, autoimmune disorders, pain-associated conditions and cancer associated conditions. Therefore, administration of a preferred modulator of MRGPRX2 described herein (e.g. a compound of Formula 1) provides a means to ameliorate symptoms of, and/or provide treatment for various inflammatory diseases and disorders.

Mast cells (MCs) are tissue-resident granulocytes of hematopoietic origin and play a pivotal role in the physiological and pathological inflammatory and immune processes including immunoglobulin E (IgE)-dependent anaphylaxis (Helm et al. 1988), IgE-independent pseudo-allergic reactions (McNeil et al. 2015; Yu et al. 2016), immune surveillance (St John et al. 2011), and modulation of immune responses to microorganisms such as bacteria, viruses, and parasites (Abraham and St John 2010). The classical pathway of MCs activation mediates IgE-dependent anaphylaxis by cross-linking the high affinity IgE receptor FcERI. In addition to FcERI, MCs express a large number of G protein-coupled receptors (GPCRs) for diverse ligands, including lipids, chemokines, adenosine, anaphylatoxins C3a and C5a, and peptides (Ahamed et al. 2004; Jiang et al. 2007; McNeil et al. 2015; Okayama et al. 2008; Wojta et al. 2002). Mas-related G protein-coupled receptor —X2 (MRGPRX2) has been known as a novel receptor that activates MCs.

Mas-related G protein-coupled receptors (MRGPRs) are a class of seven-transmembrane GPCRs comprised of approximately 50 members in mice, rats, humans, and monkeys (Bader et al. 2014). MRGPRs are divided into several sub-families: MrgprA, B, C, D, E, F, G, H, and a primate-specific MRGPRX subfamily (Fujisawa et al. 2014; Subramanian et al. 2016; Zylka et al. 2003). The MRGPRs family was discovered by its expression in specific dorsal root and trigeminal ganglia neurons, MCs, tumors, and cardiovascular organs (Bader et al. 2014; Dong et al. 2001; Tatemoto et al. 2006). MRGPRX2 is most abundant in specific dorsal root and trigeminal ganglia neurons and MCs and has a little expression in both the peripheral and central nervous system (Robas et al. 2003). In human MCs, hMRGPRX2 is the primary MRGRPRs, with small amounts of hMRGPRX1 also being expressed, but not hMRGPRX3 or hMRGPRX4 (Subramanian et al. 2011b; Tatemoto et al. 2006). MRGPRX2 is exclusively expressed in MCTC (which express tryptase and chymase) or connective tissue MCs, such as human skin MCs (HSMC), human cultured MCs which are connective tissue type (HCMCCT), cord blood-derived MCs, CD34+ cell-derived MCs, and human LAD2 MCs (McNeil et al. 2015; Tatemoto et al. 2006). Conversely, MCT and immature MC lines lack or express little of the receptor, such as human cultured MCs which are cultured in serum-containing medium (HCMCM), human mast cell line-1 (HMC-1), and bone marrow-derived MCs (BMMC)(Moon et al. 2003; Wu et al. 2015). In addition to humans, MrgprX2 is also expressed in other primates, including chimpanzees, orangutans, gibbons, macaques, and marmosets (Choi and Lahn 2003).

MRGPRX2 is a non-canonical GPCR that is expressed on human MCs and has physiological and pathological effects on MCs. The characteristics of low affinity and low selectivity allow it to interact with a diverse group of ligands, such as antimicrobial peptides (AMPs), neuropeptides (NPs), mast cell degranulating peptides (MCDPs), and FDA-approved drugs (Bader et al. 2014; Subramanian et al. 2016). Recent studies have demonstrated that MRGPRX2 plays a critical role in promoting MCs-mediated host defense and its activation by AMPs, such as defensins and LL-37, enhances microbial clearance, and promotes wound healing in MCs (Chen et al. 2007; Subramanian et al. 2011a, 2013). The MRGPRX2-mediated activation of MCs in close proximity to nerve endings induced by some NPs, such as substance P (SP), increases degranulation and contributes to neuro-genic inflammation, pain, and itch (Barrocas et al. 1999; Hagermark et al. 1978; McNeil et al. 2015; Tatemoto ct al. 2006). In addition, MRGPRX2 ligands such as compound 48/80 (C48/80), SP, and many FDA-approved drugs are known to induce degranulation of MCs via MRGPRX2, releasing storage granules containing a lot of proinflammatory mediators such as histamine, and causing pseudo-allergic diseases (Che et al. 2018; Lansu et al. 2017; Liu et al. 2017; McNeil et al. 2015; Wang et al. 2016; Yaksh et al. 2019). Activation of MRGPRX2 by the ligands contributes to host defense, immunomodulation, inflammatory diseases, and pseudo-allergic drug reactions (McNeil et al. 2015; Solinski et al. 2014; Subramanian et al. 2016). mMrgprB2 and rMrgprB3 are the mouse and rat orthologs of human MRGPRX2. In mice, the expression of mMrgprB2 is restrictedly detected in connective tissue MCs in skin, gut, and trachea (McNeil et al. 2015; Tatemoto et al. 2006). The peritoneum of the mouse is a major source of connective tissue-type MCs. Mouse peritoneal MCs (MPMC) expresses mMrgprB2. hMRGPRX2 and mMrgprB2 have certain unique characteristics as these receptors are expressed in connective tissue MCs and are activated by some ligands such as SP and C48/80 (McNeil et al. 2015). However, hMRGPRX2 and mMrgprB2 show a considerable difference in the ability of ligands to activate or inhibit these receptors. The EC50 values of most ligands for MrgprB2 are significantly higher than those for MRGPRX2 (McNeil et al. 2015). A few compounds even display a selectively inhibiting effect. Neurokinin 1 receptor (NK-1R) antagonist QWF could inhibit SP-induced activation of MrgprB2, and MRGPRX2 as well as MrgprAl in Hela cells. However, NK-1R antagonists L733060 and aprepitant are dual antagonists of NK-1R and mouse MrgprB2 but not human MRGPRX2 (Azimi et al. 2016) because NK-1R does not mediate SP-induced MCs degranulation and because these NK-1R antagonists can interact with MrgprB2 but not with human MRGPRX2 (McNeil et al. 2015; Azimi et al. 2016). The differential effect of NK-1R antagonists on mouse versus human MRGPRs can explain the inconsistencies with respect to their efficacy in mouse models as compared with a range of human conditions (Azimi et al. 2016). The extracellular (EC) domains and transmembrane extracellular regions (TM-EC) of GPCR contribute to ligand binding, whereas intracellular (IC) domains are involved in G protein coupling (Katritch et al. 2012). The differences in the amino acid sequences of hMRGPRX2 and mMrgprB2, which is only ˜53% overall sequence, 34% N-terminal 60 amino acids sequence, and 47% C-terminal 80 amino acids sequence similarity between these receptors, may contribute to differences in the ability of ligands to act on these receptors (Subramanian et al. 2016; Tatemoto et al. 2006). In rats, rMrgprB3 and B8 are expressed in rat peritoneal MCs (RPMC), alongside low levels of rMrgprB1, B2, B6, and B9, but exhibiting no rMrgprA, rMrgprX1, or any other rMrgprB expression (Tatemoto et al. 2006). Rat basophilic leukemia-2H3 (RBL-2H3) cells also do not express MRGPRX2 (Subramanian et al. 2013).

MRGPRX2 is a non-canonical GPCR expressed on human MCs and plays an important role in host defense, immunomodulation, inflammatory diseases, and pseudo-allergic drug reactions. AMPs such as defensins, LL-37, catestatin, and small-molecule nonpeptide host-defense peptides (smHDPs) have the effect of the clearance of microbial pathogens and play a critical role in host defense. These AMPs have direct antimicrobial activities via interacting with the negatively charged phospholipid moieties and disrupting the membrane of microbial pathogens (Hazlett and Wu 2011). In addition to direct antimicrobial activities, AMPs also activate MCs via MRGPRX2 and cause the release of proinflammatory mediators and the generation of cytokines and chemokines that play an important role in host defense, immunomodulation, and wound healing by causing increased vascular permeability and by recruiting or activating other inflammatory cells, such as macrophages, T cells, and basophilic cells (Chen et al. 2007; Subramanian et al. 2011a, 2013). The positive effects of AMPs-induced MCs activation via MRGPRX2 likely outweigh the risks of developing adverse reactions.

In addition to host defense, MRGPRX2 has been identified as a MC specific receptor which is responsible for pseudo-allergic drug reactions (McNeil et al. 2015). MrgprB2, the mouse ortholog of human MRGPRX2, serves as a model to develop potential therapeutic targets for drug-induced pseudo-allergic drug reactions in mice. MRGPRX2 ligands such as C48/80, SP, and many FDA-approved drugs including neuromuscular blocking agents (NMBAs), fluoroquinolone antibiotics, opioid drugs, and some herbal extracts are known to induce degranulation of MCs, releasing storage granules containing large amounts of proinflammatory mediators such as histamine and cause pseudo-allergic diseases (Che et al. 2018; Lansu et al. 2017; Liu et al. 2017; McNeil et al. 2015; Wang et al. 2016; Yaksh et al. 2019). Therefore, MRGPRX2 is also considered to be a therapeutic target for pseudo-allergic diseases. Some non-selective GPCR inhibitors such as pertussis toxin (PTx) and QWF, cytokines, herbal extracts (including saikosaponin A, resveratrol, quercetin, osthole, genistein, shikonin, piperine, and paeoniflorin), small-molecule MRGPRX2 antagonists, and single-stranded DNA (ssDNA) aptamer drugs were effective in inhibiting or blocking MRGPRX2-mediated signaling and displayed the effect of anti-pseudo-allergic reactions (Callahan et al. 2020; Chen et al. 2007; Ogasawara et al. 2019; Suzuki et al. 2020; Wang et al. 2020b). Discovering and identifying inhibitors or antagonists of MRGPRX2 has become a research focus for anti-pseudo-allergic reactions.

The MRGPRX2-mediated MCs activation induced by NPs, such as SP, plays an important role in the modulation of neurogenic inflammation, pain, and itch (Barrocas et al. 1999; Hagermark et al. 1978; McNeil et al. 2015; Tatemoto et al. 2006). MCs are found in close proximity to nerve endings that can release peptides such as SP. Moreover, tryptase released from MCs activates primary afferent neurons resulting in the release of pre-stored SP via proteinase activated receptor-2 (PAR-2)(Kempkes et al. 2014). SP released from neurons and MCs can provide a positive feedback for further MCs activation via MRGPRX2 that increases MCs degranulation and contributes to neurogenic inflammation, pain, and itch (McNeil et al. 2015; Tatemoto et al. 2006). In addition to neurogenic inflammation, MRGPRX2-mediated MCs activation also contributes to the pathogenesis of chronic inflammatory diseases. The expression of MRGPRX2 in chronic urticaria patients' MCs is higher than that in the healthy subjects (Fujisawa et al. 2014). SP-induced agonists of MRGPRs including AMPs, NPs, MCDPs, and FDA-approved drugs induce degranulation of MCs, releasing storage granules containing quantities of proinflammatory mediators such as histamine. Histamine binds to histamine receptors (HR) including H1R, H2R, H3R, and H4R. The activation of H1R which is expressed on endothelial cells and bronchial smooth muscle cells is involved in pseudo-allergic drug reactions (Borriello et al. 2017; Seifert et al. 2013). The activation of H2R and H4R modulates the migration and activation of immune cells (e.g., MCs, basophils, eosinophils, monocytes, dendritic cells, NK, T cells, Treg, and Th2 cells) and plays a major role in pseudo- or allergic reactions and immune-mediated disorders (Borriello et al. 2017). H3R regulates behavior and body temperature at central nervous (Borriello et al. 2017). In addition to histamine release, AMPs such as human β-defensins (hBDs), LL-37, and angiogenic peptide-30/5C (AG-30/5C) induce production of chemokines (such as interleukin 8 (IL-8), monocyte chemoattractant proteins (MCPs) s, and macrophage inflammatory protein (MIPs)) and cytokines (tumor necrosis factor-α (TNFα), prostaglandins (PGs), and cytokines granulocyte-macrophage colony-stimulating factor (GM-CSF))(Chen et al. 2007; Kanazawa et al. 2016; Niyonsaba et al. 2019; Subramanian et al. 2011a). These chemokines and cytokines contribute to host defense, immunomodulation, and wound healing by recruiting or activating inflammatory cells, such as MCs, lymphocytes, macrophages, memory T cells, and basophilic cells (Griffith et al. 2014; Mukai et al. 2018; Ridiandries et al. 2018). MRGPR-mediated MCs activation by NPs leads to the release of proinflammatory cytokines and chemokines and contributes to neurogenic inflammation, pain, and itch (Green et al. 2019). Moreover, the release of tryptase from MCs activates primary afferent neurons via proteinase activated receptor-2 (PAR-2) resulting in the release of pre-stored NPs (Steinhoff et al. 2000). MRGPRX2-mediated MCs activation con-tributes to host defense, immunomodulation, pain, itching, inflammatory disease, and pseudo-allergic drug reactions by degranulation which releases storage granules containing a wide array of proinflammatory mediators and induces the generation of cytokines and chemokines.

Agonists of MRGPRs on MCs are mainly divided into two major categories: peptides and nonpeptides. Peptide agonists of MRGPRs on MCs primarily include AMPs, NPs, MCDPs, peptide hormones, and other endogenous protein fragments. In addition to C48/80, a majority of nonpeptide agonists of MRGPRs on MCs are FDA-approved drugs, including NMBAs, fluoroquinolone antibiotics, opioids, polymyxins, contrast media, herbal extracts, and others. Also, some compounds have been identified as MRGPRX2-selective ligands by compound libraries.

In accordance with the foregoing, the present invention further provides a method for preventing or treating any of the diseases or disorders described above in a subject in need of such treatment, which method comprises administering to said subject a therapeutically effective amount of a compound of Formula (I), or a pharmaceutically acceptable salt thereof. For any of the above uses, the required dosage will vary depending on the mode of administration, the particular condition to be treated and the effect desired. (See, “Administration and Pharmaceutical Compositions,” infra).

An inhibitory amount or dose of the compounds of the present invention may range from about 0.01 mg/Kg to about 500 mg/Kg, alternatively from about 1 to about 50 mg/Kg. Inhibitory amounts or doses will also vary depending on route of administration, as well as the possibility of co-usage with other agents.

According to the methods of treatment of the present invention, conditions are treated or prevented in a patient such as a human or another animal by administering to the patient a therapeutically effective amount of a compound of the invention, in such amounts and for such time as is necessary to achieve the desired result.

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, preferably from about 1 to about 50 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The compounds of the present invention described herein can, for example, be administered by injection, intravenously, intra-arterial, subdermally, intraperitoneally, intramuscularly, or subcutaneously; or orally, buccally, nasally, transmucosally, topically, in an ophthalmic preparation, or by inhalation, with a dosage ranging from about 0.1 to about 500 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically excipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

When the compositions of this invention comprise a combination of a compound of the Formula described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The said “additional therapeutic or prophylactic agents” includes but not limited to, immune therapies (e.g. interferon), therapeutic vaccines, antifibrotic agents, anti-inflammatory agents such as corticosteroids or NSAIDs, bronchodilators such as beta-2 adrenergic agonists and xanthines (e.g. theophylline), mucolytic agents, anti-muscarinics, anti-leukotrienes, inhibitors of cell adhesion (e.g. ICAM antagonists), antioxidants (e.g. N-acetylcysteine), cytokine agonists, cytokine antagonists, lung surfactants and/or antimicrobial and anti-viral agents (e.g. ribavirin and amantidine). The compositions according to the invention may also be used in combination with gene replacement therapy.

### Abbreviations

Abbreviations which may be used in the descriptions of the scheme and the examples that follow are: Ac for acetyl; ACN or MeCN or CH₃CN for acetonitrile; Boc for t-butoxycarbonyl; Boc₂O for di-tert-butyl dicarbonate; Brine for sodium chloride solution in water; BnOH for benzyl alcohol; BOP for Benzotriazole-1-yl-oxy-tris-(dimethylamino)-phosphonium hexafluorophosphate; Burgess reagent for 1-methoxy-N-triethylammoniosulfonyl-methanimidate; Cbz for benzyloxycarbonyl; Cs2CO3 for cesium carbonate; ClCOCOCl for oxalyl chloride; DCM or CH₂Cl₂ for dichloromethane; DDC for N,N′-dicyclohexylcarbodiimide; CH₃ for methyl; DIPEA or (i-Pr)2EtN or Hunig's base for N,N,-diisopropylethyl amine; DMF for N,N-dimethylformamide; DMAP for 4-dimethylaminopyridine; DMS for dimethylsulfide; DMSO for dimethyl sulfoxide; DPPA for diphenylphosphorazide; EDC for 1-ethyl-3-(3-dimethylaminopropyl) carbodiimide; EtOAc for ethyl acetate; EtOH for ethanol; Fe for iron; Fmoc for fluorenylmethyloxycarbonyl; Ghosez's reagent for 1-chloro-N,N,2-trimethyl-1-propenylamine; HATU for O-(7-azabenzotriazol-2-yl)-N,N,N′,N′,-tetramethyluronium Hexafluoro-phosphate; HCl for hydrogen chloride; H₂O for water; IBX for 2-Iodoxybenzoic acid; K₂CO₃ for potassium carbonate; K₂OsO₄·2H₂O for potassium osmate dihydrate; MeOH for methanol; Mesyl-Cl or MsCl for methanesulfonyl chloride; NaBH₄ for sodium borohydride; NaBH₃CN for sodium cyanoborohydride; NaBH(OAc)₃ for sodium triacetoxyborohydride; NaCl for sodium chloride; NaHCO₃ for sodium bicarbonate or sodium hydrogen carbonate; Na₂CO₃ for sodium carbonate; NaIO₄ for sodium periodate; NaN₃ for sodium azide; NaOH for sodium hydroxide; Na₂SO₄ for sodium sulfate; Na₂S₂O₃ for sodium thiosulfate; NH₃ for ammonia; NH₄Cl for ammonium chloride; NH₄OH for ammonium hydroxide; NH₂OH for hydroxyamine; NO₂ for nitro; PMe₃ for trimethylphosphine; O₃ for ozone; PPh₃ for triphenylphosphine PTSA for p-toluenesulfonic acid; Pd/C for palladium on carbon; Pd(OH)₂ for palladium (II) hydroxide; SnCl₂ for tin (II) chloride; SOCl₂ for thionyl chloride; t-BuOH for tert-butyl alcohol; TEA or Et₃N for triethylamine; TFA for trifluoroacetic acid; TFAA for trifluoroacetic anhydride; THF for tetrahydrofuran; Tosyl-Cl or TsCl for p-toluenesulfonyl chloride; TMSN₃ for trimethylsilyl azide; T₃P for propanephosphonic acid anhydride; OTf for triflate; Ph for phenyl; rt for room temperature; TBS for tert-butyl dimethylsilyl; TMS for trimethylsilyl; or TMSOTf for trimethylsilyl trifluoromethanesulfonate; Zn for zinc.

### Synthetic Methods

The compounds and processes of the present invention will be better understood in connection with the following synthetic schemes that illustrate the methods by which the compounds of the invention may be prepared, which are intended as an illustration only and not to limit the scope of the invention. Various changes and modifications to the disclosed embodiments will be apparent to those skilled in the art and such changes and modifications including, without limitation, those relating to the chemical structures, substituents, derivatives, and/or methods of the invention may be made without departing from the spirit of the invention and the scope of the appended claims.

Non-limiting examples of synthetic schemes demonstrating the making of compounds of the invention are illustrated in Schemes 1.

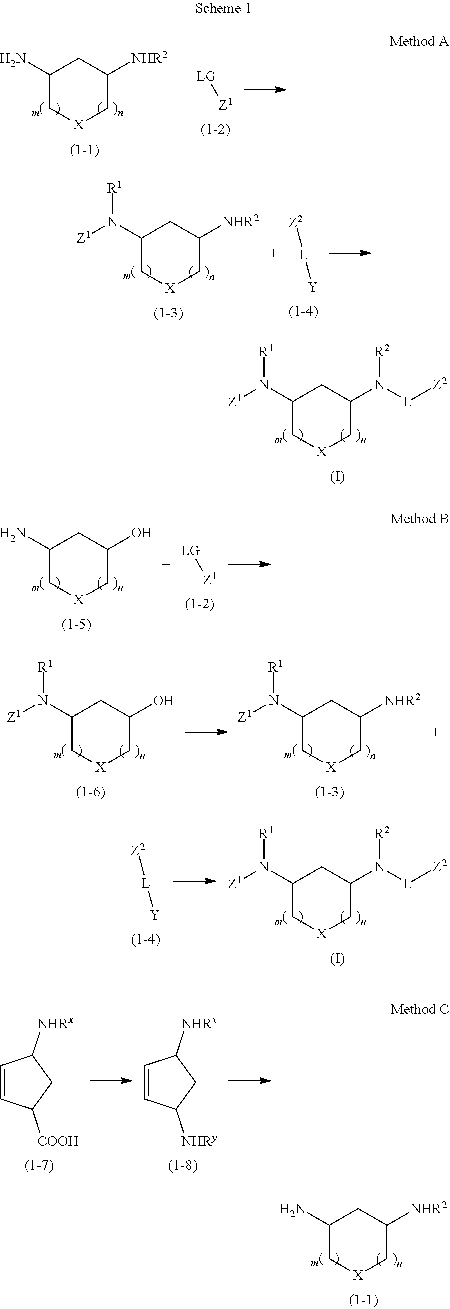

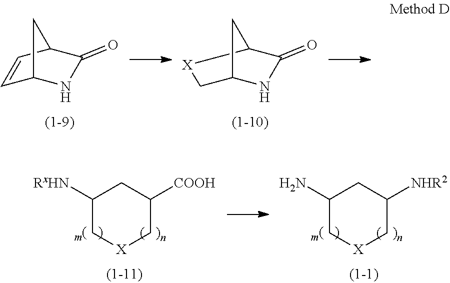

Scheme 1 illustrates a general method to prepare the compound of Formula (I).

In Method A, the amine (1-1), wherein R² and X are as previously defined, reacted with compound (1-2) wherein, Z¹ was previously defined, and LG could be Br, Cl, OTs, etc., under basic conditions (e.g. Hunig's base, Cs₂CO₃, etc.) to provides amine (1-3). The amine (1-3) was condensed with compound (1-4) wherein, Z² and L are previously defined, Y could be leaving groups (e.g. OH, Cl, etc.) under amide coupling conditions (e.g. HATU, EDC, DCC, T₃P, etc.) or other acid activating conditions such as acyl halide (SOCl₂, ClCOCOCl and Ghosez's reagent), or acyl imidazole (carbonyl diimidazole) to provide the compound with Formula (I).

In Method B, the amine alcohol (1-5), wherein X is as previously defined, reacted with compound (1-2) wherein, Z¹ was previously defined, and LG could be Br, Cl, OTs, etc., under basic conditions (e.g. Hunig's base, Cs₂CO₃, etc.) provides amino alcohol (1-6). The amino alcohol (1-6) was converted to amine (1-3) under oxidation (e.g. IBX, Dess-Martin, Parikh-Doering, etc.)/reductive amination (e.g. NaBH₃CN or NaBH(OAc)₃, etc. with NH₄OAc, etc.) or sulfonylation (e.g. TsCl, MsCl, etc.)/azide displacement (e.g. NaN₃, etc.)/azide reduction (e.g. PPh₃, PMe₃, etc.) conditions. The amine (1-3) was condensed with compound (1-4) wherein, Z² and L are previously defined, Y could be leaving groups (e.g. OH, Cl, etc.) under amide coupling conditions (e.g. HATU, EDC, DCC, T₃P, etc.) or other acid activating conditions such as acyl halide (SOCl₂, ClCOCOCl and Ghosez's reagent), or acyl imidazole (carbonyl diimidazole), to provide the compound with Formula (I).

Method C can be used to prepare amine (1-1). R^{x} and R^{y} could be protecting groups on amines, e.g. Boc, Cbz, Fmoc, etc. Acid (1-7) is converted to amine (1-8) under Curtius rearrangement conditions (e.g. DPPA, TMSN₃, etc. then BnOH, allyl alcohol, etc.). Amine (1-9) was converted into amine (1-3), wherein R¹, R² and X are as previously defined, under oxidative cleavage (e.g. O₃ then DMS or NaBH₄, etc. or K₂OsO₄·2H₂O, NMO then NaIO₄), heterocycle formation via reductive amination with amines and NaBH(OAc)₃, Mitsunobu reaction, sulfide displacement, selective deprotection, etc.

Alternatively, Method D can be used to prepare amine (1-1). Bicyclic compound (1-9) was converted into compound (1-10), wherein R¹, R² and X are as previously defined, under oxidative cleavage (e.g. O₃ then DMS or NaBH₄, etc. or KH₂OsO₄, NMO then NaIO₄), heterocycle formation (reductive amination with amines and NaBH(OAc)₃, Mitsunobu reaction, sulfide displacement, etc.). Under Boc₂O/NaOH conditions, compound (1-10) is converted to acid (1-11). Acid (1-10) is converted to amine (1-1) under Curtius rearrangement conditions (e.g. DPPA, TMSN₃, etc. then BnOH, allyl alcohol, etc.).

A route was used to synthesize key intermediate as depicted below in Scheme 2.

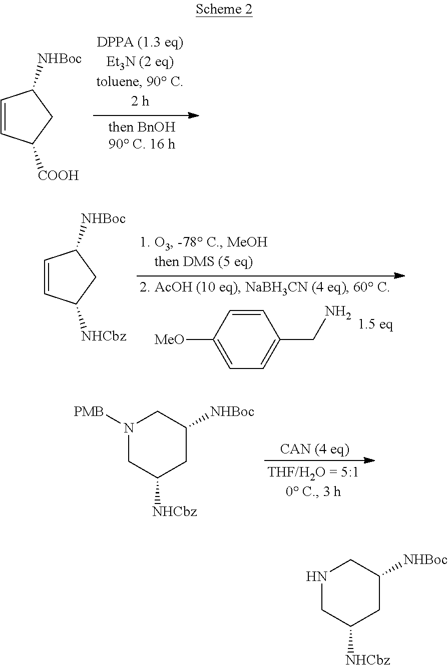

Step 1: (1S,4R)-4-((tert-Butoxycarbonyl)amino)cyclopent-2-ene-1-carboxylic acid (1.828 g, 8.045 mmol), diphenyl phosphorazidate (2.878 g, 10.46 mmol) and triethylamine (1.221 g, 12.07 mmol) were dissolved in toluene (52 mL) and heated at 90° C. for 1 h. Benzyl alcohol (1.740 g, 16.09 mmol) was added and heated at 90° C. for 16 h. The reaction mixture was concentrated and purified by silica gel chromatography to give benzyl tert-butyl (1R,3S)-cyclopent-4-ene-1,3-diyl)dicarbamate (2.24 g, 83.8%). ESI-MS m/z: 333.17 [M+H]⁺.

Step 2: Benzyl tert-butyl (1R,3S)-cyclopent-4-ene-1,3-diyl)dicarbamate 2 (494 mg, 1.49 mmol) was dissolved in MeOH (7.43 mL) and cooled to −78° C. The solution was flushed with ozone until the color changed to light blue. Ozone was removed and the reaction mixture was flushed with oxygen for 2 min until the color changed to colorless. DMS (462 mg, 7.43 mmol) was added and the reaction mixture was allowed to warm to rt for 2 h. (4-methoxyphenyl) methanamine (306 mg, 2.23 mmol) was added and the reaction mixture was allowed to stir at rt for 10 min. acetic acid (892 mg, 14.9 mmol) and sodium cyanoborohydride (374 mg, 5.94 mmol) were added and the reaction mixture was allowed to stir at 60° C. for 16 h. The reaction was quenched by adding saturated NaHCO3 solution. The aqueous layer was extracted with ethyl acetate 3 times. The combined organic layers were dried by magnesium sulfate and concentrated and purified by silica gel chromatography (2% DCM in MeOH) to give benzyl tert-butyl ((3S,5R)-1-(4-methoxybenzyl)piperidine-3,5-diyl)dicarbamate (373 mg, 53.4%). ESI-MS m/z: 470.26 [M+H]⁺.

Step 3: Benzyl tert-butyl (3S,5R)-1-(4-methoxybenzyl)piperidine-3,5-diyl)dicarbamate (182 mg, 388 μmol) was dissolved in THF (3.2 mL) and water (646 μL). The solution was cooled to 0° C. CAN (850 mg, 1.55 mmol) was added slowly. The reaction mixture was allowed to stir at 0° C. for 8 h. LCMS shows some conversion. The reaction mixture was diluted with ethyl acetate and quenched by saturated NaHCO₃ solution. The aqueous layer was extracted with 10% MeOH in DCM 3 times. The combined organic layers were dried with magnesium sulfate and concentrated and purified by silica gel chromatography to give benzyl tert-butyl (3S,5R)-piperidine-3,5-diyl)dicarbamate (85 mg, 63%) ESI-MS m/z: 350.20 [M+H]⁺. and 20% starting material was recovered.

### EXAMPLES

The compounds and processes of the present invention will be better understood in connection with the following examples, which are intended as an illustration only and not limiting the scope of the invention. Starting materials were either available from a commercial vendor or produced by methods well known to those skilled in the art.

### General Conditions:

Mass spectra were run on LC-MS systems using electrospray ionization. These were Agilent 1290 Infinity II systems with an Agilent 6120 Quadrupole detector. Spectra were obtained using a ZORBAX Eclipse XDB-C18 column (4.6×30 mm, 1.8 micron). Spectra were obtained at 298K using a mobile phase of 0.1% formic acid in water (A) and 0.1% formic acid in acetonitrile (B). Spectra were obtained with the following solvent gradient: 5% (B) from 0-1.5 min, 5-95% (B) from 1.5-4.5 min, and 95% (B) from 4.5-6 min. The solvent flowrate was 1.2 mL/min. Compounds were detected at 210 nm and 254 nm wavelengths. [M+H]⁺ refers to mono-isotopic molecular weights.

NMR spectra were run on a Bruker 400 MHz spectrometer. Spectra were measured at 298K and referenced using the solvent peak. Chemical shifts for 1H NMR were reported in parts per million (ppm).

Compounds were purified via reverse-phase high-performance liquid chromatography (RPHPLC) using a Gilson GX-281 automated liquid handling system. Compounds were purified on a Phenomenex Kinetex EVO C18 column (250×21.2 mm, 5 micron), unless otherwise specified. Compounds were purified at 298K using a mobile phase of water (A) and acetonitrile (B) using gradient elution between 0% and 100% (B), unless otherwise specified. The solvent flowrate was 20 mL/min and compounds were detected at 254 nm wavelength.

Alternatively, compounds were purified via normal-phase liquid chromatography (NPLC) using a Teledyne ISCO Combiflash purification system. Compounds were purified on a REDISEP silica gel cartridge. Compounds were purified at 298K and detected at 254 nm wavelength.

### Example 1

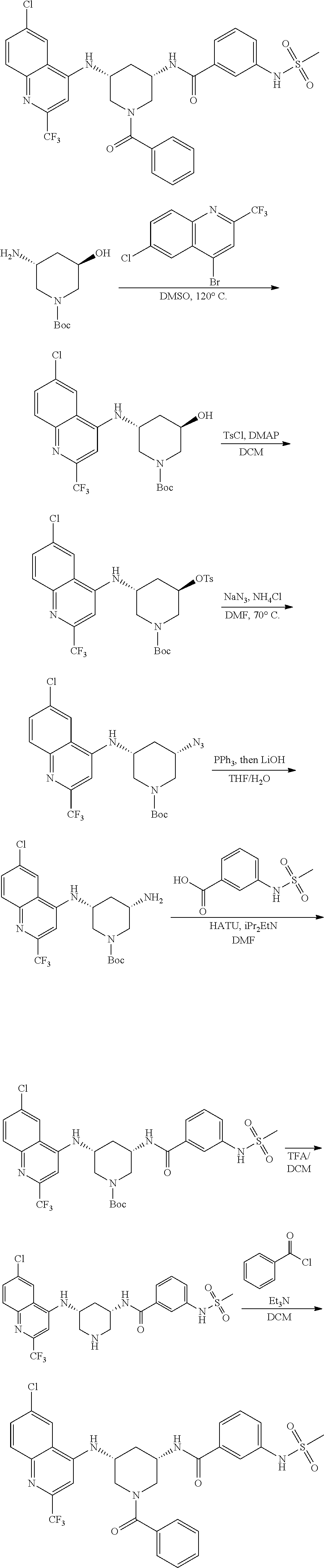

Step 1-1:4-Bromo-6-chloro-2-(trifluoromethyl)quinoline (1.00 g, 3.22 mmol) and tert-butyl (3R,5R)-3-amino-5-hydroxypiperidine-1-carboxylate (697 mg, 3.22 mmol) was dissolved in DMSO (4.95 mL) and Hunig's base (2.24 mL, 12.9 mmol) was added in a vial. The reaction vessel was capped and heated at 120° C. for 2 h. The reaction mixture was diluted with ethyl acetate and washed with brine 3 times. The residue was dried with magnesium sulfate, concentrated in vacuo and purified by silica gel chromatography to give tert-butyl (3R,5R)-3-((6-chloro-2-(trifluoromethyl)quinolin-4-yl)amino)-5-hydroxypiperidine-1-carboxylate (789 mg, 52% yield). ESI-MS m/z: 446.14 [M+H]⁺.

Step 1-2: tert-Butyl (3R,5R)-3-((6-chloro-2-(trifluoromethyl)quinolin-4-yl)amino)-5-hydroxypiperidine-1-carboxylate (100 mg, 224 μmol) was dissolved in DCM (2.24 mL). triethylamine (93.8 μL, 673 μmol) and DMAP (27.4 mg, 224 μmol) were added. The reaction mixture was cooled to 0° C. Tosyl-Cl (64.1 mg, 336 μmol) was added slowly. The reaction mixture was allowed to stir at room temperature for 4 h. The residue was concentrated and purified by silica gel chromatography to give tert-butyl (3R,5R)-3-((6-chloro-2-(trifluoromethyl)quinolin-4-yl)amino)-5-(tosyloxy)piperidine-1-carboxylate (135 mg, 100% yield). ESI-MS m/z: 600.15 [M+H]⁺.

Step 1-3: tert-Butyl (3R,5R)-3-((6-chloro-2-(trifluoromethyl)quinolin-4-yl)amino)-5-(tosyloxy)piperidine-1-carboxylate (340 mg, 567 μmol) was dissolved in DMF (2.83 mL). Ammonium chloride (90.9 mg, 1.70 mmol) and sodium azide (111 mg, 1.70 mmol) were added. The reaction mixture was allowed to stir at 70° C. for 16 h. The reaction mixture was diluted with ethyl acetate and washed with brine 3 times. The residue was dried with magnesium sulfate and concentrated and purified by silica gel chromatography to give tert-butyl (3S,5R)-3-azido-5-((6-chloro-2-(trifluoromethyl)quinolin-4-yl)amino)piperidine-1-carboxylate (212 mg, 79.5% yield). ESI-MS m/z: 471.14 [M+H]⁺.

Step 1-4: tert-Butyl (3S,5R)-3-azido-5-((6-chloro-2-(trifluoromethyl)quinolin-4-yl)amino)piperidine-1-carboxylate (212 mg, 450 μmol) and triphenylphosphine (236 mg, 900 μmol) were dissolved in THF (1.50 mL) and Water (750 μL). The reaction mixture was allowed to stir at room temperature for 16 h. Lithium hydroxide (108 mg, 4.50 mmol) was added and the reaction mixture was allowed to stir at room temperature for 8 h. The reaction mixture was diluted with ethyl acetate and water. The aqueous layer was extracted with ethyl acetate 3 times. The combined organic layers were dried with magnesium sulfate and concentrated and used as crude.

Step 1-5: tert-Butyl (3S,5R)-3-amino-5-((6-chloro-2-(trifluoromethyl)quinolin-4-yl)amino)piperidine-1-carboxylate (250 mg, 562 μmol) in DMF (2.81 mL) from step 1-4 was added 3-(methylsulfonamido)benzoic acid (121 mg, 562 μmol), Hunig's base (0.49 mL, 2.81 mmol), and HATU (321 mg, 843 μmol) at room temperature. The reaction mixture was allowed to stir at room temperature for 3 h. The reaction mixture was diluted with ethyl acetate and washed with brine. The organic layer was dried with magnesium sulfate and concentrated and purified by silica gel chromatography to give tert-butyl (3R,5S)-3-((6-chloro-2-(trifluoromethyl)quinolin-4-yl)amino)-5-(3-(methylsulfonamido)benzamido)piperidine-1-carboxylate (310 mg, 85.9%). ESI-MS m/z: 642.17 [M+H]⁺.

Step 1-6: tert-Butyl (3R,5S)-3-((6-chloro-2-(trifluoromethyl)quinolin-4-yl)amino)-5-(3-(methylsulfonamido)benzamido)piperidine-1-carboxylate (285 mg, 444 μmol) was dissolved in DCM (1.4 mL) and TFA (684 μL, 8.88 mmol) was added. The reaction mixture was allowed to stir at room temperature for 16 h. The reaction mixture was concentrated and used as crude.

Step 1-7: N-((3S,5R)-5-((6-Chloro-2-(trifluoromethyl)quinolin-4-yl)amino)piperidin-3-yl)-3-(methylsulfonamido)benzamide, Trifluoroacetic acid salt (30 mg, 46 μmol) in DCM (0.46 mL) from step 1-6 was added benzoyl chloride (6.4 μL, 55 μmol) and triethylamine (32 μL, 0.23 mmol) at room temperature. The reaction mixture was allowed to stir at room temperature for 5 h. The reaction mixture was concentrated and purified by reverse phase HPLC (20% to 90% ACN in water) to give Example 1 (10 mg, 34% yield) as a white solid. ESI-MS m/z: 646.366 [M+H]⁺.

The following examples were prepared using procedures similar to those described above:

| Example | Structure | ESI-MS |
|---|---|---|
| 2 | 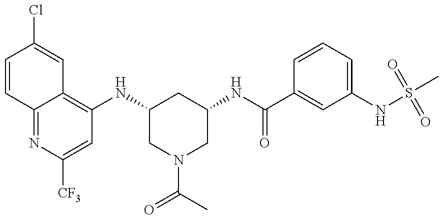 [SMILES: CC(=O)N1C[C@@H](NC(=O)c2cccc(NS(C)(=O)=O)c2)C[C@@H](Nc2cc(C(F)(F)F)nc3ccc(Cl)cc23)C1] | (M + H)⁺: 584.111 |
| 3 | 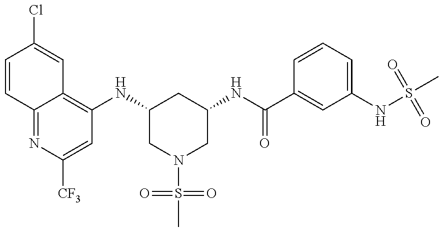 [SMILES: CS(=O)(=O)Nc1cccc(C(=O)N[C@H]2C[C@@H](Nc3cc(C(F)(F)F)nc4ccc(Cl)cc34)CN(S(C)(=O)=O)C2)c1] | (M + H)⁺: 620.109 |
| 4 | 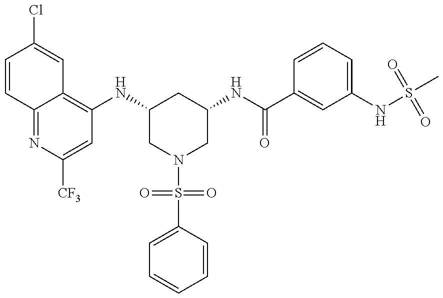 [SMILES: CS(=O)(=O)Nc1cccc(C(=O)N[C@H]2C[C@@H](Nc3cc(C(F)(F)F)nc4ccc(Cl)cc34)CN(S(=O)(=O)c3ccccc3)C2)c1] | (M + H)⁺: 682.224 |
| 5 | 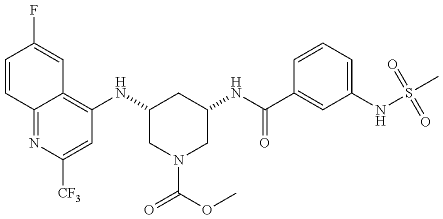 | (M + H)⁺: 600.333 |
| 6 | 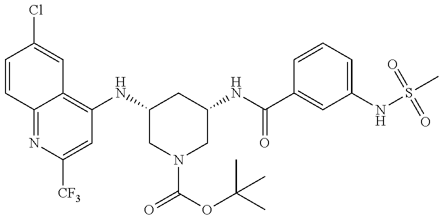 [SMILES: CC(C)(C)OC(=O)N1C[C@@H](NC(=O)c2cccc(NS(C)(=O)=O)c2)C[C@@H](Nc2cc(C(F)(F)F)nc3ccc(Cl)cc23)C1] | (M + H)⁺: 643.006 |
| 7 | 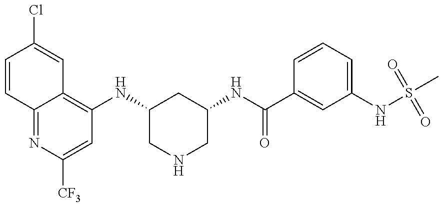 [SMILES: CS(=O)(=O)Nc1cccc(C(=O)N[C@@H]2CNC[C@H](Nc3cc(C(F)(F)F)nc4ccc(Cl)cc34)C2)c1] | (M + H)⁺: 541.963 |
| 8 | 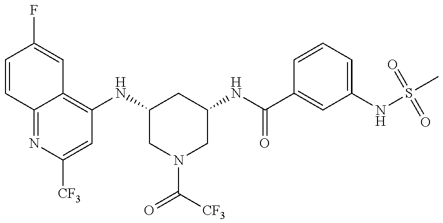 | (M + H)⁺: 638.302 |
| 9 | 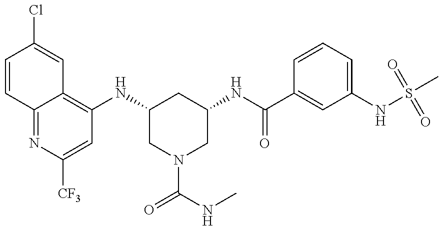 [SMILES: CNC(=O)N1C[C@@H](NC(=O)c2cccc(NS(C)(=O)=O)c2)C[C@@H](Nc2cc(C(F)(F)F)nc3ccc(Cl)cc23)C1] | (M + H)⁺: 599.085 |
| 10 | 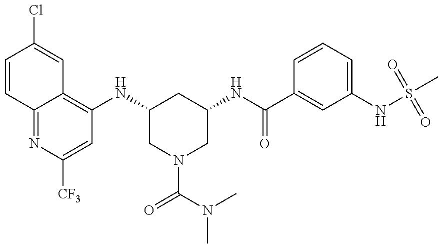 [SMILES: CN(C)C(=O)N1C[C@@H](NC(=O)c2cccc(NS(C)(=O)=O)c2)C[C@@H](Nc2cc(C(F)(F)F)nc3ccc(Cl)cc23)C1] | (M + H)⁺: 613.917 |
| 11 | 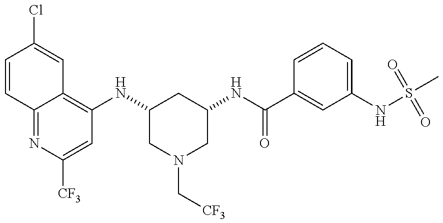 | (M + H)⁺: 624.718 |
| 12 | 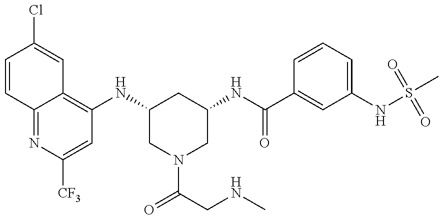 [SMILES: CNCC(=O)N1C[C@@H](NC(=O)c2cccc(NS(C)(=O)=O)c2)C[C@@H](Nc2cc(C(F)(F)F)nc3ccc(Cl)cc23)C1] | (M + H)⁺: 613.014 |
| 13 | 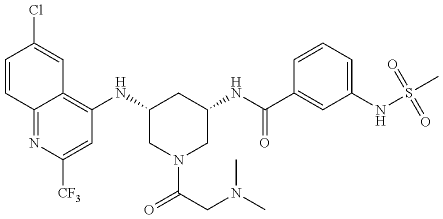 [SMILES: CN(C)CC(=O)N1C[C@@H](NC(=O)c2cccc(NS(C)(=O)=O)c2)C[C@@H](Nc2cc(C(F)(F)F)nc3ccc(Cl)cc23)C1] | (M + H)⁺: 627.165 |
| 14 | 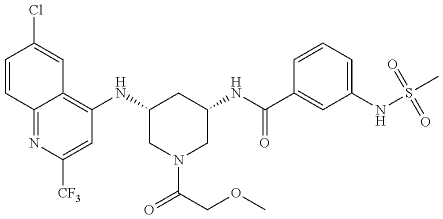 [SMILES: COCC(=O)N1C[C@@H](NC(=O)c2cccc(NS(C)(=O)=O)c2)C[C@@H](Nc2cc(C(F)(F)F)nc3ccc(Cl)cc23)C1] | (M + H)⁺: 614.301 |
| 72 | 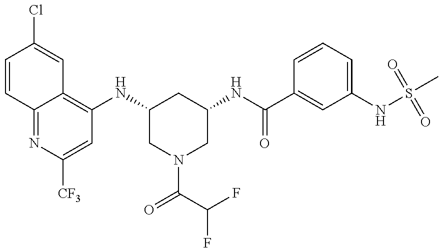 [SMILES: CS(=O)(=O)Nc1cccc(C(=O)N[C@H]2C[C@@H](Nc3cc(C(F)(F)F)nc4ccc(Cl)cc34)CN(C(=O)C(F)F)C2)c1] | (M + H)⁺: 620.11 |
| 73 | 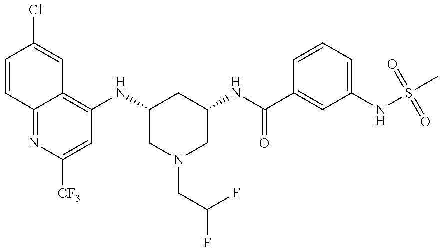 [SMILES: CS(=O)(=O)Nc1cccc(C(=O)N[C@H]2C[C@@H](Nc3cc(C(F)(F)F)nc4ccc(Cl)cc34)CN(CC(F)F)C2)c1] | (M + H)⁺: 606.13 |
| 74 | 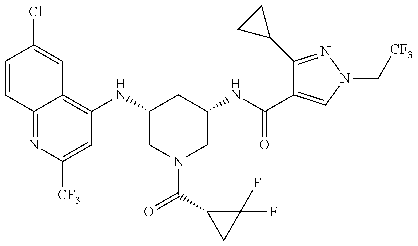 [SMILES: O=C(N[C@H]1C[C@@H](Nc2cc(C(F)(F)F)nc3ccc(Cl)cc23)CN(C(=O)[C@H]2CC2(F)F)C1)c1cn(CC(F)(F)F)nc1C1CC1] | (M + H)⁺: 665.43 |
| 75 | 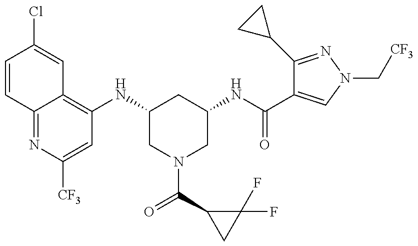 [SMILES: O=C(N[C@H]1C[C@@H](Nc2cc(C(F)(F)F)nc3ccc(Cl)cc23)CN(C(=O)[C@@H]2CC2(F)F)C1)c1cn(CC(F)(F)F)nc1C1CC1] | (M + H)⁺: 665.41 |
| 76 | 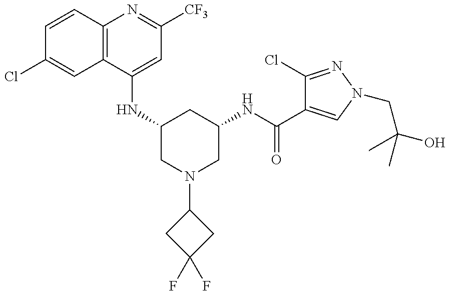 [SMILES: CC(C)(O)Cn1cc(C(=O)N[C@H]2C[C@@H](Nc3cc(C(F)(F)F)nc4ccc(Cl)cc34)CN(C3CC(F)(F)C3)C2)c(Cl)n1] | (M + H)⁺: 635.174 |
| 77 | 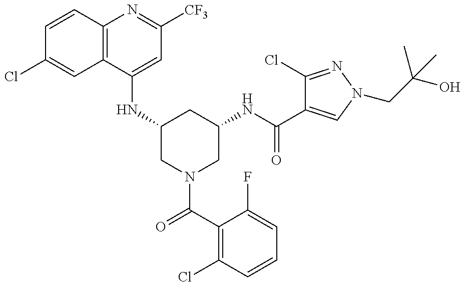 | (M + H)⁺: 617.190 |
| 78 | 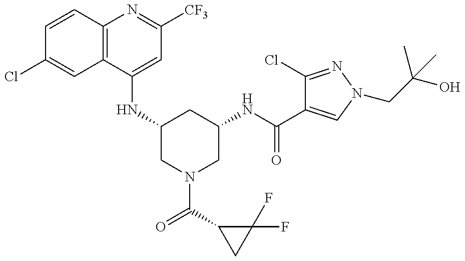 [SMILES: CC(C)(O)Cn1cc(C(=O)N[C@H]2C[C@@H](Nc3cc(C(F)(F)F)nc4ccc(Cl)cc34)CN(C(=O)[C@H]3CC3(F)F)C2)c(Cl)n1] | (M + H)⁺: 649.34 |
| 79 | 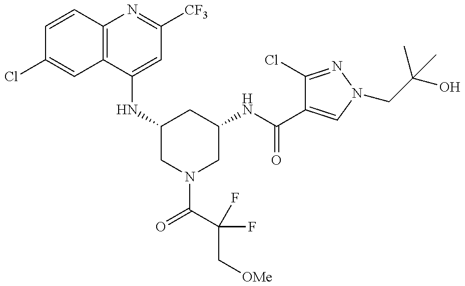 [SMILES: COCC(F)(F)C(=O)N1C[C@@H](NC(=O)c2cn(CC(C)(C)O)nc2Cl)C[C@@H](Nc2cc(C(F)(F)F)nc3ccc(Cl)cc23)C1] | (M + H)⁺: 667.31 |
| 80 | 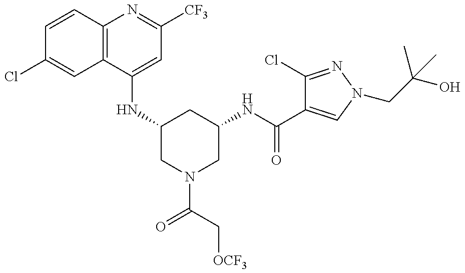 [SMILES: CC(C)(O)Cn1cc(C(=O)N[C@H]2C[C@@H](Nc3cc(C(F)(F)F)nc4ccc(Cl)cc34)CN(C(=O)COC(F)(F)F)C2)c(Cl)n1] | (M + H)⁺: 671.28 |
| 81 | 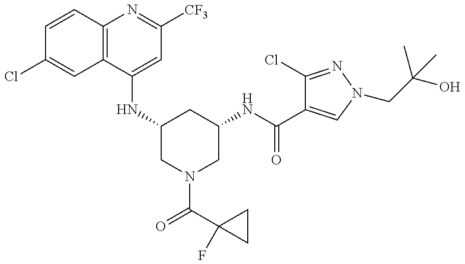 [SMILES: CC(C)(O)Cn1cc(C(=O)N[C@H]2C[C@@H](Nc3cc(C(F)(F)F)nc4ccc(Cl)cc34)CN(C(=O)C3(F)CC3)C2)c(Cl)n1] | (M + H)⁺: 631.32 |
| 82 | 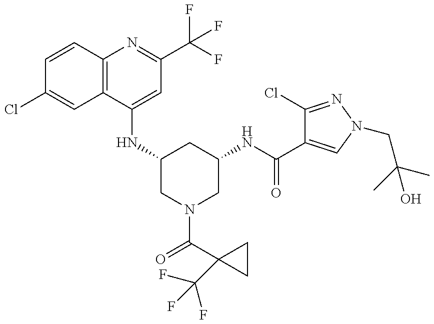 [SMILES: CC(C)(O)Cn1cc(C(=O)N[C@H]2C[C@@H](Nc3cc(C(F)(F)F)nc4ccc(Cl)cc34)CN(C(=O)C3(C(F)(F)F)CC3)C2)c(Cl)n1] | (M + H)⁺: 681.15 |
| 83 | 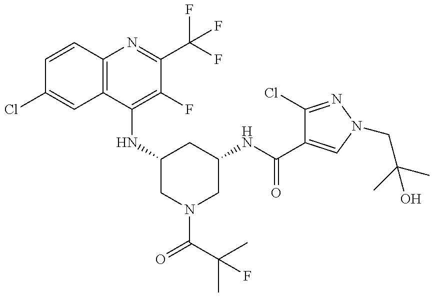 [SMILES: CC(C)(O)Cn1cc(C(=O)N[C@H]2C[C@@H](Nc3c(F)c(C(F)(F)F)nc4ccc(Cl)cc34)CN(C(=O)C(C)(C)F)C2)c(Cl)n1] | (M + H)⁺: 651.16 |
| 84 | 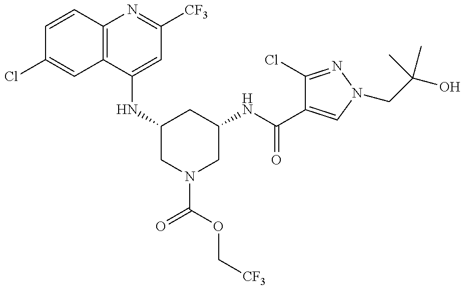 [SMILES: CC(C)(O)Cn1cc(C(=O)N[C@H]2C[C@@H](Nc3cc(C(F)(F)F)nc4ccc(Cl)cc34)CN(C(=O)OCC(F)(F)F)C2)c(Cl)n1] | (M + H)⁺: 671.23 |
| 85 | 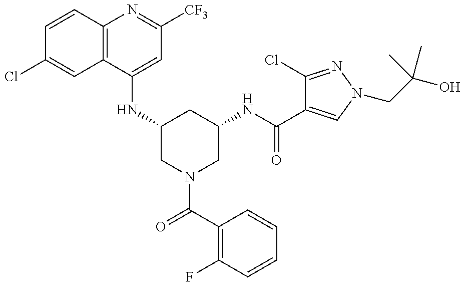 [SMILES: CC(C)(O)Cn1cc(C(=O)N[C@H]2C[C@@H](Nc3cc(C(F)(F)F)nc4ccc(Cl)cc34)CN(C(=O)c3ccccc3F)C2)c(Cl)n1] | (M + H)⁺: 667.28 |
| 86 | 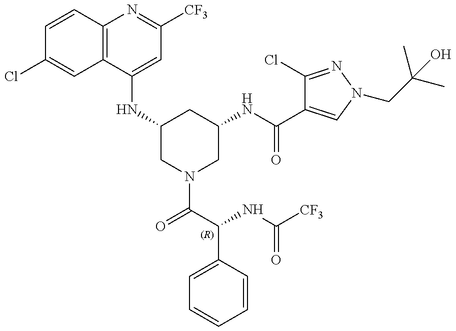 | (M + H)⁺: 774.23 |
| 87 | 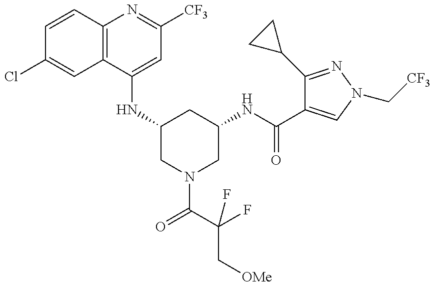 [SMILES: COCC(F)(F)C(=O)N1C[C@@H](NC(=O)c2cn(CC(F)(F)F)nc2C2CC2)C[C@@H](Nc2cc(C(F)(F)F)nc3ccc(Cl)cc23)C1] | (M + H)⁺: 683.52 |
| 88 | 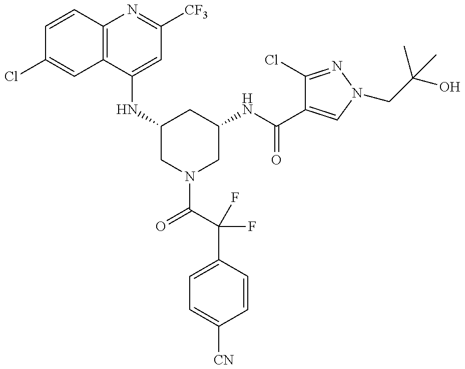 [SMILES: CC(C)(O)Cn1cc(C(=O)N[C@H]2C[C@@H](Nc3cc(C(F)(F)F)nc4ccc(Cl)cc34)CN(C(=O)C(F)(F)c3ccc(C#N)cc3)C2)c(Cl)n1] | (M + H)⁺: 724.27 |
| 89 | 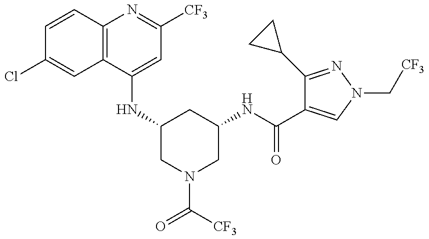 [SMILES: O=C(N[C@H]1C[C@@H](Nc2cc(C(F)(F)F)nc3ccc(Cl)cc23)CN(C(=O)C(F)(F)F)C1)c1cn(CC(F)(F)F)nc1C1CC1] | (M + H)⁺: 657.26 |
| 90 | 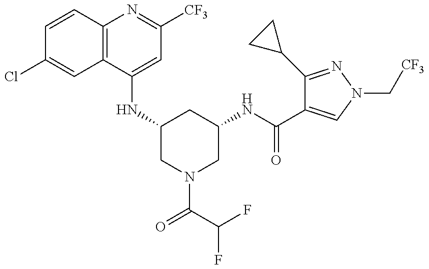 [SMILES: O=C(N[C@H]1C[C@@H](Nc2cc(C(F)(F)F)nc3ccc(Cl)cc23)CN(C(=O)C(F)F)C1)c1cn(CC(F)(F)F)nc1C1CC1] | (M + H)⁺: 639.45 |
| 91 | 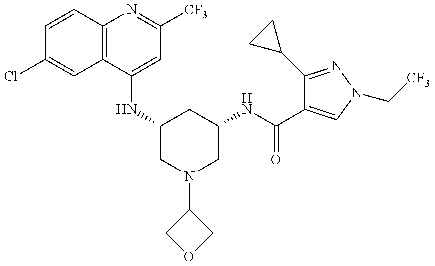 [SMILES: O=C(N[C@H]1C[C@@H](Nc2cc(C(F)(F)F)nc3ccc(Cl)cc23)CN(C2COC2)C1)c1cn(CC(F)(F)F)nc1C1CC1] | (M + H)⁺: 617.19 |
| 92 | 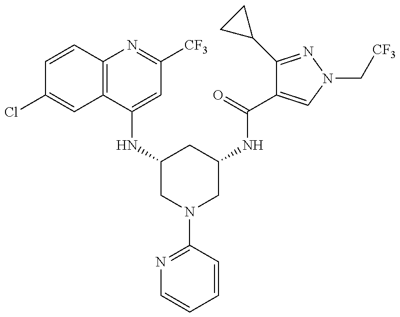 [SMILES: O=C(N[C@H]1C[C@@H](Nc2cc(C(F)(F)F)nc3ccc(Cl)cc23)CN(c2ccccn2)C1)c1cn(CC(F)(F)F)nc1C1CC1] | (M + H)⁺: 637.31 |
| 93 | 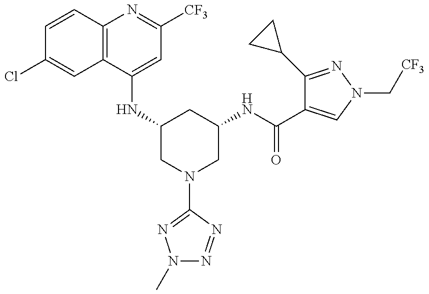 [SMILES: Cn1nnc(N2C[C@@H](NC(=O)c3cn(CC(F)(F)F)nc3C3CC3)C[C@@H](Nc3cc(C(F)(F)F)nc4ccc(Cl)cc34)C2)n1] | (M + H)⁺: 643.19 |

### Example 15

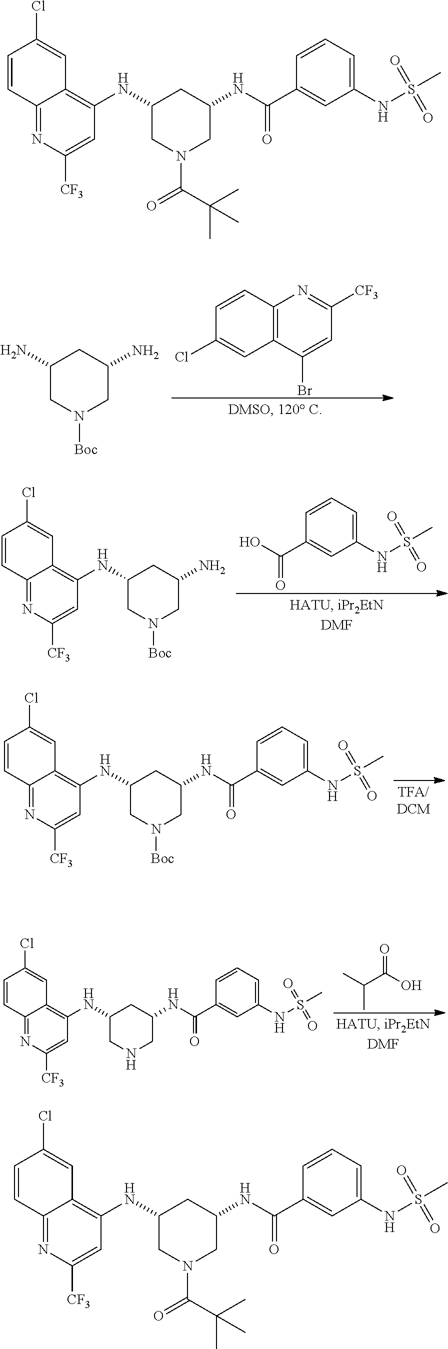

Step 15-1:4-Bromo-6-chloro-2-(trifluoromethyl)quinoline (200 mg, 644 μmol) and tert-butyl cis-3,5-diaminopiperidine-1-carboxylate (277 mg, 1.29 mmol) was dissolved in DMSO (991 μL) and hunig's base (449 μL, 2.58 mmol) was added in a microwave vial. The reaction vessel was capped and heated at 130° C. in the microwave for 30 min. The reaction mixture was used in the next step without further workup.

Step 15-2: tert-Butyl cis-3-amino-5-((6-chloro-2-(trifluoromethyl)quinolin-4-yl)amino)piperidine-1-carboxylate (393 mg, 883 μmol) in DMSO (4.42 mL) from the last step was added 3-(methylsulfonamido)benzoic acid (190 mg, 883 μmol) and HATU (504 mg, 1.33 mmol) at room temperature. The reaction mixture was allowed to stir at room temperature for 5 h. The reaction mixture was diluted with ethyl acetate and washed with brine 3 times. The organic layer was dried with magnesium sulfate and concentrated and purified by silica gel chromatography to give tert-butyl cis-3-((6-chloro-2-(trifluoromethyl)quinolin-4-yl)amino)-5-(3-(methylsulfonamido)benzamido)piperidine-1-carboxylate (567 mg, 100% yield). ESI-MS m/z: 642.17 [M+H]⁺.

Step 15-3: tert-butyl cis-3-((6-chloro-2-(trifluoromethyl)quinolin-4-yl)amino)-5-(3-(methylsulfonamido)benzamido)piperidine-1-carboxylate (632 mg, 984 μmol) was dissolved in DCM (1.5 mL). TFA (1.52 mL, 19.7 mmol) was added. The reaction mixture was allowed to stir at room temperature for 16 h. The reaction mixture was concentrated and used as crude in the next step.

Step 15-4: N-(cis-5-((6-chloro-2-(trifluoromethyl)quinolin-4-yl)amino)piperidin-3-yl)-3-(methylsulfonamido)benzamide, Trifluoroacetic acid salt (100 mg, 152 μmol) in DMF (762 μL) was added pivalic acid (23.4 mg, 229 μmol), Hunig's base (0.49 mL, 2.81 mmol), and HATU (86.9 mg, 229 μmol) at room temperature. The reaction mixture was allowed to stir at room temperature for 5 h. The reaction mixture was diluted with ethyl acetate and washed with brine. The organic layer was dried with magnesium sulfate and concentrated and purified by reverse phase HPLC to give Example 15 (racemic, 20 mg, 21% yield) as a white powder. ESI-MS m/z: 626.350 [M+H]⁺.

The following examples were prepared using procedures similar to those described above:

| Example | Structure | ESI-MS |
|---|---|---|
| 16 | 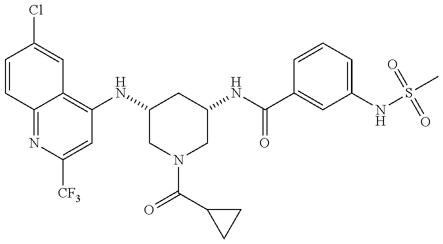 | (M + H)⁺: 610.365 |
| 17 | 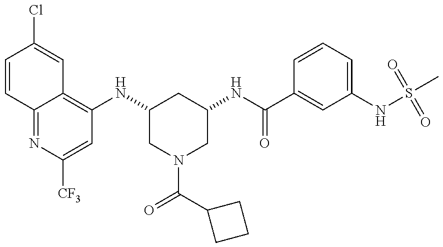 | (M + H)⁺: 624.238 |
| 18 | 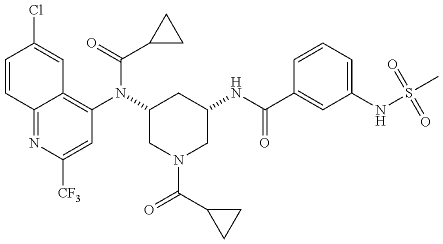 | (M + H)⁺: 678.338 |
| 19 | 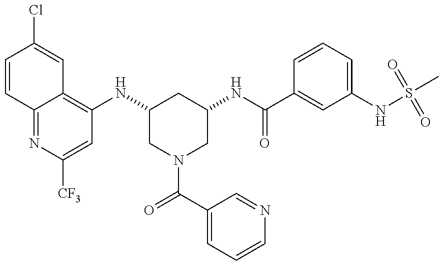 | (M + H)⁺: 647.163 |
| 20 | 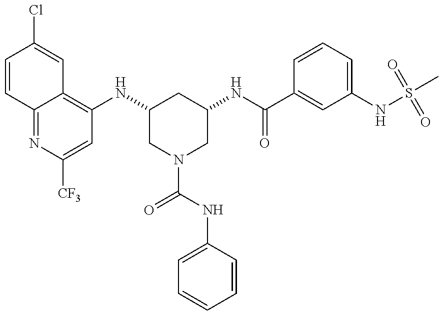 | (M + H)⁺: 661.327 |
| 21 | 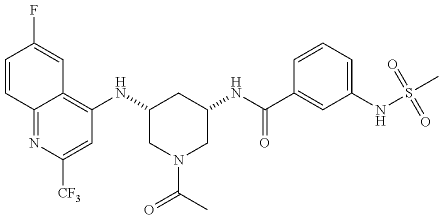 | (M + H)⁺: 568.460 |
| 22 | 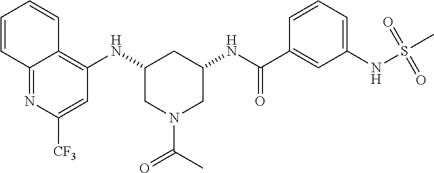 | (M + H)⁺: 550.000 |

### Example 23

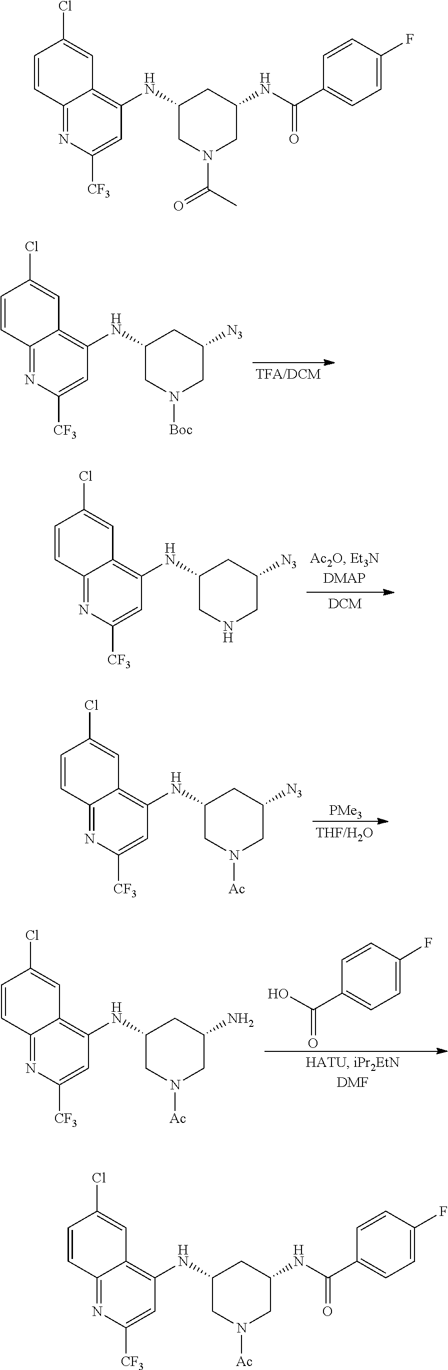

Step 23-1: A mixture of tert-butyl (3S,5R)-3-azido-5-((6-chloro-2-(trifluoromethyl)quinolin-4-yl)amino)piperidine-1-carboxylate (5.21 g, 11.1 mmol) in DCM/TFA (20/10 mL) was stirred at room temperature for 2 h. It was concentrated and used in next step directly.

Step 23-2: To a mixture of N-((3R,5S)-5-azidopiperidin-3-yl)-6-chloro-2-(trifluoromethyl)quinolin-4-amine (4.12 g, 11.1 mmol), DMAP (1.36 g, 11.1 mmol) and Et₃N (7.74 mL, 55.5 mmol) in DCM (55.5 mL) at 0° C. was added acetic anhydride (1.03 mL, 10.9 mmol). The mixture was slowly warmed up to room temperature and allowed to stir for 16 h. The reaction mixture was diluted with ethyl acetate, washed with water and brine. The organic layer was dried over sodium sulfate and concentrated. The residue was purified by silica gel chromatography to give 1-((3S,5R)-3-azido-5-((6-chloro-2-(trifluoromethyl)quinolin-4-yl)amino)piperidin-1-yl) ethan-1-one (4.05 g, 88.4% yield). ESI-MS m/z: 413.10 [M+H]⁺.

Step 23-3: A mixture of 1-((3S,5R)-3-azido-5-((6-chloro-2-(trifluoromethyl)quinolin-4-yl)amino)piperidin-1-yl) ethan-1-one (0.430 g, 1.04 mmol) and trimethylphosphine (2.08 mL, 1.0 molar, 2.08 mmol) in THF (9.47 mL) and water (947 μL). The reaction mixture was allowed to stir at room temperature for 60 h. The reaction mixture was concentrated and used as crude.

Step 23-4: 1-((3S,5R)-3-Amino-5-((6-chloro-2-(trifluoromethyl)quinolin-4-yl)amino)piperidin-1-yl) ethan-1-one (16 mg, 41.4 μmol) in DMF (762 μL) was added 4-fluorobenzoic acid (5.8 mg, 41.4 μmol), Hunig's base (0.49 mL, 2.81 mmol), and HATU (15.7 mg, 41.4 μmol) at room temperature. The reaction mixture was allowed to stir at room temperature for 5 h. The reaction mixture was diluted with ethyl acetate and washed with brine. The organic layer was dried with magnesium sulfate and concentrated and purified by reverse phase HPLC to give Example 23 (9.2 mg, 45% yield) as a white powder. ESI-MS m/z: 509.027 [M+H]⁺.

The following examples were prepared using procedures similar to those described above:

| Example | Structure | ESI-MS |
|---|---|---|
| 24 | 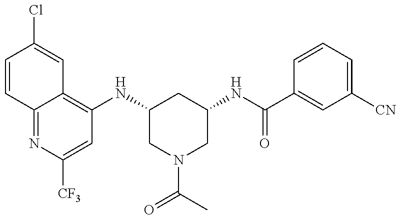 [SMILES: CC(=O)N1C[C@@H](NC(=O)c2cccc(C#N)c2)C[C@@H](Nc2cc(C(F)(F)F)nc3ccc(Cl)cc23)C1] | (M + H)⁺: 516.043 |
| 25 | 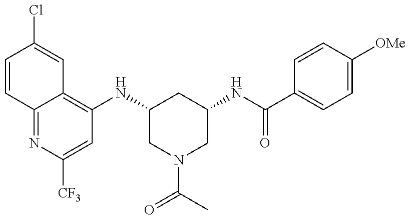 [SMILES: COc1ccc(C(=O)N[C@H]2C[C@@H](Nc3cc(C(F)(F)F)nc4ccc(Cl)cc34)CN(C(C)=O)C2)cc1] | (M + H)⁺: 521.251 |
| 26 | 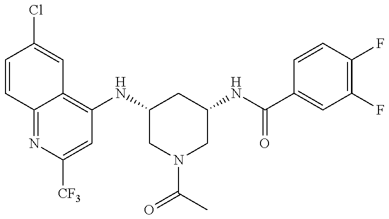 [SMILES: CC(=O)N1C[C@@H](NC(=O)c2ccc(F)c(F)c2)C[C@@H](Nc2cc(C(F)(F)F)nc3ccc(Cl)cc23)C1] | (M + H)⁺: 527.275 |
| 27 | 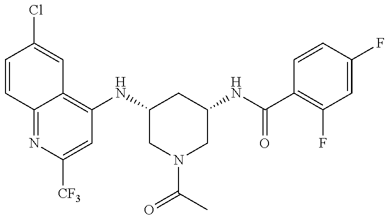 [SMILES: CC(=O)N1C[C@@H](NC(=O)c2ccc(F)cc2F)C[C@@H](Nc2cc(C(F)(F)F)nc3ccc(Cl)cc23)C1] | (M + H)⁺: 527.183 |
| 28 | 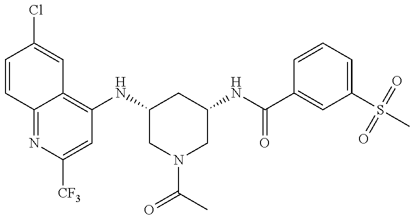 [SMILES: CC(=O)N1C[C@@H](NC(=O)c2cccc(S(C)(=O)=O)c2)C[C@@H](Nc2cc(C(F)(F)F)nc3ccc(Cl)cc23)C1] | (M + H)⁺: 569.038 |
| 29 | 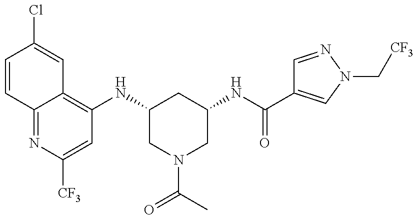 [SMILES: CC(=O)N1C[C@@H](NC(=O)c2cnn(CC(F)(F)F)c2)C[C@@H](Nc2cc(C(F)(F)F)nc3ccc(Cl)cc23)C1] | (M + H)⁺: 563.132 |
| 30 | 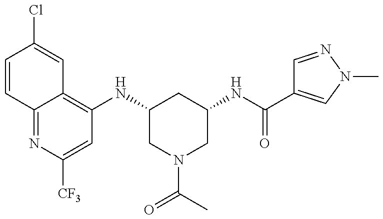 [SMILES: CC(=O)N1C[C@@H](NC(=O)c2cnn(C)c2)C[C@@H](Nc2cc(C(F)(F)F)nc3ccc(Cl)cc23)C1] | (M + H)⁺: 495.370 |
| 31 | 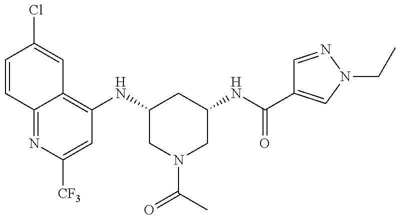 [SMILES: CCn1cc(C(=O)N[C@H]2C[C@@H](Nc3cc(C(F)(F)F)nc4ccc(Cl)cc34)CN(C(C)=O)C2)cn1] | (M + H)⁺: 509.177 |
| 32 | 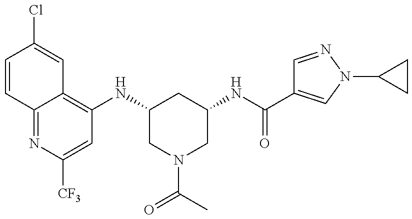 [SMILES: CC(=O)N1C[C@@H](NC(=O)c2cnn(C3CC3)c2)C[C@@H](Nc2cc(C(F)(F)F)nc3ccc(Cl)cc23)C1] | (M + H)⁺: 521.2297 |
| 33 | 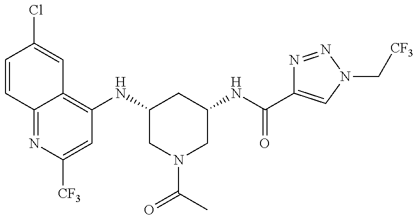 [SMILES: CC(=O)N1C[C@@H](NC(=O)c2cn(CC(F)(F)F)nn2)C[C@@H](Nc2cc(C(F)(F)F)nc3ccc(Cl)cc23)C1] | (M + H)⁺: 564.284 |
| 34 | 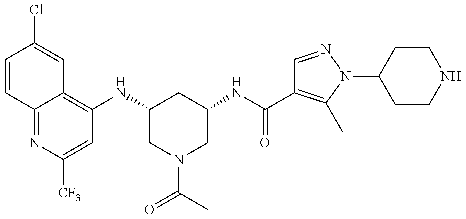 [SMILES: CC(=O)N1C[C@@H](NC(=O)c2cnn(C3CCNCC3)c2C)C[C@@H](Nc2cc(C(F)(F)F)nc3ccc(Cl)cc23)C1] | (M + H)⁺: 578.22 |
| 35 | 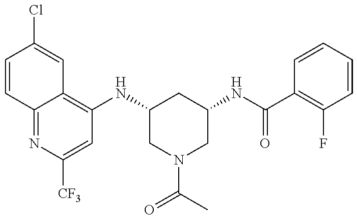 [SMILES: CC(=O)N1C[C@@H](NC(=O)c2ccccc2F)C[C@@H](Nc2cc(C(F)(F)F)nc3ccc(Cl)cc23)C1] | (M + H)⁺: 509.122 |
| 36 | 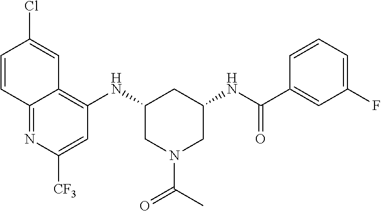 [SMILES: CC(=O)N1C[C@@H](NC(=O)c2cccc(F)c2)C[C@@H](Nc2cc(C(F)(F)F)nc3ccc(Cl)cc23)C1] | (M + H)⁺: 509.192 |
| 37 | 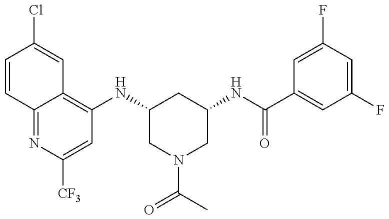 [SMILES: CC(=O)N1C[C@@H](NC(=O)c2cc(F)cc(F)c2)C[C@@H](Nc2cc(C(F)(F)F)nc3ccc(Cl)cc23)C1] | (M + H)⁺: 527.204 |
| 38 | 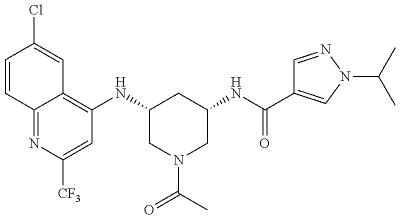 [SMILES: CC(=O)N1C[C@@H](NC(=O)c2cnn(C(C)C)c2)C[C@@H](Nc2cc(C(F)(F)F)nc3ccc(Cl)cc23)C1] | (M + H)⁺: 523.257 |
| 39 | 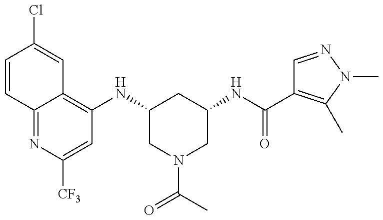 [SMILES: CC(=O)N1C[C@@H](NC(=O)c2cnn(C)c2C)C[C@@H](Nc2cc(C(F)(F)F)nc3ccc(Cl)cc23)C1] | (M + H)⁺: 509.323 |
| 40 | 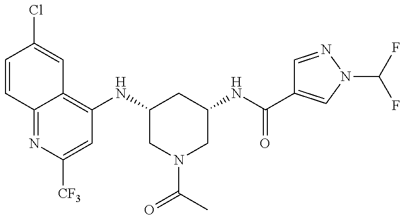 [SMILES: CC(=O)N1C[C@@H](NC(=O)c2cnn(C(F)F)c2)C[C@@H](Nc2cc(C(F)(F)F)nc3ccc(Cl)cc23)C1] | (M + H)⁺: 531.221 |
| 41 | 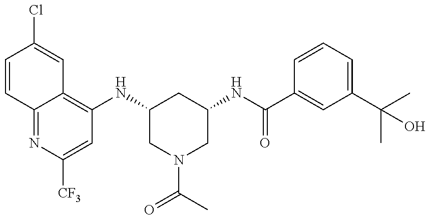 [SMILES: CC(=O)N1C[C@@H](NC(=O)c2cccc(C(C)(C)O)c2)C[C@@H](Nc2cc(C(F)(F)F)nc3ccc(Cl)cc23)C1] | (M + H)⁺: 549.23 |
| 42 | 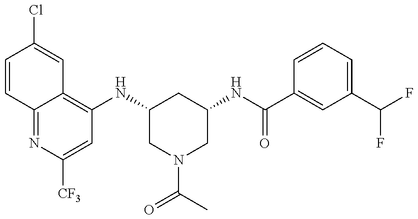 [SMILES: CC(=O)N1C[C@@H](NC(=O)c2cccc(C(F)F)c2)C[C@@H](Nc2cc(C(F)(F)F)nc3ccc(Cl)cc23)C1] | (M + H)⁺: 541.20 |
| 43 | 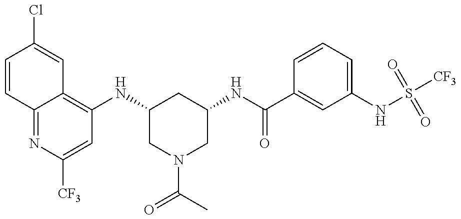 [SMILES: CC(=O)N1C[C@@H](NC(=O)c2cccc(NS(=O)(=O)C(F)(F)F)c2)C[C@@H](Nc2cc(C(F)(F)F)nc3ccc(Cl)cc23)C1] | (M + H)⁺: 638.23 |
| 56 | 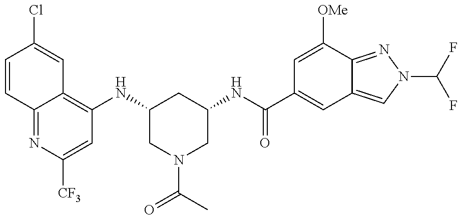 [SMILES: COc1cc(C(=O)N[C@H]2C[C@@H](Nc3cc(C(F)(F)F)nc4ccc(Cl)cc34)CN(C(C)=O)C2)cc2cn(C(F)F)nc12] | (M + H)⁺: 611.277 |
| 57 | 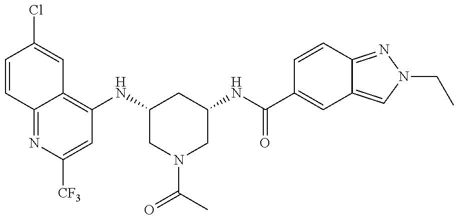 [SMILES: CCn1cc2cc(C(=O)N[C@H]3C[C@@H](Nc4cc(C(F)(F)F)nc5ccc(Cl)cc45)CN(C(C)=O)C3)ccc2n1] | (M + H)⁺: 559.916 |
| 58 | 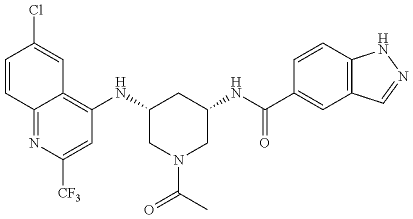 [SMILES: CC(=O)N1C[C@@H](NC(=O)c2ccc3[nH]ncc3c2)C[C@@H](Nc2cc(C(F)(F)F)nc3ccc(Cl)cc23)C1] | (M + H)⁺: 531.211 |
| 59 | 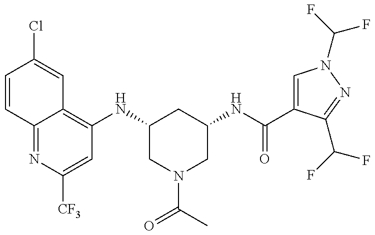 [SMILES: CC(=O)N1C[C@@H](NC(=O)c2cn(C(F)F)nc2C(F)F)C[C@@H](Nc2cc(C(F)(F)F)nc3ccc(Cl)cc23)C1] | (M + H)⁺: 581.852 |
| 60 | 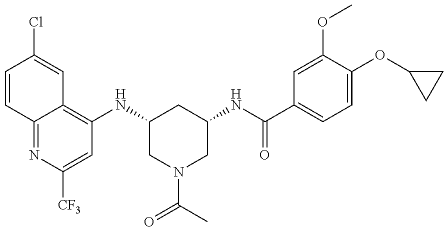 [SMILES: COc1cc(C(=O)N[C@H]2C[C@@H](Nc3cc(C(F)(F)F)nc4ccc(Cl)cc34)CN(C(C)=O)C2)ccc1OC1CC1] | (M + H)⁺: 577.964 |
| 61 | 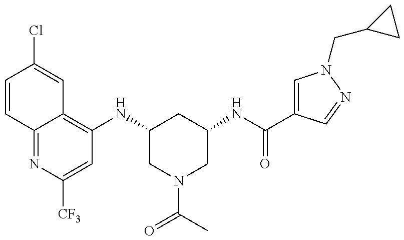 [SMILES: CC(=O)N1C[C@@H](NC(=O)c2cnn(CC3CC3)c2)C[C@@H](Nc2cc(C(F)(F)F)nc3ccc(Cl)cc23)C1] | (M + H)⁺: 535.238 |
| 62 | 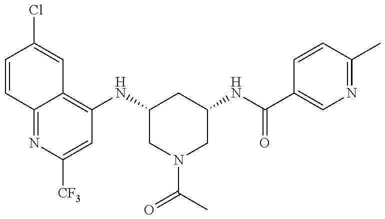 [SMILES: CC(=O)N1C[C@@H](NC(=O)c2ccc(C)nc2)C[C@@H](Nc2cc(C(F)(F)F)nc3ccc(Cl)cc23)C1] | (M + H)⁺: 505.966 |
| 63 | 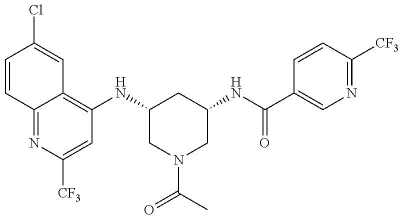 [SMILES: CC(=O)N1C[C@@H](NC(=O)c2ccc(C(F)(F)F)nc2)C[C@@H](Nc2cc(C(F)(F)F)nc3ccc(Cl)cc23)C1] | (M + H)⁺: 560.245 |
| 64 | 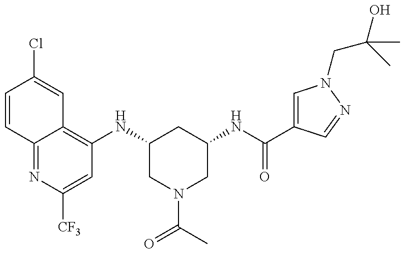 [SMILES: CC(=O)N1C[C@@H](NC(=O)c2cnn(CC(C)(C)O)c2)C[C@@H](Nc2cc(C(F)(F)F)nc3ccc(Cl)cc23)C1] | (M + H)⁺: 553.225 |
| 65 | 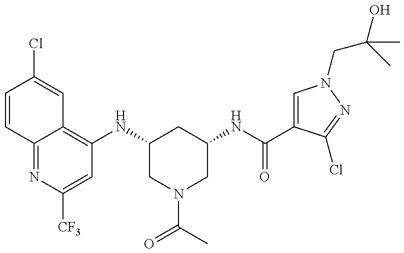 [SMILES: CC(=O)N1C[C@@H](NC(=O)c2cn(CC(C)(C)O)nc2Cl)C[C@@H](Nc2cc(C(F)(F)F)nc3ccc(Cl)cc23)C1] | (M + H)⁺: 587.084 |
| 66 | 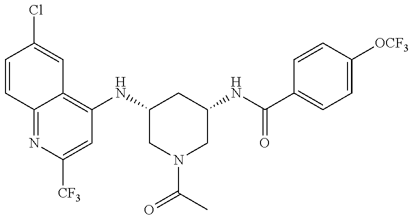 [SMILES: CC(=O)N1C[C@@H](NC(=O)c2ccc(OC(F)(F)F)cc2)C[C@@H](Nc2cc(C(F)(F)F)nc3ccc(Cl)cc23)C1] | (M + H)⁺: 575.142 |
| 67 | 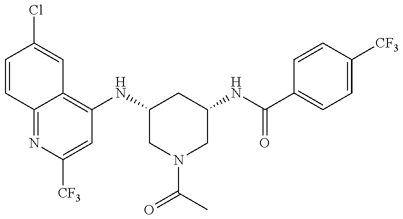 [SMILES: CC(=O)N1C[C@@H](NC(=O)c2ccc(C(F)(F)F)cc2)C[C@@H](Nc2cc(C(F)(F)F)nc3ccc(Cl)cc23)C1] | (M + H)⁺: 559.245 |
| 68 | 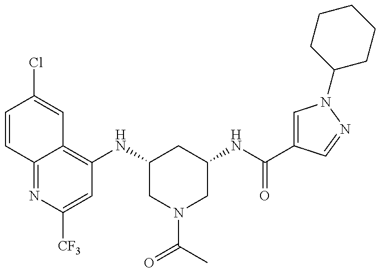 [SMILES: CC(=O)N1C[C@@H](NC(=O)c2cnn(C3CCCCC3)c2)C[C@@H](Nc2cc(C(F)(F)F)nc3ccc(Cl)cc23)C1] | (M + H)⁺: 563.361 |
| 69 | 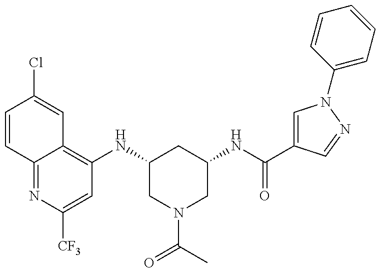 [SMILES: CC(=O)N1C[C@@H](NC(=O)c2cnn(-c3ccccc3)c2)C[C@@H](Nc2cc(C(F)(F)F)nc3ccc(Cl)cc23)C1] | (M + H)⁺: 557.221 |
| 70 | 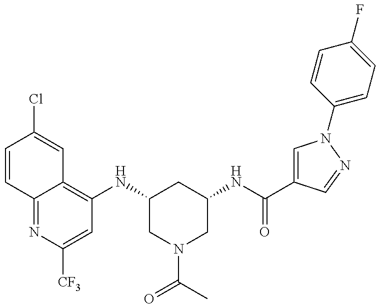 [SMILES: CC(=O)N1C[C@@H](NC(=O)c2cnn(-c3ccc(F)cc3)c2)C[C@@H](Nc2cc(C(F)(F)F)nc3ccc(Cl)cc23)C1] | (M + H)⁺: 575.262 |
| 94 | 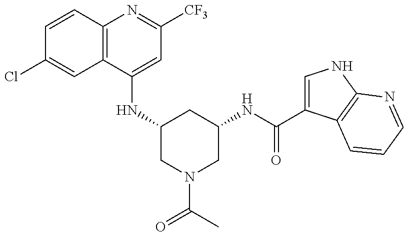 [SMILES: CC(=O)N1C[C@@H](NC(=O)c2c[nH]c3ncccc23)C[C@@H](Nc2cc(C(F)(F)F)nc3ccc(Cl)cc23)C1] | (M + H)⁺: 531.07 |
| 95 | 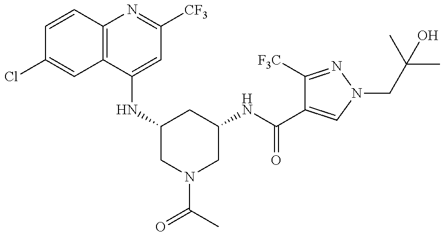 [SMILES: CC(=O)N1C[C@@H](NC(=O)c2cn(CC(C)(C)O)nc2C(F)(F)F)C[C@@H](Nc2cc(C(F)(F)F)nc3ccc(Cl)cc23)C1] | (M + H)⁺: 621.15 |
| 96 | 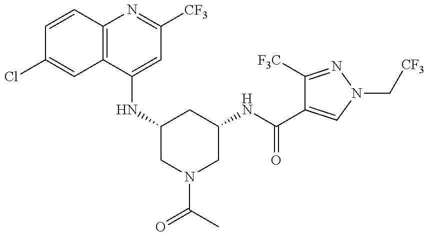 [SMILES: CC(=O)N1C[C@@H](NC(=O)c2cn(CC(F)(F)F)nc2C(F)(F)F)C[C@@H](Nc2cc(C(F)(F)F)nc3ccc(Cl)cc23)C1] | (M + H)⁺: 631.14 |
| 97 | 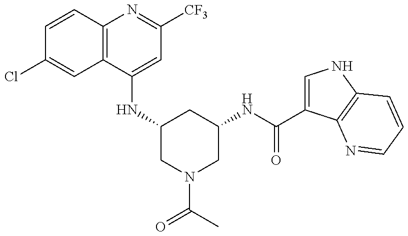 [SMILES: CC(=O)N1C[C@@H](NC(=O)c2c[nH]c3cccnc23)C[C@@H](Nc2cc(C(F)(F)F)nc3ccc(Cl)cc23)C1] | (M + H)⁺: 531.34 |
| 98 | 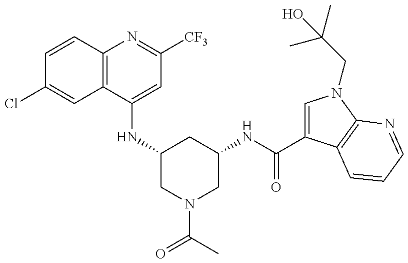 [SMILES: CC(=O)N1C[C@@H](NC(=O)c2cn(CC(C)(C)O)c3ncccc23)C[C@@H](Nc2cc(C(F)(F)F)nc3ccc(Cl)cc23)C1] | (M + H)⁺: 603.20 |
| 99 | 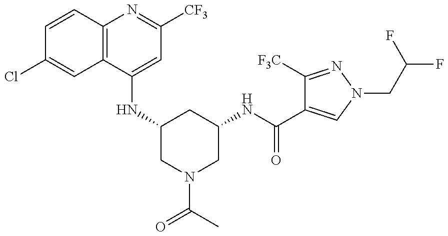 [SMILES: CC(=O)N1C[C@@H](NC(=O)c2cn(CC(F)F)nc2C(F)(F)F)C[C@@H](Nc2cc(C(F)(F)F)nc3ccc(Cl)cc23)C1] | (M + H)⁺: 613.39 |
| 100 | 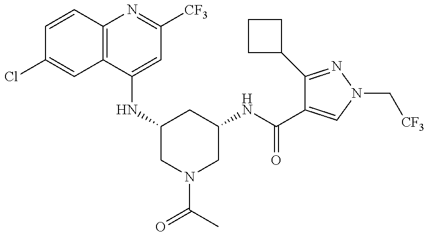 [SMILES: CC(=O)N1C[C@@H](NC(=O)c2cn(CC(F)(F)F)nc2C2CCC2)C[C@@H](Nc2cc(C(F)(F)F)nc3ccc(Cl)cc23)C1] | (M + H)⁺: 617.46 |
| 101 | 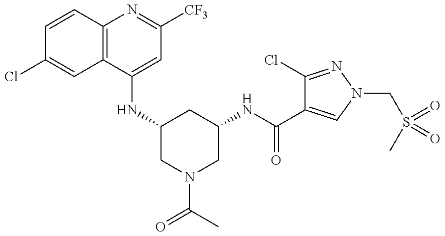 [SMILES: CC(=O)N1C[C@@H](NC(=O)c2cn(CS(C)(=O)=O)nc2Cl)C[C@@H](Nc2cc(C(F)(F)F)nc3ccc(Cl)cc23)C1] | (M + H)⁺: 607.06 |
| 102 | 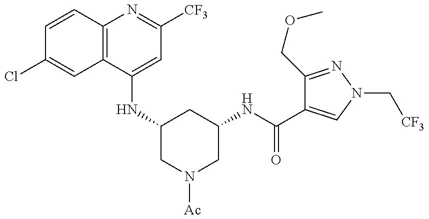 [SMILES: COCc1nn(CC(F)(F)F)cc1C(=O)N[C@H]1C[C@@H](Nc2cc(C(F)(F)F)nc3ccc(Cl)cc23)CN(C(C)=O)C1] | (M + H)⁺: 607.43 |
| 103 | 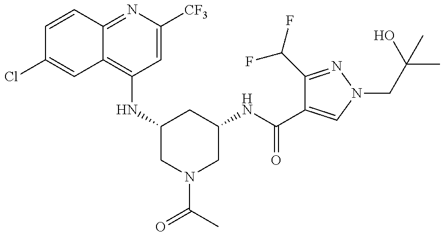 [SMILES: CC(=O)N1C[C@@H](NC(=O)c2cn(CC(C)(C)O)nc2C(F)F)C[C@@H](Nc2cc(C(F)(F)F)nc3ccc(Cl)cc23)C1] | (M + H)⁺: 602.99 |
| 104 | 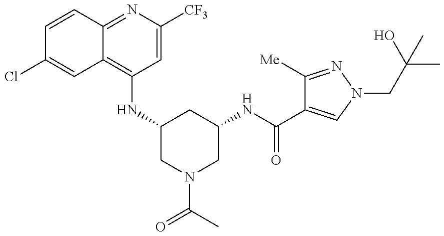 [SMILES: CC(=O)N1C[C@@H](NC(=O)c2cn(CC(C)(C)O)nc2C)C[C@@H](Nc2cc(C(F)(F)F)nc3ccc(Cl)cc23)C1] | (M + H)⁺: 567.98 |
| 105 | 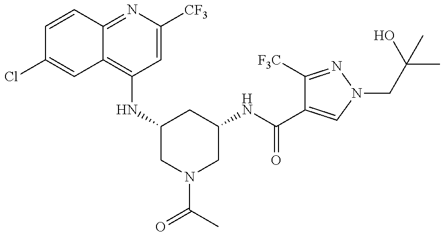 | (M + H)⁺: 631.05 |
| 106 | 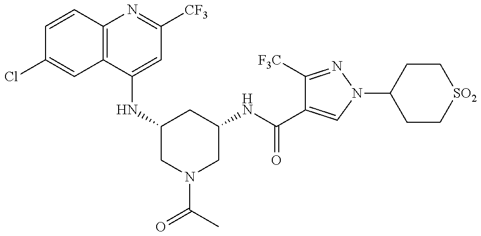 [SMILES: CC(=O)N1C[C@@H](NC(=O)c2cn(C3CCS(=O)(=O)CC3)nc2C(F)(F)F)C[C@@H](Nc2cc(C(F)(F)F)nc3ccc(Cl)cc23)C1] | (M + H)⁺: 681.45 |
| 107 | 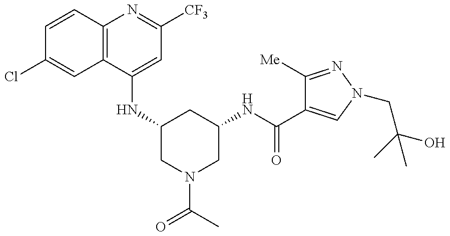 [SMILES: CC(=O)N1C[C@@H](NC(=O)c2cn(CC(C)(C)O)nc2C)C[C@@H](Nc2cc(C(F)(F)F)nc3ccc(Cl)cc23)C1] | (M + H)⁺: 583.54 |
| 108 | 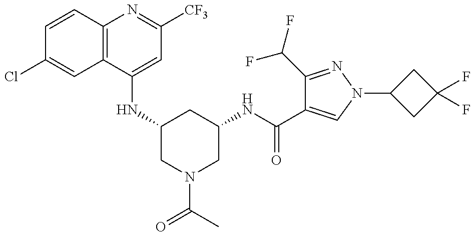 [SMILES: CC(=O)N1C[C@@H](NC(=O)c2cn(C3CC(F)(F)C3)nc2C(F)F)C[C@@H](Nc2cc(C(F)(F)F)nc3ccc(Cl)cc23)C1] | (M + H)⁺: 621.41 |
| 109 | 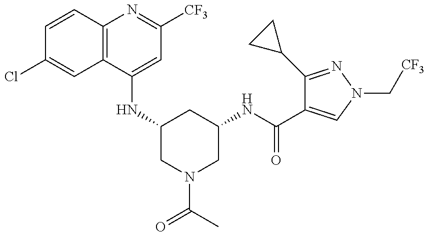 [SMILES: CC(=O)N1C[C@@H](NC(=O)c2cn(CC(F)(F)F)nc2C2CC2)C[C@@H](Nc2cc(C(F)(F)F)nc3ccc(Cl)cc23)C1] | (M + H)⁺: 603.32 |
| 110 | 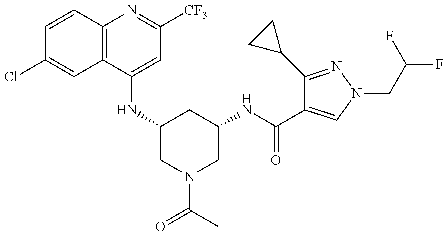 [SMILES: CC(=O)N1C[C@@H](NC(=O)c2cn(CC(F)F)nc2C2CC2)C[C@@H](Nc2cc(C(F)(F)F)nc3ccc(Cl)cc23)C1] | (M + H)⁺: 585.18 |
| 111 | 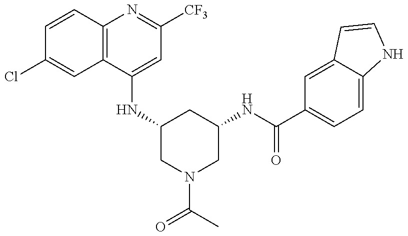 [SMILES: CC(=O)N1C[C@@H](NC(=O)c2ccc3[nH]ccc3c2)C[C@@H](Nc2cc(C(F)(F)F)nc3ccc(Cl)cc23)C1] | (M + H)⁺: 530.25 |
| 112 | 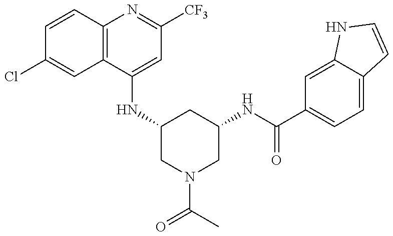 [SMILES: CC(=O)N1C[C@@H](NC(=O)c2ccc3cc[nH]c3c2)C[C@@H](Nc2cc(C(F)(F)F)nc3ccc(Cl)cc23)C1] | (M + H)⁺:530.20 |
| 113 | 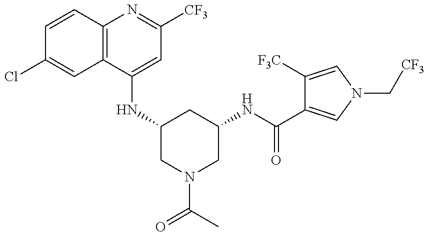 [SMILES: CC(=O)N1C[C@@H](NC(=O)c2cn(CC(F)(F)F)cc2C(F)(F)F)C[C@@H](Nc2cc(C(F)(F)F)nc3ccc(Cl)cc23)C1] | (M + H)⁺: 630.11 |
| 114 | 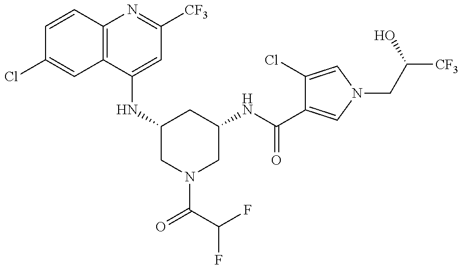 [SMILES: O=C(N[C@H]1C[C@@H](Nc2cc(C(F)(F)F)nc3ccc(Cl)cc23)CN(C(=O)C(F)F)C1)c1cn(C[C@H](O)C(F)(F)F)cc1Cl] | (M + H)⁺: 663.22 |
| 115 | 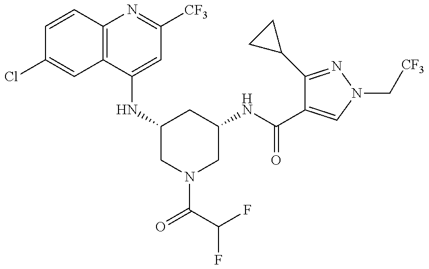 [SMILES: O=C(N[C@H]1C[C@@H](Nc2cc(C(F)(F)F)nc3ccc(Cl)cc23)CN(C(=O)C(F)F)C1)c1cn(CC(F)(F)F)nc1C1CC1] | (M + H)⁺: 639.45 |
| 116 | 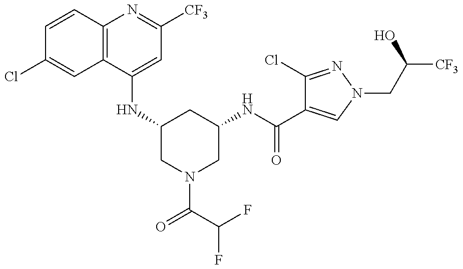 [SMILES: O=C(N[C@H]1C[C@@H](Nc2cc(C(F)(F)F)nc3ccc(Cl)cc23)CN(C(=O)C(F)F)C1)c1cn(C[C@@H](O)C(F)(F)F)nc1Cl] | (M + H)⁺: 663.17 |
| 117 | 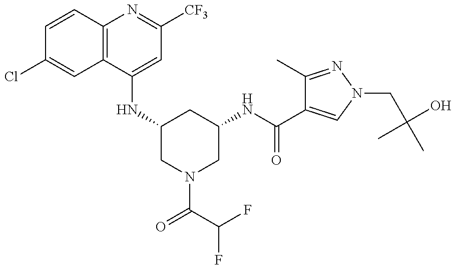 [SMILES: Cc1nn(CC(C)(C)O)cc1C(=O)N[C@H]1C[C@@H](Nc2cc(C(F)(F)F)nc3ccc(Cl)cc23)CN(C(=O)C(F)F)C1] | (M + H)⁺: 603.26 |
| 118 | 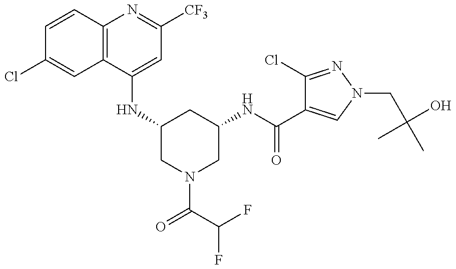 [SMILES: CC(C)(O)Cn1cc(C(=O)N[C@H]2C[C@@H](Nc3cc(C(F)(F)F)nc4ccc(Cl)cc34)CN(C(=O)C(F)F)C2)c(Cl)n1] | (M + H)⁺: 623.23 |
| 119 | 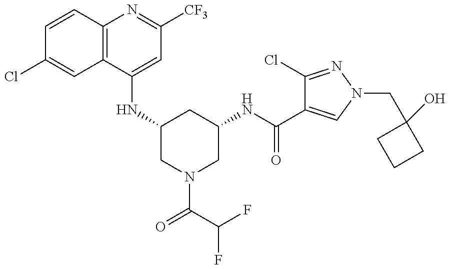 [SMILES: O=C(N[C@H]1C[C@@H](Nc2cc(C(F)(F)F)nc3ccc(Cl)cc23)CN(C(=O)C(F)F)C1)c1cn(CC2(O)CCC2)nc1Cl] | (M + H)⁺: 635.23 |
| 120 | 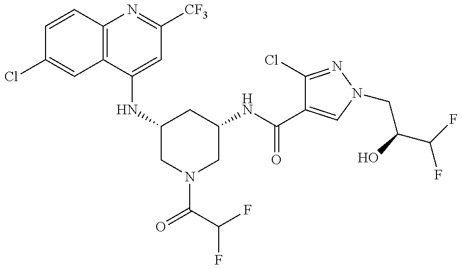 [SMILES: O=C(N[C@H]1C[C@@H](Nc2cc(C(F)(F)F)nc3ccc(Cl)cc23)CN(C(=O)C(F)F)C1)c1cn(C[C@H](O)C(F)F)nc1Cl] | (M + H)⁺: 645.17 |
| 121 | 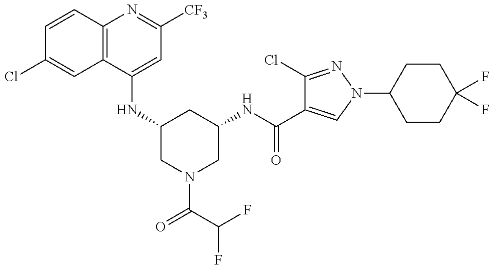 [SMILES: O=C(N[C@H]1C[C@@H](Nc2cc(C(F)(F)F)nc3ccc(Cl)cc23)CN(C(=O)C(F)F)C1)c1cn(C2CCC(F)(F)CC2)nc1Cl] | (M + H)⁺: 669.23 |
| 122 | 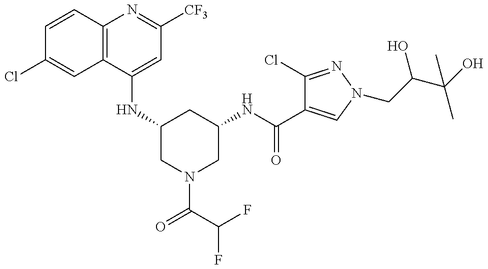 [SMILES: CC(C)(O)C(O)Cn1cc(C(=O)N[C@H]2C[C@@H](Nc3cc(C(F)(F)F)nc4ccc(Cl)cc34)CN(C(=O)C(F)F)C2)c(Cl)n1] | (M + H)⁺: 653.23 |
| 123 | 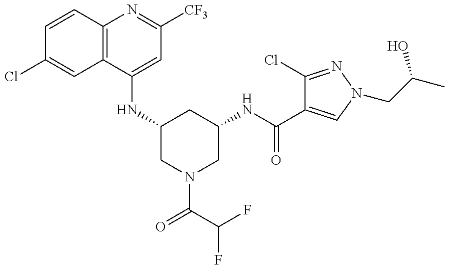 [SMILES: C[C@@H](O)Cn1cc(C(=O)N[C@H]2C[C@@H](Nc3cc(C(F)(F)F)nc4ccc(Cl)cc34)CN(C(=O)C(F)F)C2)c(Cl)n1] | (M + H)⁺: 609.11 |
| 124 | 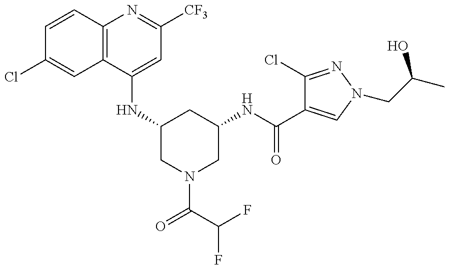 [SMILES: C[C@H](O)Cn1cc(C(=O)N[C@H]2C[C@@H](Nc3cc(C(F)(F)F)nc4ccc(Cl)cc34)CN(C(=O)C(F)F)C2)c(Cl)n1] | (M + H)⁺: 609.11 |

### Example 44

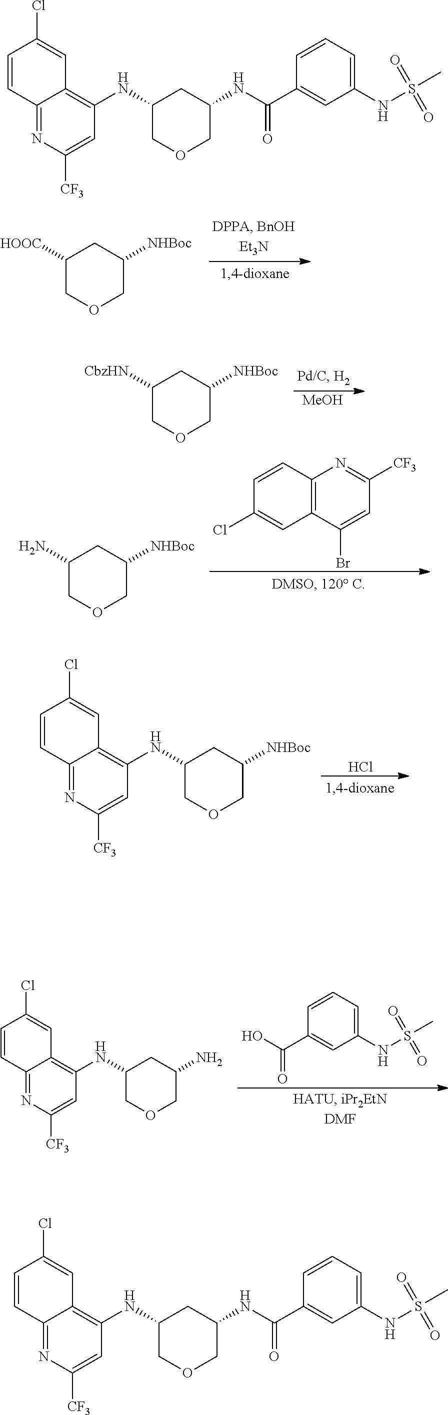

Step 44-1: cis-5-((tert-Butoxycarbonyl)amino)tetrahydro-2H-pyran-3-carboxylic acid (150.0 mg, 611.6 μmol) was dissolved in 1,4-dioxane. Triethylamine (256 μL, 1.835 mmol) and diphenyl phosphorazidate (263.6 μL, 1.223 mmol) were added. The reaction mixture was heated at 70° C. for 2 h. Benzyl alcohol (189.9 μL, 1.835 mmol) was added. The reaction mixture was heated at 70° C. for 16 h. The reaction mixture was concentrated and purified by silica gel chromatography to give benzyl tert-butyl (cis-tetrahydro-2H-pyran-3,5-diyl)dicarbamate (180 mg, 84.0%). ESI-MS m/z: 351.18 [M+H]⁺.

Step 44-2: Benzyl tert-butyl (cis-tetrahydro-2H-pyran-3,5-diyl)dicarbamate (180 mg, 514 μmol) was dissolved in methanol (2.57 mL). The solution was flushed with hydrogen. Pd/C (109 mg, 10% Wt, 103 μmol) was added. The reaction mixture was allowed to stir at rt for 2 h. The reaction mixture was filtered through a short celite plug. The organic solution was concentrated and used as crude.

Step 44-3:4-Bromo-6-chloro-2-(trifluoromethyl)quinoline (159.4 mg, 5.132 mmol) and tert-butyl (cis-5-aminotetrahydro-2H-pyran-3-yl) carbamate (111.0 mg, 5.132 mmol) was dissolved in DMSO (789.6 μL) and hunig's base (358 μL, 2.053 mmol) was added in a microwave vial. The reaction vessel was capped and heated at 130° C. in the microwave for 30 min. The reaction mixture was diluted with ethyl acetate. The organic layer was washed with brine 3 times and the residue was dried with magnesium sulfate and concentrated and purified by silica gel chromatography to give tert-butyl (cis-5-((6-chloro-2-(trifluoromethyl)quinolin-4-yl)amino)tetrahydro-2H-pyran-3-yl) carbamate (40 mg, 17%). ESI-MS m/z: 446.14 [M+H]⁺.

Step 44-4: tert-Butyl (cis-5-((6-chloro-2-(trifluoromethyl)quinolin-4-yl)amino)tetrahydro-2H-pyran-3-yl) carbamate (48.0 mg, 0.108 mmol) was dissolved in 1,4-dioxane (431 μL). HCl (269 μL, 4 molar, 1.08 mmol) was added. The reaction mixture was heated at 50° C. for 16 h. The reaction mixture was concentrated and used as crude.

Step 44-5: cis-N₃-(6-Chloro-2-(trifluoromethyl)quinolin-4-yl)tetrahydro-2H-pyran-3,5-diamine, HCl (21.1 mg, 55.2 μmol) and 3-(methylsulfonamido)benzoic acid (11.9 mg, 55.2 μmol) were dissolved in DMF (276 μL). Hunig's base (38.5 μL, 221 μmol) and HATU (31.5 mg, 82.8 μmol) were added. The reaction mixture was allowed to stir at room temperature for 2 h. The reaction mixture was diluted with ethyl acetate. The organic layer was washed with brine 3 times and dried by magnesium sulfate and concentrated and purified by reverse phase HPLC to give N-(cis-5-((6-chloro-2-(trifluoromethyl)quinolin-4-yl)amino)tetrahydro-2H-pyran-3-yl)-3-(methylsulfonamido)benzamide (10 mg, 33%). The racemic sample was purified by chiral HPLC (IC-5) to give Example 44 (2.7 mg, 27%) as a white powder. ESI-MS m/z: 543.98 [M+H]⁺.

### Example 45

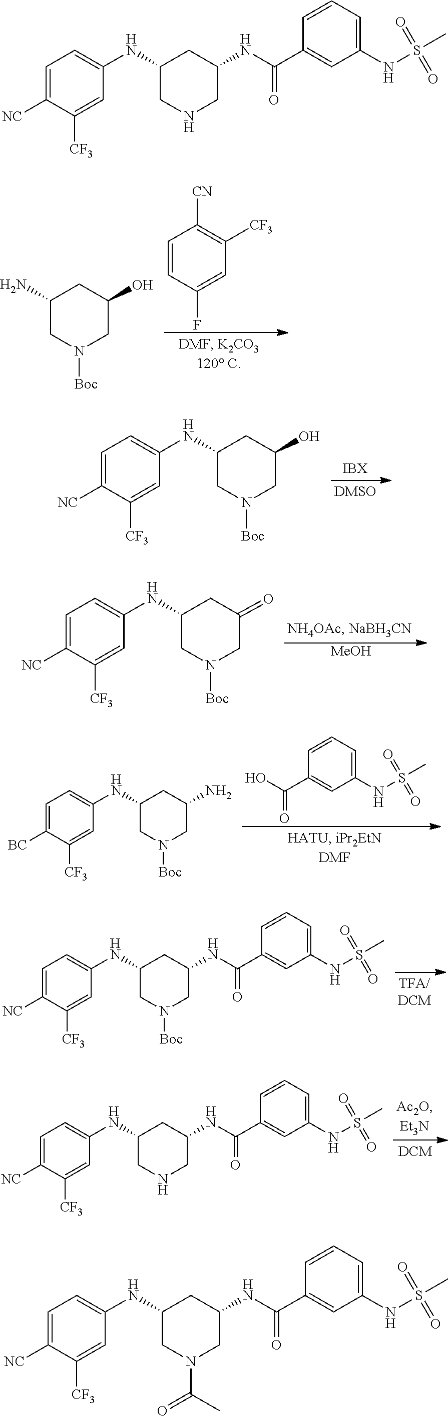

Step 45-1: To a solution of 4-fluoro-2-(trifluoromethyl)benzonitrile (1.152 g, 6.089 mmol) and tert-butyl (3R,5R)-3-amino-5-hydroxypiperidine-1-carboxylate (1.317 g, 6.089 mmol) in DMF (20.30 mL) at rt was added potassium carbonate (1.683 g, 12.18 mmol). Then the reaction mixture was heated 120° C. and kept for 3 h. The reaction was cooled to rt, quenched with NaHCO3 aq. solution. The reaction mixture was extracted with EtOAc 3 times. The combined organic phases were washed with water and brine, dried over Na₂SO₄, concentrated, and purified by silica gel chromatography eluting with EtOAc/hexanes to give the desired product (1.28 g, 55%) as a yellow solid. ESI-MS m/z: 386.06 [M+H]⁺.

Step 45-2: To a solution of the compound from Step 45-1 (1.28 g, 3.32 mmol) in DMSO (5.11 mL) at rt was added 2-iodoxybenzoic acid (1.86 g, 6.64 mmol), then the reaction mixture was stirred at rt for overnight. The reaction was quenched with Na₂S₂O₃ aq. solution. The reaction mixture was extracted with EtOAc 3 times. The combined organic phases were washed with water and brine, dried over Na2SO4, concentrated, and purified by silica gel chromatography eluting with EtOAc/hexanes to give the desired product (920 mg, 72%) as a yellow solid. ESI-MS m/z: 384.077 [M+H]⁺.

Step 45-3: To a solution of the compound from Step 45-2 (920 mg, 2.40 mmol) in MeOH (12.0 mL) at rt was added ammonium acetate (1.85 g, 24.0 mmol) and sodium cyanoborohydride (603 mg, 9.60 mmol), then the reaction mixture was stirred at rt for overnight. The reaction was quenched with NaHCO₃ aq. solution at rt. The reaction mixture was extracted with DCM for 3 times. The combined organic phases were dried over Na₂SO₄, concentrated, and purified by silica gel chromatography eluting with MeOH/CH₂Cl₂ to give the desired product (230 mg, 25%) as an off-white oil. ESI-MS m/z: 385.171 [M+H]⁺.

Step 45-4: To a solution of 3-(methylsulfonamido)benzoic acid (129 mg, 598 μmol) and the Compound from Step 45-3 (230 mg, 598 μmol) in DCM (3.0 mL) at rt was added N,N-diisopropylethylamine (232 mg, 1.80 mmol) and HATU (228 mg, 598 μmol), then the reaction mixture was stirred at rt for overnight. The reaction mixture was concentrated and purified by silica gel chromatography to give the desired product (301 mg, 87%) as a white solid. ESI-MS m/z: 582.146 [M+H]⁺.

Step 45-5: To a solution of the compound from Step 45-4 (67 mg, 0.12 mmol) in CH₂Cl₂ (0.58 mL) at 0° C. was added TFA (0.39 g, 3.5 mmol), then the reaction mixture was stirred at 0° C. for 15 min. The reaction was warmed to rt. The reaction mixture was concentrated and purified by silica gel chromatography eluting with EtOAc/hexanes to give the Example 45 (68 mg, 99%) as an off-white solid. ESI-MS m/z: 482.536 [M+H]⁺.

### Example 46

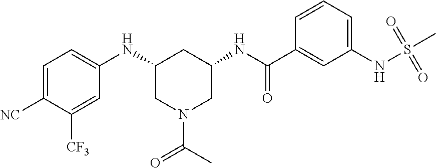 [SMILES: CC(=O)N1C[C@@H](NC(=O)c2cccc(NS(C)(=O)=O)c2)C[C@@H](Nc2ccc(C#N)c(C(F)(F)F)c2)C1]

To a solution of the compound from Step 45-5 (28 mg, 47 μmol) in DCM (1.6 mL) at rt was added triethylamine (14 mg, 0.14 mmol) and a solution of 0.2 N acetic anhydride (0.24 mL, 47 μmol) in DCM, then the mixture was kept at 25° C. for 1 h. The reaction mixture was Concentrated and purified by silica gel chromatography eluting with EtOAc/hexanes to give the Example 46 (21 mg, 85%) as an off-white solid. ESI-MS m/z: 524.043 [M+H]⁺.

### Example 47

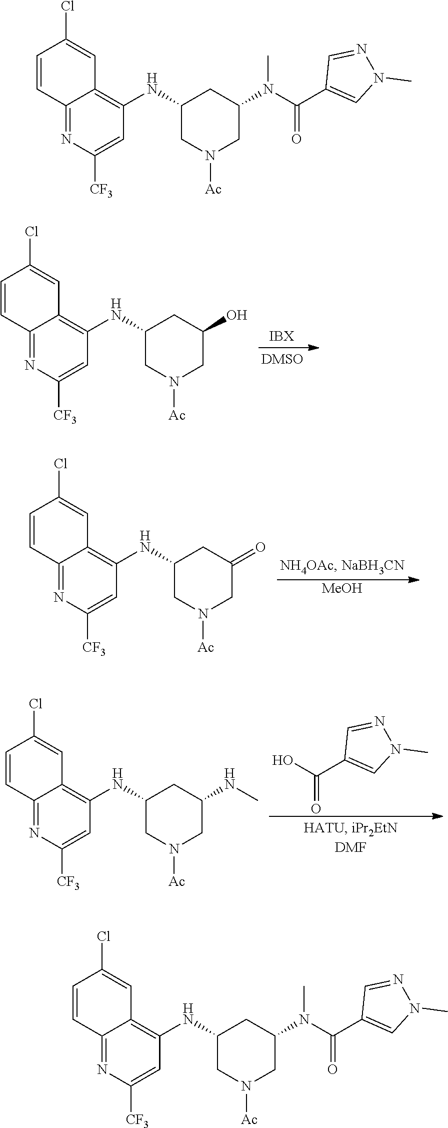

Step 47-1: To a solution of 1-((3R,5R)-3-((6-chloro-2-(trifluoromethyl) 74uinoline-4-yl)amino)-5-hydroxypiperidin-1-yl) ethan-1-one (722 mg, 1.86 mmol) in DMSO (2.86 mL) at room temperature was added 2-iodoxybenzoic acid (1.04 g, 3.72 mmol), then the reaction mixture was stirred at rt for overnight. The reaction was quenched with Na₂S₂O₃ aq. Solution. The reaction mixture was extracted with EtOAc for 3 times. The combined organic phases were washed with water and brine, dried over Na₂SO₄, concentrated, and purified by silica gel chromatography eluting with EtOAc/hexanes to give the desired product (573 mg, 80%) as a yellow solid. ESI-MS m/z: 386.298, 388.026 [M+H]⁺.

Step 47-2: To a solution of the compound from Step 47-1 (114 mg, 296 μmol) and methanamine hydrochloride (20.0 mg, 296 μmol) in MeOH (1.48 mL) at rt was added sodium cyanoborohydride (37.1 mg, 591 μmol), then the reaction mixture was stirred at rt for overnight. The reaction was quenched with NaHCO₃ aq. solution at rt. The reaction mixture was extracted with CH₂Cl₂ for 3 times. The combined organic phases were dried over Na₂SO₄, concentrated, and purified by silica gel chromatography eluting with MeOH/CH₂Cl₂ to give the desired product (42 mg, 25%) as an off-white oil. ESI-MS m/z: 401.102, 403.132 [M+H]⁺.

Step 47-3: To a solution of 1-methyl-1H-pyrazole-4-carboxylic acid (3.5 mg, 27 μmol) and the compound from Step 47-2 (11 mg, 27 μmol) in CH₂Cl₂ (1.4 mL) at rt was added N-ethyl-N-isopropylpropan-2-amine (11 mg, 82 μmol) and HATU (10 mg, 27 μmol), then the reaction mixture was stirred at rt for overnight. The reaction mixture was concentrated and purified by flash column to give the Example 47 (8.9 mg, 64%) as a white solid. ESI-MS m/z: 509.128, 511.003 [M+H]⁺.

The following examples were prepared using procedures similar to those described above:

| Example | Structure | ESI-MS |
|---|---|---|
| 48 | 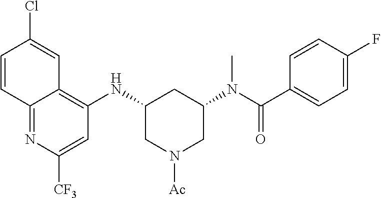 [SMILES: CC(=O)N1C[C@H](Nc2cc(C(F)(F)F)nc3ccc(Cl)cc23)C[C@H](N(C)C(=O)c2ccc(F)cc2)C1] | (M + H)⁺: 523.102 |

### Example 49

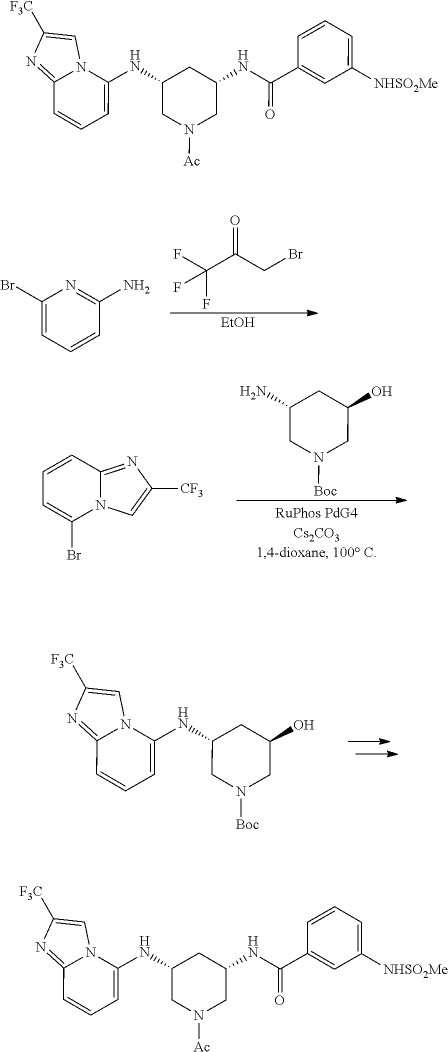

Step 49-1:6-Bromopyridin-2-amine (10.0 g, 57.8 mmol) and 3-bromo-1,1,1-trifluoroacetone (12 mL, 116 mmol) were dissolved in ethanol (100 mL) and the mixture was heated under reflux for 4 days. The precipitated solid was collected by filtration and dissolved in dichloromethane. The solution was washed with saturated aqueous sodium bicarbonate solution and dried over anhydrous sodium sulfate and the solvent was evaporated under reduced pressure to give the desired compound (11.2 g, 74%) which was used directly in the next step.

Step 49-2: In a 40 mL vial, tert-butyl (3R,5R)-3-amino-5-hydroxypiperidine-1-carboxylate (300 mg, 1.4 mmol), 5-bromo-2-(trifluoromethyl) imidazo[1,2-a]pyridine (386 mg, 1.46 mmol), cesium carbonate (1.36 g, 4.2 mmol), and RuPhos PdG4 (118 mg, 140 μmol) were dissolved in 1,4-Dioxane (7.0 mL). The vial was sealed and heated to 100° C. for 4 h. The reaction was allowed to cool and water was added. The aqueous layer was washed with EtOAc and the combined organic layer was washed with brine before drying over Na₂SO₄ and concentrating under reduced pressure. The mixture was purified by silica gel chromatography eluting with EtOAc/Hex to furnish the desired compound (260 mg, 47%). ESI-MS m/z: 401.40 [M+H]⁺.

Example 49 was synthesized with the product form Step 49-2 via an analogous way to synthesize Example 1. ESI-MS m/z: 581.28 [M+H]⁺.

The following examples were prepared using procedures similar to those described above:

| Example | Structure | ESI-MS |
|---|---|---|
| 50 | 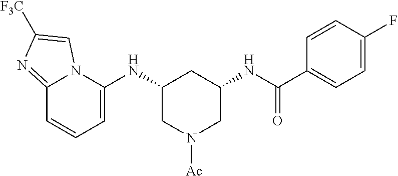 [SMILES: CC(=O)N1C[C@@H](NC(=O)c2ccc(F)cc2)C[C@@H](Nc2cccc3nc(C(F)(F)F)cn23)C1] | (M + H)⁺: 464.16 |

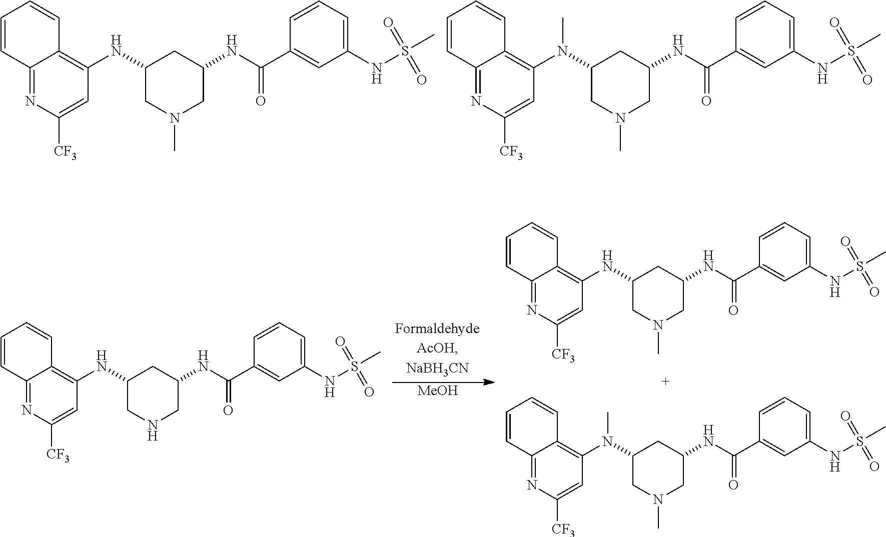

N-(cis-5-(2-(trifluoromethyl)quinolin-4-yl)amino)piperidin-3-yl)-3-(methylsulfonamido)benzamide (121 mg, 253 μmol) was dissolved in methanol (2.53 mL) and formaldehyde (188 μL, 37% Wt, 2.53 mmol) was added. The reaction mixture was allowed to stir at room temperature for 10 min. Acetic acid (43.4 μL, 758 μmol) and sodium cyanoborohydride (47.6 mg, 758 μmol) were added and the reaction mixture was allowed to stir at room temperature for 1 h. The reaction mixture was diluted with water and ethyl acetate. The aqueous layer was extracted with ethyl acetate and the combined organic layers were dried by magnesium sulfate and concentrated and purified by reverse phase HPLC to give Example 51 (10 mg, 9% yield), ESI-MS m/z: 522.187 [M+H]⁺ and Example 52 (10 mg, 9% yield), ESI-MS m/z: 536.095 [M+H]⁺.

### Example 53

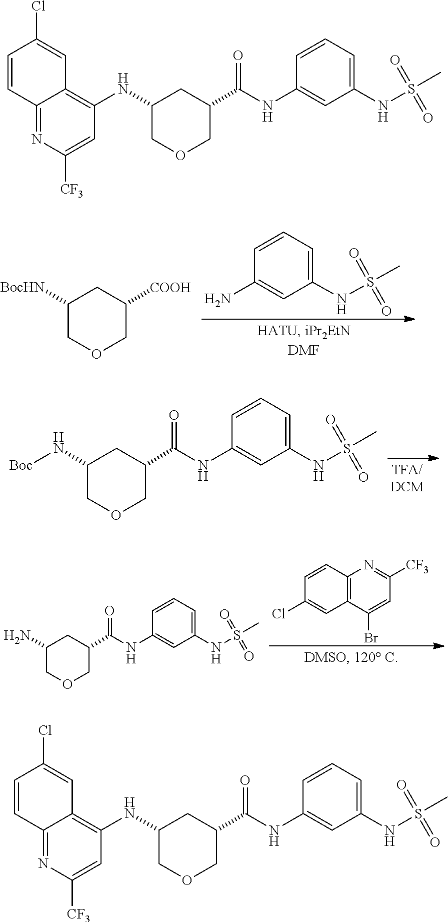

Step 53-1: cis-5-((tert-Butoxycarbonyl)amino)tetrahydro-2H-pyran-3-carboxylic acid (100 mg, 408 μmol) in DMF (4.08 mL) was added N-(3-aminophenyl) methanesulfonamide (75.9 mg, 408 μmol), hunig base (0.36 mL, 2.04 mmol), and HATU (233 mg, 612 μmol) at room temperature. The reaction mixture was allowed to stir at room temperature for 5 h. The reaction mixture was diluted with ethyl acetate and washed with brine. The organic layer was dried with magnesium sulfate and concentrated and purified by silica gel chromatography to give tert-butyl (cis-5-((3-(methylsulfonamido)phenyl) carbamoyl)tetrahydro-2H-pyran-3-yl) carbamate (150 mg, 89.0%). ESI-MS m/z: 309.92 [M+H]⁺.

Step 53-2: tert-butyl (cis-5-((3-(Methylsulfonamido)phenyl) carbamoyl)tetrahydro-2H-pyran-3-yl) carbamate (150 mg, 363 μmol) was dissolved in DCM (1.2 mL) and TFA (827 mg, 559 μL, 7.26 mmol) was added. The reaction mixture was allowed to stir at room temperature for 16 h. The reaction mixture was concentrated and used as crude.

Step 53-3:4-Bromo-6-chloro-2-(trifluoromethyl)quinoline (154 mg, 495 μmol) and cis-5-amino-N-(3-(methylsulfonamido)phenyl)tetrahydro-2H-pyran-3-carboxamide (155 mg, 495 μmol) was dissolved in DMSO (761 μL) and hunig's base (256 mg, 1.98 mmol) was added in a microwave vial. The reaction vessel was capped and heated at 130° C. in the microwave for 30 min. The reaction mixture was diluted with ethyl acetate and washed with brine 3 times. The organic layer was dried with magnesium sulfate and concentrated and purified by reverse phase HPLC to give the Example 53 (30 mg, 11%) as a white power, ESI-MS m/z: 543.931 [M+H]⁺.

### Example 54

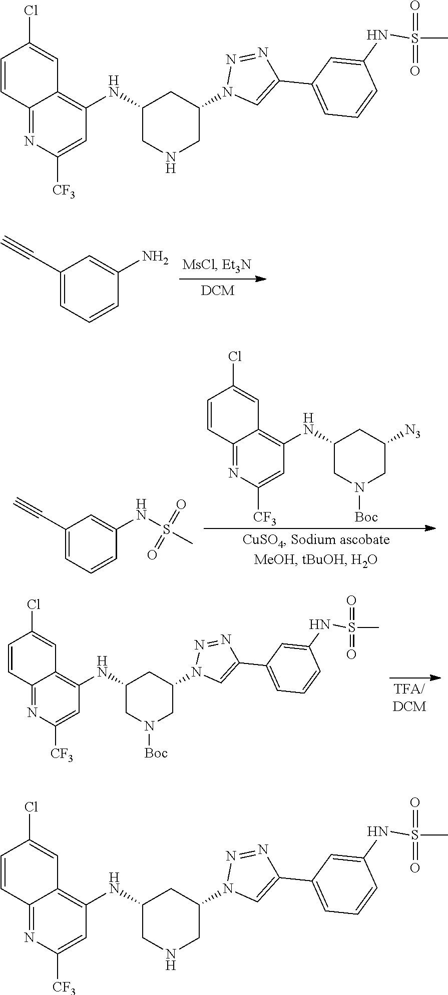

Step 54-1:3-Ethynylaniline (422 mg, 3.60 mmol) in DCM (18.0 mL) was added triethylamine (729 mg, 7.20 mmol) and mesyl-C₁ (495 mg, 4.32 mmol) at room temperature. The reaction mixture was allowed to stir at room temperature for 5 h. The reaction mixture was diluted with ethyl acetate and washed with brine. The organic layer was dried with magnesium sulfate and concentrated and purified by silica gel chromatography to give N-(3-ethynylphenyl) methanesulfonamide (298 mg, 42.4%). ESI-MS m/z: 196.04 [M+H]⁺.

Step 54-2: tert-Butyl (3S,5R)-3-azido-5-((6-chloro-2-(trifluoromethyl)quinolin-4-yl)amino)piperidine-1-carboxylate (70 mg, 0.15 mmol) and N-(3-ethynylphenyl) methanesulfonamide (38 mg, 0.19 mmol) were dissolved in methanol (1.2 mL), t-BuOH (1.2 mL) and water (0.59 mL). (R)-5-((S)-1,2-dihydroxyethyl)-3,4-dihydroxyfuran-2 (5H)-one (10 mg, 59 μmol) and copper (II) sulfate (2.4 mg, 15 μmol) were added. The resulting solution was allowed to stir at room temperature for 16 h. The reaction mixture was diluted with ethyl acetate and water. The aqueous layer was extracted with ethyl acetate 3 times. The combined organic layers were dried with magnesium sulfate and concentrated and purified by silica gel chromatography to give tert-butyl (3R,5S)-3-((6-chloro-2-(trifluoromethyl)quinolin-4-yl)amino)-5-(4-(3-(methylsulfonamido)phenyl)-1H-1,2,3-triazol-1-yl)piperidine-1-carboxylate (78 mg, 79%). ESI-MS m/z: 666.18 [M+H]⁺.

Step 545-3: tert-Butyl (3R,5S)-3-((6-chloro-2-(trifluoromethyl)quinolin-4-yl)amino)-5-(4-(3-(methylsulfonamido)phenyl)-1H-1,2,3-triazol-1-yl)piperidine-1-carboxylate (78 mg, 0.12 mmol) was dissolved in DCM (1 mL) and TFA (0.73 g, 6.4 mmol) was added. The reaction mixture was allowed to stir at room temperature for 16 h. The reaction mixture was concentrated and used as crude. A small portion was purified by reverse phase HPLC to give Example 54, ESI-MS m/z: 566.012 [M+H]⁺.

### Example 55

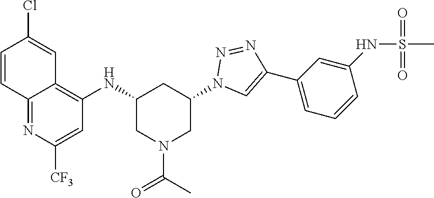 [SMILES: CC(=O)N1C[C@H](Nc2cc(C(F)(F)F)nc3ccc(Cl)cc23)C[C@H](n2cc(-c3cccc(NS(C)(=O)=O)c3)nn2)C1]

N-(3-(1-((3S,5R)-5-((6-Chloro-2-(trifluoromethyl)quinolin-4-yl)amino)piperidin-3-yl)-1H-1,2,3-triazol-4-yl)phenyl) methanesulfonamide (40 mg, 71 μmol) in DMF (0.35 mL) from the last step was added acetic acid (6.4 mg, 0.11 mmol), Hunig's base (0.096 mL, 0.55 mmol) and HATU (40 mg, 0.11 mmol) at room temperature. The reaction mixture was allowed to stir at room temperature for 5 h. LCMS shows full conversion to the desired product. The reaction mixture was diluted with ethyl acetate and washed with brine. The organic layer was dried with magnesium sulfate and concentrated and purified by reverse phase HPLC to give Example 55 (10 mg, 23%) as a white powder, ESI-MS m/z: 608.301 [M+H]⁺.

### Example 71

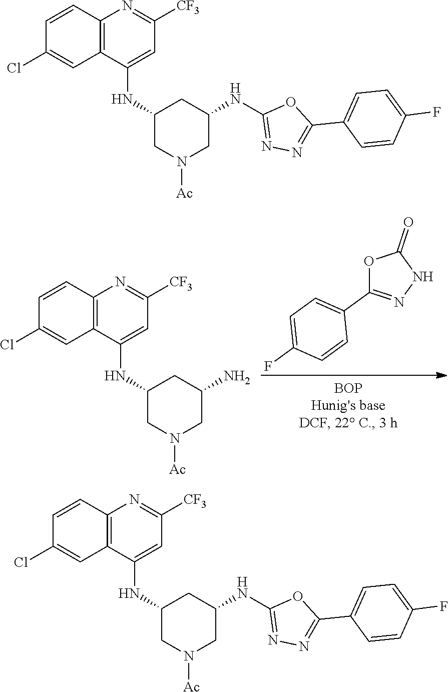

The 5-(4-fluorophenyl)-1,3,4-oxadiazol-2 (3H)-one (18 mg, 99 μmol) was added to a vial. DMF (1.3 mL) was added to the vial. hunig's base (39 mg, 0.30 mmol), and 1-((3S,5R)-3-amino-5-((6-chloro-2-(trifluoromethyl)quinolin-4-yl)amino)piperidin-1-yl) ethan-1-one (50 mg, 0.13 mmol) were added sequentially to this solution. BOP (48 mg, 0.11 mmol) was added to the mixture. The vial was sealed and stirred at room temperature for 3 h. Water was added, and the aqueous layer was washed with EtOAc. The combined organic layer was dried over MgSO₄ and concentrated under reduced pressure. The reaction mixture was purified by prep-HPLC to give Example 71 (23 mg, 42%). ESI-MS m/z: 549.80 [M+H]⁺.

### Example 125

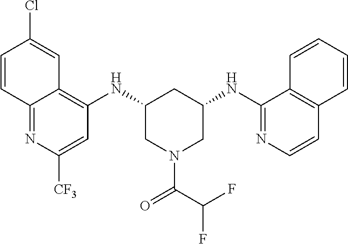 [SMILES: O=C(C(F)F)N1C[C@@H](Nc2nccc3ccccc23)C[C@@H](Nc2cc(C(F)(F)F)nc3ccc(Cl)cc23)C1]

A solution of 1-((3S,5R)-3-amino-5-((6-chloro-2-(trifluoromethyl)quinolin-4-yl)amino)piperidin-1-yl)-2,2-difluoroethan-1-one (30 mg, 1.0 Eq, 71 μmol) in THF (0.2 mL) was treated with DIPEA (34 mg, 46 μL, 3.75 eq, 0.27 mmol). A solution of isoquinoline 2-oxide (10 mg, 1 Eq, 71 μmol) and PyBrop (43 mg, 1.3 Eq, 92 μmol) was added after 10 mins. After being stirred at 60° C. for 2 h, reaction mixture was conc., purified by Prep-HPLC to give title compound (19 mg, 35 μmol, 49%). ESI-MS m/z: 550.24 [M+H]⁺.

### Example 126

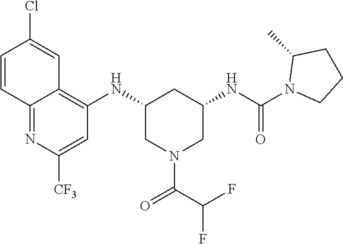 [SMILES: C[C@@H]1CCCN1C(=O)N[C@H]1C[C@@H](Nc2cc(C(F)(F)F)nc3ccc(Cl)cc23)CN(C(=O)C(F)F)C1]

A solution of 1-((3S,5R)-3-amino-5-((6-chloro-2-(trifluoromethyl)quinolin-4-yl)amino)piperidin-1-yl)-2,2-difluoroethan-1-one (30 mg, 1.0 eq, 71 μmol) in MeCN (0.2 mL) was treated with CDI (13 mg, 1.1 eq, 78 μmol), a solution of (R)-2-methylpyrrolidine (6.0 mg, 1 Eq, 71 μmol) in MeCN (0.2 mL) and DBU (27 mg, 27 μL, 2.5 eq, 0.18 mmol) was added after 10 mins. It was stirred at 60° C. for 2 h, purified by prep-HPLC to give title compound (9.2 mg, 17 μmol, 24%). ESI-MS m/z: 534.35 [M+H]⁺.

| Example | Structure | ESI-MS |
|---|---|---|
| 127 | 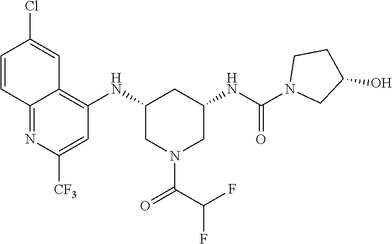 [SMILES: O=C(N[C@H]1C[C@@H](Nc2cc(C(F)(F)F)nc3ccc(Cl)cc23)CN(C(=O)C(F)F)C1)N1CC[C@H](O)C1] | (M + H)⁺: 536.12 |
| 128 | 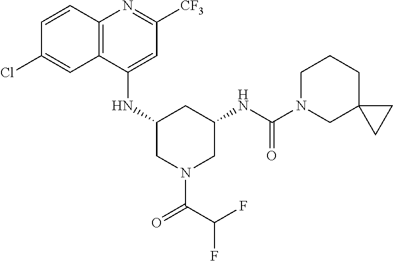 [SMILES: O=C(N[C@H]1C[C@@H](Nc2cc(C(F)(F)F)nc3ccc(Cl)cc23)CN(C(=O)C(F)F)C1)N1CCCC2(CC2)C1] | (M + H)⁺: 560.18 |
| 129 | 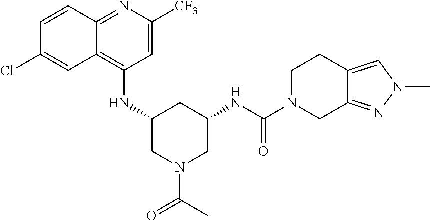 [SMILES: CC(=O)N1C[C@@H](NC(=O)N2CCc3cn(C)nc3C2)C[C@@H](Nc2cc(C(F)(F)F)nc3ccc(Cl)cc23)C1] | (M + H)⁺: 550.22 |

### Example 130

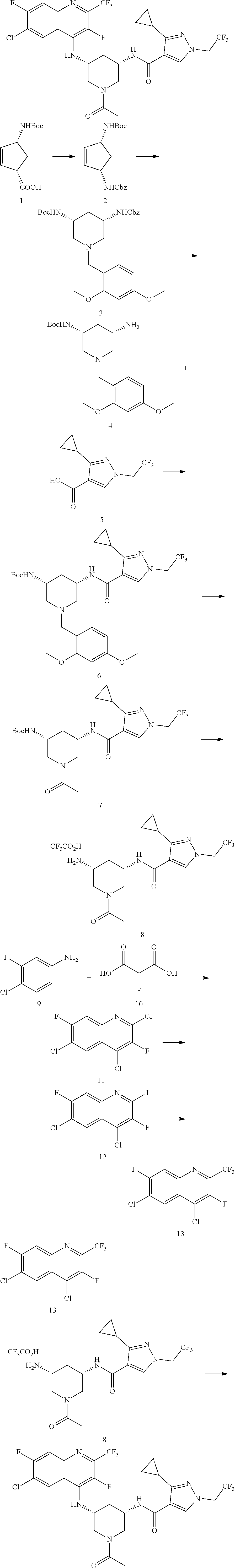

Compound 1 (20.00 g, 88.00 mmol), Diphenylphosphoryl azide (31.48 g, 24.67 mL, 1.3 eq, 114.4 mmol) and triethylamine (13.36 g, 18.4 mL, 1.5 eq, 132.0 mmol) were dissolved in toluene (200 mL) and stirred at rt for 10 min, then moved to 90° C. and hold for 15 min. Benzyl alcohol (12.37 g, 11.90 mL, 1.3 eq, 114.4 mmol) was added and the reaction was stirred at 90° C. for 1 h, then cool to rt. Concentrated and purified by CombiFlash (EA/c-Hex: 10˜25%) to give 2 (20.2 g, y. 69.1%) as a pale yellow oil.

2 (10.00 g, 30.08 mmol) was dissolved in MeOH (200 mL) and the solution was cooled to −78° C. The solution was flushed with ozone until the color changed to blue. Ozone was removed and DMS (9.346 g, 11.1 mL, 5 eq, 150.4 mmol) was added. The reaction mixture was allowed to stir at rt for 50 min, then cooled to 0° C. (2,4-dimethoxyphenyl) methanamine (5.684 g, 5.03 mL, 1.13 eq, 34.00 mmol) and acetic acid (7.226 g, 6.889 mL, 4 eq, 120.3 mmol) were added, stirred for 5 min, then sodium cyanoborohydride (6.806 g, 3.6 eq, 108.3 mmol) was added in small portions over 15 min. The reaction mixture was allowed to stir at 60° C. for 18 h. Cooled to rt, half of the volume was removed by rotavap, quenched by portion wise adding to ˜200 mL stirring sat. NaHCO₃ solution, and then allow to stir at rt for 10 min. The aqueous layer was extracted with DCM 3 times. The combined organic layers were concentrated and purified by CombiFlash (EA/c-Hex: 10˜30%) to give 3 (7.22 g, y. 48.0%) as a white solid. ESI-MS m/z: 500.72 [M+H]⁺.

A mixture of 3 (5.00 g, 10.0 mmol) and Pd/C (1.06 g, 10% Wt, 0.1 eq, 1.00 mmol) in MeOH (20 mL) was stirred under H₂ for 16 h, it was then filtered through celite. Concentrated to 4 (3.65 g, y. 100%).

To a 20 mL vial were added 4 (816.2 mg, 1.2 eq, 2.233 mmol), 5 (435.8 mg, 1.861 mmol), HATU (1.061 g, 1.5 eq, 2.791 mmol), DCM (7.0 mL), and hunig'sbase (601.3 mg, 810 μL, 2.5 eq, 4.652 mmol) respectively and the turbid orange mixture was stirred at rt for 1.5 h. The reaction was complete by LC/MS and TLC. Diluted with DCM, washed with 10% citric acid, sat. NaHCO₃, and brine sequentially. Dried, filtered, concentrated and purified by CombiFlash (Ace/c-Hex: 0˜50%) to give 6 (885.0 mg, y. 81.76%) as a white solid. ESI-MS m/z: 582.29 [M+H]⁺.

To a 25 mL round-bottomed flask were added 6 (450 mg, 774 μmol), DCM (3 mL), triethylamine (235 mg, 324 μL, 3 eq, 2.32 mmol), and trifluoroacetic anhydride (650 mg, 437 μL, 4 eq, 3.09 mmol) and the reaction was stirred at rt for 1.5 h. Concentrated to remove solvent. MeOH (4 mL) was added followed by a solution of sodium hydroxide (279 mg, 9 eq, 6.96 mmol) in 1 mL of water and the solution was stirred at rt for 40 min. Water (12 mL) was added and white precipitates formed. The white solid was filtered, washed with water, and transferred to a 50 mL round-bottomed flask by dissolving the solid in DCM/MeOH. Concentrated to dryness in vacuo to give a white solid 326.1 mg. DCM (5 mL), triethylamine (157 mg, 216 μL, 2 eq, 1.55 mmol), and acetic anhydride (158 mg, 146 μL, 2 eq, 1.55 mmol) were added respectively and the reaction was stirred at rt for 25 min. Diluted with DCM, washed with sat. NaHCO₃ and brine. Dried, filtered, concentrated to give 7 (356.2 mg, y. 97.2%) as a crude orange solid. ESI-MS m/z: 474.23 [M+H]⁺.

To a 40 mL vial containing 7 (350 mg, 739 μmol) were added DCM (3 mL) and TFA (2.2 g, 1.5 mL, 26 eq, 19 mmol) and the solution was stirred at rt for 30 min. The reaction was complete by TLC and LC/MS. Concentrated and lyophilized to give 8 (482 mg, y. quan.) as a light brown solid. ESI-MS m/z: 374.18 [M+H]⁺.

To a 100 mL round-bottomed flask were 9 (2.0 g, 1 eq, 14 mmol), 10 (2.0 g, 1.2 eq, 16 mmol), and phosphorus oxychloride (32 g, 19 mL, 15 eq, 0.21 mol) and the reaction was heated at 100° C. for 24 h. Cooled to rt. Poured into ice and some yellow solid formed. Slowly basified with aq. NaOH solution (40 g in 200 mL of water) to basic when cooled with ice-water bath. Filtered via a Buchner funnel to collect the yellow solid which was purified by CombiFlash (80 g SiO₂, EA/c-Hex: 0˜30%) to give 11 (1.14 g, y. 31%) as a yellow solid. ESI-MS m/z: 265.97, 267.97 [M+H]⁺. 1H NMR (400 MHZ, DMSO) δ 8.42 (d, J=7.7 Hz, 1H), 8.21 (d, J=9.9 Hz, 1H).

To a 50 mL round-bottomed flask were added 11 (500 mg, 1.86 mmol), sodium iodide (837 mg, 3.0 eq, 5.59 mmol), ACN (9.31 mL), and acetyl chloride (190 mg, 172 μL, 1.3 eq, 2.42 mmol) and the yellow suspension turned orange immediately. The r×n was stirred at rt for 45 min. Diluted with EtOAc, washed with Sat. NaHCO₃ and brine respectively. Dried, filtered, concentrated and purified by CombiFlash (40 g SiO₂, EA/c-Hex: 0˜20%) to give 12 (395 mg, y. 60%) as a white solid. ESI-MS m/z: 359.87, 361.86 [M+H]⁺1H NMR (400 MHZ, DMSO) δ 8.35 (d, J=7.7 Hz, 1H), 8.18 (d, J=10 Hz, 1H). ¹⁹F NMR (376 MHz, DMSO) δ −104.93 (d, J=7.4 Hz), −111.91 (d, J=7.3 Hz).

To a 2-dram vial were added 12 (100.4 mg, 279.0 μmol), (1,10-phenanthroline)(trifluoromethyl)copper(I) (185.60 mg, 2.127 eq, 593.43 μmol), and DMF (1.5 mL) and the mixture was heated at 70° C. for 1 h 45 min. Cooled to rt, diluted with EtOAc, washed with water twice and brine once. Dried, filtered, concentrated and purified by CombiFlash (12 g SiO₂, EA/c-Hex: 0˜20%) to give 13 (50 mg, y. 59%). 1H NMR (400 MHZ, DMSO) δ 8.54 (d, J=7.6 Hz, 1H), 8.41 (d, J=9.8 Hz, 1H). ¹⁹F NMR (376 MHZ, DMSO) δ −65.00 (d, J=15.0 Hz), −110.68 (d, J=6.8 Hz), −129.06 (qd, J=14.8, 6.8 Hz).

To a 2 mL vial were added 13 (43.9 mg, 145 μmol), 8 (70.8 mg, 1 eq, 145 μmol), DMSO (0.5 mL) and Hunig's base (93.9 mg, 127 μL, 5 eq, 727 μmol) respectively and the reaction was heated at 90° C. for 12 h. Cooled to rt, diluted with ethyl acetate and washed with brine 3 times. Dried, filtered, concentrated and purified by CombiFlash (12 g SiO₂, Ace/c-Hex: 0˜50%) to give title compound (20 mg, y. 22%). ESI-MS m/z: 639.15 [M+H]⁺.

| Example | Structure | ESI-MS |
|---|---|---|
| 131 | 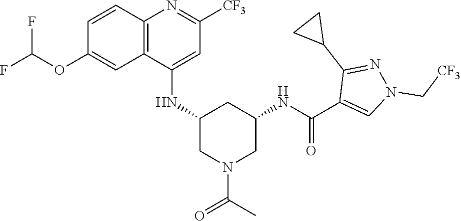 [SMILES: CC(=O)N1C[C@@H](NC(=O)c2cn(CC(F)(F)F)nc2C2CC2)C[C@@H](Nc2cc(C(F)(F)F)nc3ccc(OC(F)F)cc23)C1] | (M + H)⁺: 635.15 |
| 132 | 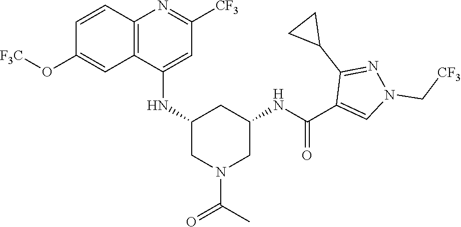 [SMILES: CC(=O)N1C[C@@H](NC(=O)c2cn(CC(F)(F)F)nc2C2CC2)C[C@@H](Nc2cc(C(F)(F)F)nc3ccc(OC(F)(F)F)cc23)C1] | (M +H)⁺: 653.20 |
| 133 | 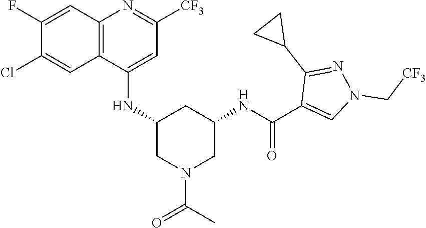 [SMILES: CC(=O)N1C[C@@H](NC(=O)c2cn(CC(F)(F)F)nc2C2CC2)C[C@@H](Nc2cc(C(F)(F)F)nc3cc(F)c(Cl)cc23)C1] | (M + H)⁺: 621.15 |
| 134 | 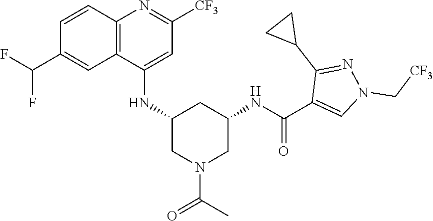 [SMILES: CC(=O)N1C[C@@H](NC(=O)c2cn(CC(F)(F)F)nc2C2CC2)C[C@@H](Nc2cc(C(F)(F)F)nc3ccc(C(F)F)cc23)C1] | (M + H)⁺: 619.25 |
| 135 | 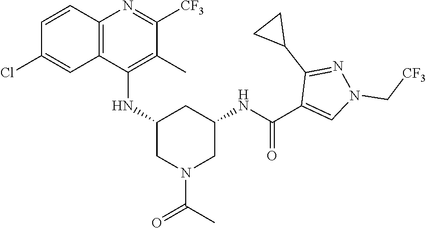 [SMILES: CC(=O)N1C[C@@H](NC(=O)c2cn(CC(F)(F)F)nc2C2CC2)C[C@@H](Nc2c(C)c(C(F)(F)F)nc3ccc(Cl)cc23)C1] | (M + H)⁺:617.25 |
| 136 | 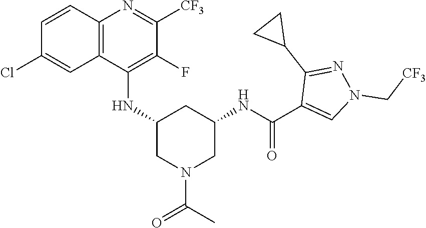 [SMILES: CC(=O)N1C[C@@H](NC(=O)c2cn(CC(F)(F)F)nc2C2CC2)C[C@@H](Nc2c(F)c(C(F)(F)F)nc3ccc(Cl)cc23)C1] | (M + H)⁺: 621.20 |
| 137 | 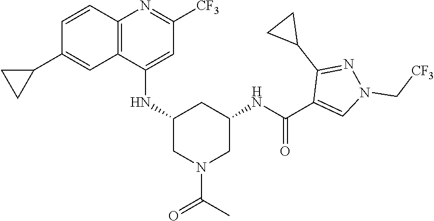 [SMILES: CC(=O)N1C[C@@H](NC(=O)c2cn(CC(F)(F)F)nc2C2CC2)C[C@@H](Nc2cc(C(F)(F)F)nc3ccc(C4CC4)cc23)C1] | (M + H)⁺: 609.20 |
| 138 | 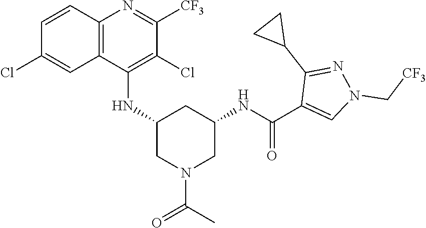 [SMILES: CC(=O)N1C[C@@H](NC(=O)c2cn(CC(F)(F)F)nc2C2CC2)C[C@@H](Nc2c(Cl)c(C(F)(F)F)nc3ccc(Cl)cc23)C1] | (M + H)⁺: 637.12 |
| 139 | 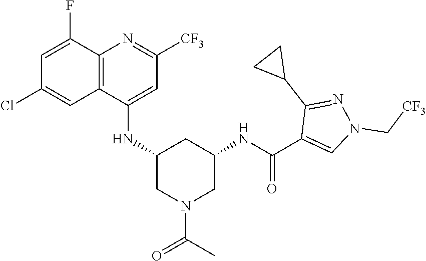 [SMILES: CC(=O)N1C[C@@H](NC(=O)c2cn(CC(F)(F)F)nc2C2CC2)C[C@@H](Nc2cc(C(F)(F)F)nc3c(F)cc(Cl)cc23)C1] | (M + H)⁺: 621.15 |

### Example 140

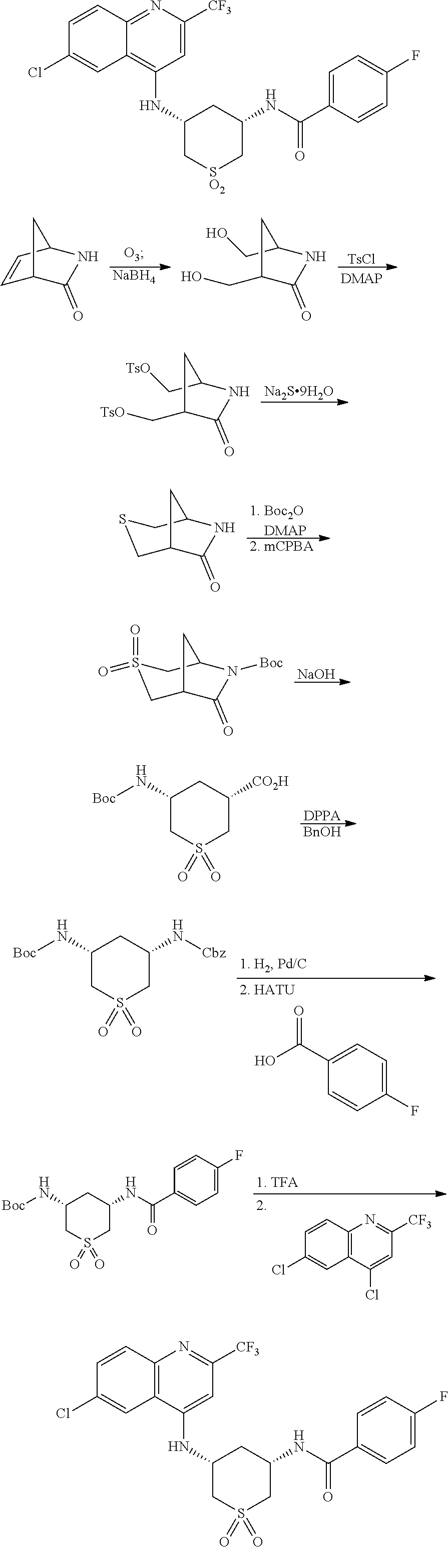

In a 100 mL rbf, (1S,4R)-2-azabicyclo[2.2.1]hept-5-en-3-one (1000 mg, 9.163 mmol) was dissolved in MeOH (45 mL). The solution was cooled to ˜−60° C. Ozone was bubbled through the solution until a blue color persisted and by TLC (50 μL MeOH in 10 mL EtOAc) no SM was present. The excess ozone was blow out by bubbling nitrogen through the reaction. Solution allowed to warm to ˜−30° C. and sodium borohydride (350 mg, 9.163 mmol) was added slowly. Reaction allowed to warm to −5 C over 1 h. Reaction was acidified using AmberChrom 50WX4 to pH=˜ 5. Solution filtered and concentrated. MeOH added and solution concentrated. Small amount of Et₂O added and solution concentrated under reduced pressure to furnish the title compound (1.3 g, 98%) as a light peach solid, which was taken forward without further purification.

In a vial, (3S,5R)-3,5-bis(hydroxymethyl) pyrrolidin-2-one (2.0 g, 13.8 mmol) and DMAP (842 mg, 6.89 mmol) were dissolved in Pyridine (20 mL). TsCl (5.52 g, 28.9 mmol) was added and the reaction was allowed to stir at room temperature for 4 h. Water was added followed by 1M HCl. The aqueous layer was washed with EtOAc. The combined organic layer was washed with brine and dried over MgSO4 before concentrating. The mixture was purified by column chromatography (070% EtOAc/cHex) to give the title compound (3.53 g, 56%) ESI-MS m/z: 454.45 [M+H].

In a round bottom flask equipped with a condenser, ((2R,4S)-5-oxopyrrolidine-2,4-diyl)bis(methylene)bis(4-methylbenzenesulfonate) (3.53 mg, 7.78 mmol) and sodium sulfide nonahydrate (2.2 g, 9.34 mmol) were dissolved in DMF (52 mL). The reaction was heated to 90° C. for 1 h. Reaction cooled to room temperature and water added and aqueous layer washed with EtOAc. Combined organic layer concentrated and purified by column chromatography (0-7% EtOAc/cHex) to give the title compound (550 mg, 49%). ESI-MS m/z: 143.86 [M+H].

In a vial, (1R,5R)-3-thia-6-azabicyclo[3.2.1]octan-7-one (400 mg, 2.79 mmol) and DMAP (68 mg, 0.56 mmol) were dissolved in DCM (6 mL). Et₃N 700 mg, 7.0 mmol) was added followed by Boc₂O (884 mg, 4.0 mmol). The reaction was allowed to stir overnight. Water was added and the aqueous layer was washed with DCM. The combined organic layer was dried over MgSO₄ and concentrated under reduced pressure. Reaction mixture purified by column chromatography (0-50% EtOAc/cHex) to give the title compound (420 mg, 62%). ESI-MS m/z: 187.91 [M+H-tBu].

In a vial, tert-butyl (1R,5R)-7-oxo-3-thia-6-azabicyclo[3.2.1]octane-6-carboxylate (200 mg, 0.82 mmol) was dissolved in DCM (2 mL). mCPBA (426 mg, 70% Wt, 1.73 mmol) was added slowly. Warning reaction is exothermic. The reaction mixture was allowed to stir overnight. The reaction was quenched upon addition of Na₂SCO₃ following by sat. aq. NaHCO₃. The aqueous layer was washed with DCM and the combined organic layer was washed with brine. The organic layer was dried over MgSO₄ before concentrating under reduced pressure to give the title compound as a white solid (220 mg, 97%).

To a stirred solution of tert-butyl (1R,5R)-7-oxo-3-thia-6-azabicyclo[3.2.1]octane-6-carboxylate 3,3-dioxide (100 mg, 0.36 mmol) in THF (1.0 mL) was added NaOH (0.9 mL, 1 molar, 0.9 mmol) and the mixture was stirred at 40° C. for 20 h. The reaction mixture was cooled to 0° C. and acidified to pH 3.5 with 1N hydrochloric acid. The mixture was extracted with DCM and the combined extracts were washed with water, brine and dried Na₂SO₄. The solvent was evaporated under reduced pressure to give the title compound (65 mg, 61%).

In a vial, (3R,5R)-5-((tert-butoxycarbonyl)amino)tetrahydro-2H-thiopyran-3-carboxylic acid 1,1-dioxide (60 mg, 0.20 mmol), diphenyl phosphorazidate (57 μL, 0.27 mmol) and triethylamine (43 μL, 0.31 mmol) were dissolved in Toluene (1.0 mL) and heated at 90° C. for 1 h. benzyl alcohol (43 μL, 0.41 mmol) was added and heated at 90° C. for 16 h. After cooling to room temperature, the reaction mixture was concentrated and purified by column chromatography to give the title compound (60 mg, 73%). ESI-MS m/z: 298.94 [M+H-Boc].

In a vial, benzyl tert-butyl ((3R,5S)-1,1-dioxidotetrahydro-2H-thiopyran-3,5-diyl)dicarbamate (60 mg, 0.15 mmol) was dissolved in MeOH (1.5 mL). Pd/C (16 mg, 0.15 mmol) was added and the vial was sparged with H₂. The reaction was allowed to stir at room temperature under an atmosphere of H₂ for 4 h. The reaction was filtered through celite and concentrated to give the desired product (30 mg, 75%).

In a vial, tert-butyl ((3R,5S)-5-amino-1,1-dioxidotetrahydro-2H-thiopyran-3-yl) carbamate (60 mg, 0.23 mmol), 4-fluorobenzoic acid (35 mg, 0.25 mmol), and HATU (104 mg, 0.27 mmol) were dissolved in DMF (2.3 mL). Hunig's base (120 μL, 0.68 mmol) was added and the reaction was allowed to stir overnight. Water was added and the aqueous layer was washed with EtOAc. The combined organic layer was dried over MgSO₄ before concentrating. The mixture was purified by column chromatography (0-100% EtOAc/cHex) to give the desired product (50 mg, 57%). ESI-MS m/z: 330.97 [M+H-tBu].

In a vial, tert-butyl ((3R,5S)-5-(4-fluorobenzamido)-1,1-dioxidotetrahydro-2H-thiopyran-3-yl) carbamate (50 mg, 0.13 mmol) was dissolved in DCM (1.3 mL). TFA (50 μL, 0.65 mol) was added and the reaction was allowed to stir overnight. The reaction was concentrated under reduced pressure to give the desired product (30 mg, 81%), which was taken forward without further purification. ESI-MS m/z: 286.94 [M+H].

In a vial, N-((3S,5R)-5-amino-1,1-dioxidotetrahydro-2H-thiopyran-3-yl)-4-fluorobenzamide (40 mg, 0.14 mmol) and 4,6-dichloro-2-(trifluoromethyl)quinoline (41 mg, 0.15 mmol) were dissolved in DMSO (1.4 mL). The reaction was heated to 90° C. for 12 h. The reaction was cooled to room temperature and water was added. The aqueous layer was washed with EtOAc and the combined organic layer was dried over MgSO₄. The reaction mixture was purified by reverse phase HPLC (20-95% MeCN/H2O) to give the title compound (1 mg, 2%). ESI-MS m/z: 516.08 [M+H].

The following examples are prepared using procedures similar to those described above:

| Example | Structure |
|---|---|
| 1a | 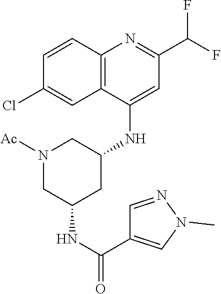 [SMILES: CC(=O)N1C[C@@H](NC(=O)c2cnn(C)c2)C[C@@H](Nc2cc(C(F)F)nc3ccc(Cl)cc23)C1] |
| 2a | 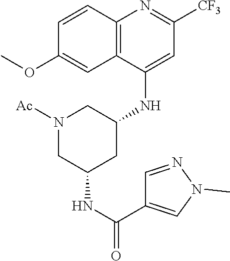 |
| 3a | 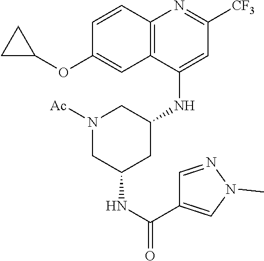 [SMILES: CC(=O)N1C[C@@H](NC(=O)c2cnn(C)c2)C[C@@H](Nc2cc(C(F)(F)F)nc3ccc(OC4CC4)cc23)C1] |
| 4a | 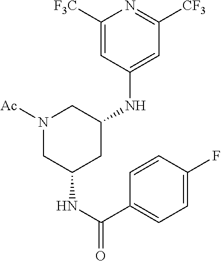 [SMILES: CC(=O)N1C[C@@H](NC(=O)c2ccc(F)cc2)C[C@@H](Nc2cc(C(F)(F)F)nc(C(F)(F)F)c2)C1] |
| 5a | 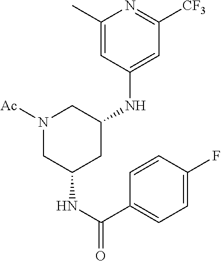 [SMILES: CC(=O)N1C[C@@H](NC(=O)c2ccc(F)cc2)C[C@@H](Nc2cc(C)nc(C(F)(F)F)c2)C1] |
| 6a | 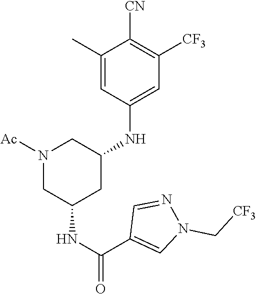 [SMILES: CC(=O)N1C[C@@H](NC(=O)c2cnn(CC(F)(F)F)c2)C[C@@H](Nc2cc(C)c(C#N)c(C(F)(F)F)c2)C1] |
| 7a | 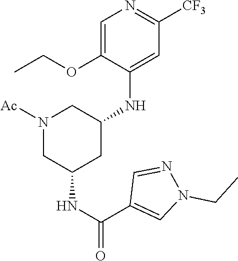 [SMILES: CCOc1cnc(C(F)(F)F)cc1N[C@@H]1C[C@H](NC(=O)c2cnn(CC)c2)CN(C(C)=O)C1] |
| 8a | 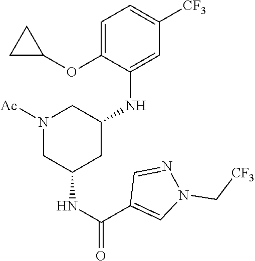 [SMILES: CC(=O)N1C[C@@H](NC(=O)c2cnn(CC(F)(F)F)c2)C[C@@H](Nc2cc(C(F)(F)F)ccc2OC2CC2)C1] |
| 9a | 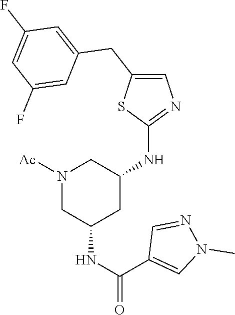 [SMILES: CC(=O)N1C[C@@H](NC(=O)c2cnn(C)c2)C[C@@H](Nc2ncc(Cc3cc(F)cc(F)c3)s2)C1] |
| 10a | 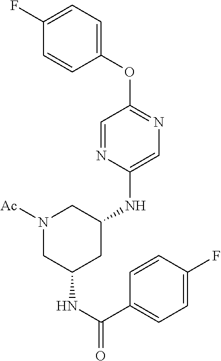 [SMILES: CC(=O)N1C[C@@H](NC(=O)c2ccc(F)cc2)C[C@@H](Nc2cnc(Oc3ccc(F)cc3)cn2)C1] |
| 11a | 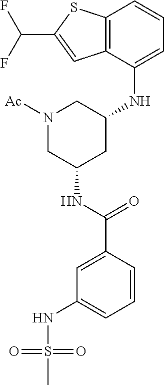 [SMILES: CC(=O)N1C[C@@H](NC(=O)c2cccc(NS(C)(=O)=O)c2)C[C@@H](Nc2cccc3sc(C(F)F)cc23)C1] |
| 12a | 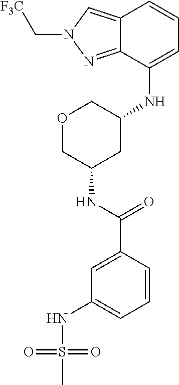 [SMILES: CS(=O)(=O)Nc1cccc(C(=O)N[C@@H]2COC[C@H](Nc3cccc4cn(CC(F)(F)F)nc34)C2)c1] |
| 13a | 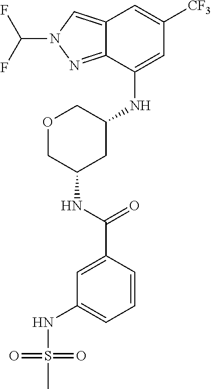 [SMILES: CS(=O)(=O)Nc1cccc(C(=O)N[C@@H]2COC[C@H](Nc3cc(C(F)(F)F)cc4cn(C(F)F)nc34)C2)c1] |
| 14a | 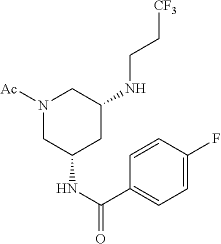 [SMILES: CC(=O)N1C[C@H](NCCC(F)(F)F)C[C@H](NC(=O)c2ccc(F)cc2)C1] |
| 15a | 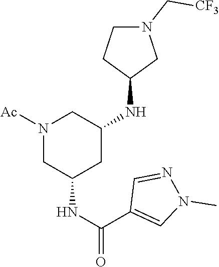 [SMILES: CC(=O)N1C[C@@H](NC(=O)c2cnn(C)c2)C[C@@H](N[C@H]2CCN(CC(F)(F)F)C2)C1] |
| 16a | 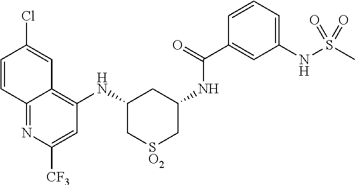 [SMILES: CS(=O)(=O)Nc1cccc(C(=O)N[C@H]2C[C@@H](Nc3cc(C(F)(F)F)nc4ccc(Cl)cc34)CS(=O)(=O)C2)c1] |
| 17a | 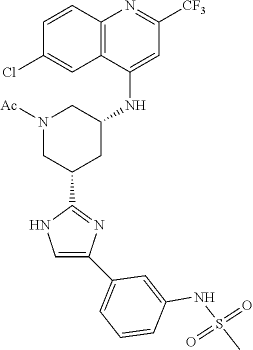 [SMILES: CC(=O)N1C[C@H](Nc2cc(C(F)(F)F)nc3ccc(Cl)cc23)C[C@H](c2nc(-c3cccc(NS(C)(=O)=O)c3)c[nH]2)C1] |
| 18a | 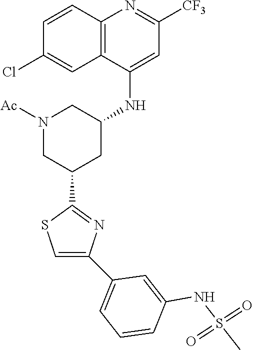 [SMILES: CC(=O)N1C[C@H](Nc2cc(C(F)(F)F)nc3ccc(Cl)cc23)C[C@H](c2nc(-c3cccc(NS(C)(=O)=O)c3)cs2)C1] |
| 19a | 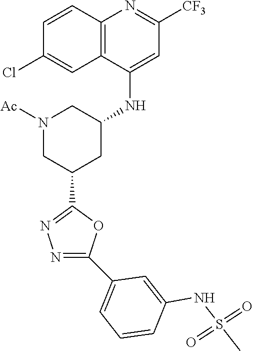 [SMILES: CC(=O)N1C[C@H](Nc2cc(C(F)(F)F)nc3ccc(Cl)cc23)C[C@H](c2nnc(-c3cccc(NS(C)(=O)=O)c3)o2)C1] |
| 20a | 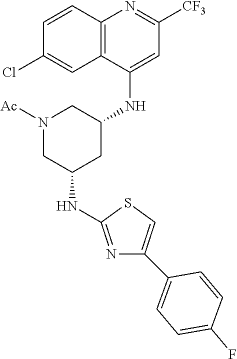 [SMILES: CC(=O)N1C[C@@H](Nc2nc(-c3ccc(F)cc3)cs2)C[C@@H](Nc2cc(C(F)(F)F)nc3ccc(Cl)cc23)C1] |
| 21a | 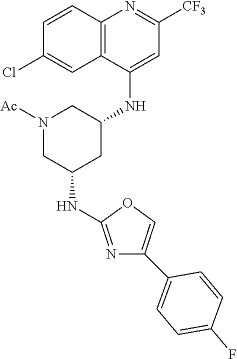 [SMILES: CC(=O)N1C[C@@H](Nc2nc(-c3ccc(F)cc3)co2)C[C@@H](Nc2cc(C(F)(F)F)nc3ccc(Cl)cc23)C1] |
| 22a | 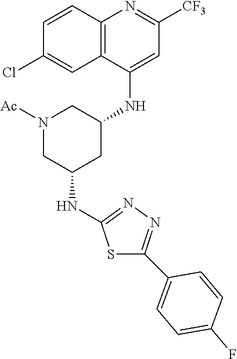 [SMILES: CC(=O)N1C[C@@H](Nc2nnc(-c3ccc(F)cc3)s2)C[C@@H](Nc2cc(C(F)(F)F)nc3ccc(Cl)cc23)C1] |
| 23a | 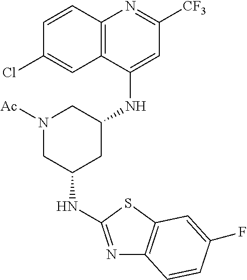 [SMILES: CC(=O)N1C[C@@H](Nc2nc3ccc(F)cc3s2)C[C@@H](Nc2cc(C(F)(F)F)nc3ccc(Cl)cc23)C1] |
| 24a | 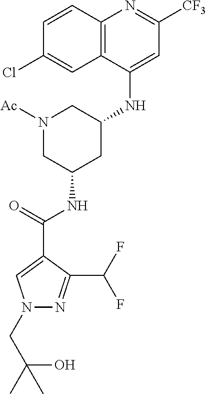 [SMILES: CC(=O)N1C[C@@H](NC(=O)c2cn(CC(C)(C)O)nc2C(F)F)C[C@@H](Nc2cc(C(F)(F)F)nc3ccc(Cl)cc23)C1] |
| 25a | 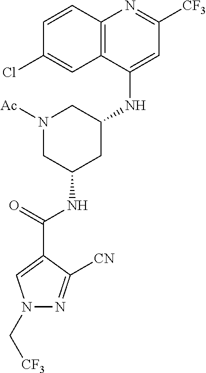 [SMILES: CC(=O)N1C[C@@H](NC(=O)c2cn(CC(F)(F)F)nc2C#N)C[C@@H](Nc2cc(C(F)(F)F)nc3ccc(Cl)cc23)C1] |
| 26a | 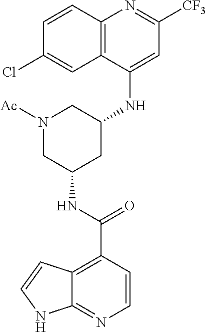 [SMILES: CC(=O)N1C[C@@H](NC(=O)c2ccnc3[nH]ccc23)C[C@@H](Nc2cc(C(F)(F)F)nc3ccc(Cl)cc23)C1] |
| 27a | 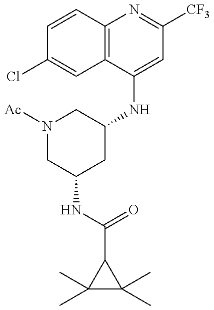 [SMILES: CC(=O)N1C[C@@H](NC(=O)C2C(C)(C)C2(C)C)C[C@@H](Nc2cc(C(F)(F)F)nc3ccc(Cl)cc23)C1] |
| 28a | 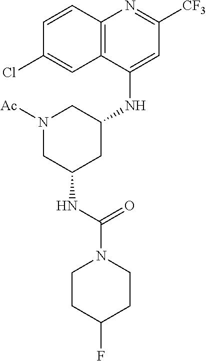 [SMILES: CC(=O)N1C[C@@H](NC(=O)N2CCC(F)CC2)C[C@@H](Nc2cc(C(F)(F)F)nc3ccc(Cl)cc23)C1] |
| 29a | 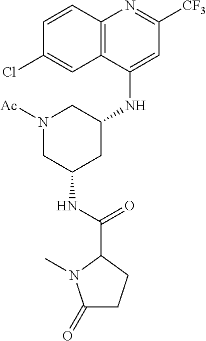 [SMILES: CC(=O)N1C[C@@H](NC(=O)C2CCC(=O)N2C)C[C@@H](Nc2cc(C(F)(F)F)nc3ccc(Cl)cc23)C1] |
| 30a | 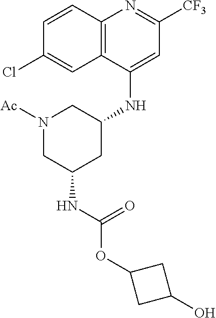 [SMILES: CC(=O)N1C[C@@H](NC(=O)OC2CC(O)C2)C[C@@H](Nc2cc(C(F)(F)F)nc3ccc(Cl)cc23)C1] |
| 31a | 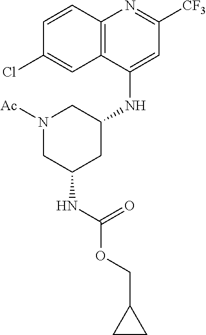 [SMILES: CC(=O)N1C[C@@H](NC(=O)OCC2CC2)C[C@@H](Nc2cc(C(F)(F)F)nc3ccc(Cl)cc23)C1] |
| 32a | 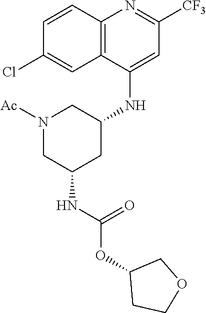 [SMILES: CC(=O)N1C[C@@H](NC(=O)O[C@H]2CCOC2)C[C@@H](Nc2cc(C(F)(F)F)nc3ccc(Cl)cc23)C1] |
| 33a | 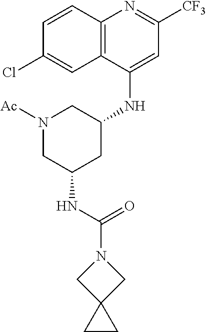 [SMILES: CC(=O)N1C[C@@H](NC(=O)N2CC3(CC3)C2)C[C@@H](Nc2cc(C(F)(F)F)nc3ccc(Cl)cc23)C1] |
| 34a | 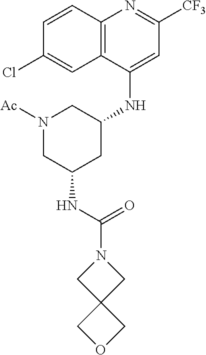 [SMILES: CC(=O)N1C[C@@H](NC(=O)N2CC3(COC3)C2)C[C@@H](Nc2cc(C(F)(F)F)nc3ccc(Cl)cc23)C1] |
| 35a | 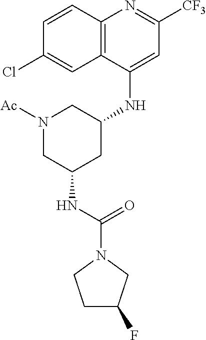 [SMILES: CC(=O)N1C[C@@H](NC(=O)N2CC[C@H](F)C2)C[C@@H](Nc2cc(C(F)(F)F)nc3ccc(Cl)cc23)C1] |
| 36a | 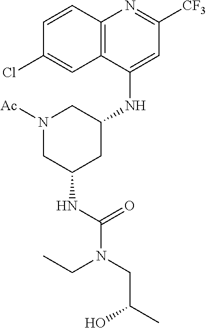 [SMILES: CCN(C[C@H](C)O)C(=O)N[C@H]1C[C@@H](Nc2cc(C(F)(F)F)nc3ccc(Cl)cc23)CN(C(C)=O)C1] |
| 37a | 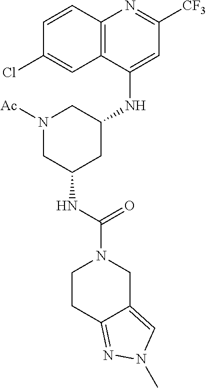 [SMILES: CC(=O)N1C[C@@H](NC(=O)N2CCc3nn(C)cc3C2)C[C@@H](Nc2cc(C(F)(F)F)nc3ccc(Cl)cc23)C1] |
| 38a | 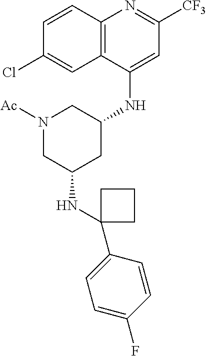 [SMILES: CC(=O)N1C[C@H](Nc2cc(C(F)(F)F)nc3ccc(Cl)cc23)C[C@H](NC2(c3ccc(F)cc3)CCC2)C1] |
| 39a | 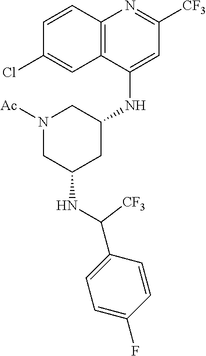 [SMILES: CC(=O)N1C[C@H](Nc2cc(C(F)(F)F)nc3ccc(Cl)cc23)C[C@H](NC(c2ccc(F)cc2)C(F)(F)F)C1] |

### Biological Activity

### MRGPRX2 Activity

HEK293 cells stably expressing human MRGPRX2 were grown in Dulbecco's Modified Eagle Medium (DMEM) supplemented with 10% fetal bovine serum (FBS), 1% Glutamax, and 1 μg/mL of puromycin. Cells were maintained in a 37° C., 5% CO₂ humidified incubator.

Cells were plated in a 384-well assay plate at a density of 20,000 cells per well overnight. Compounds were solubilized at 10 mM in DMSO and added as a 10-point curve using an ECHO acoustic liquid handler. On the day of the assay, Cortistatin-14 was diluted in assay buffer (provided by IP-One-Gq-HTRF kit purchased from Cisbio, 62IPAPEJ) and added to the assay plate at a final concentration corresponding to the predetermined EC75. Plates were incubated at 37° C. for 2 hours prior to addition of HTRF detection reagents per the manufacturer's instructions. The plates were read on a Perkin Elmer Envision plate reader. The HTRF ratio and cellular IP-1 levels were calculated. XLfit was used to calculate an EC50 value for each compound using a 4-parameter logistic curve fitting model. EC₅₀ ranges are reported as follows: A<100 nM; B 0.1-1 μM; C>1 μM.

TABLE 1
Summary of Biological Activities

| | MRGPRX2 IP-1 Gq | | MRGPRX2 IP-1 Gq |
|---|---|---|---|
| Example # | HTRF EC50 (nM) | Example # | HTRF EC50 (nM) |
| 1 | B | 2 | A |
| 3 | B | 4 | B |
| 5 | A | 6 | B |
| 7 | C | 8 | B |
| 9 | B | 10 | B |
| 11 | A | 12 | B |
| 13 | B | 14 | A |
| 15 | B | 16 | B |
| 17 | B | 18 | B |
| 19 | B | 20 | B |
| 21 | A | 22 | B |
| 23 | A | 24 | A |
| 25 | A | 26 | B |
| 27 | B | 28 | A |
| 29 | A | 30 | A |
| 31 | A | 32 | B |
| 33 | B | 34 | C |
| 35 | B | 36 | A |
| 37 | B | 38 | B |
| 39 | B | 40 | A |
| 41 | B | 42 | A |
| 43 | B | 44 | A |
| 45 | C | 46 | C |
| 47 | C | 48 | C |
| 49 | C | 50 | C |
| 51 | C | 52 | C |
| 53 | A | 54 | B |
| 55 | B | 56 | A |
| 57 | A | 58 | A |
| 59 | A | 60 | B |
| 61 | A | 62 | B |
| 63 | B | 64 | A |
| 65 | A | 66 | B |
| 67 | B | 68 | A |
| 69 | B | 70 | B |
| 71 | C | 73 | A |
| 140 | C. | | |

LAD2 β-hexosaminidase Release Assay

LAD2 cells obtained from the NIH were cultured in StemPro34 (Gibco) serum-free media supplemented with GlutaMax, penicillin and streptomycin, and 100 ng/mL SCF (Invitrogen). Cells were maintained in a 37° C., 5% CO₂ humidified incubator at a concentration of 2-5E5 cells/mL, with hemidepletion once a week.

One day prior to the assay, cells were starved of SCF at a concentration of 2.3E5 cells/mL. On the day of the assay cells were washed twice in Tyrode's Buffer containing 0.04% BSA (assay buffer) and plated in a 96 well-v-bottom assay plate at 20,000 cells per well in 80 μL of assay buffer. Compounds were solubilized at 10 mM DMSO and added as a 10-fold overconcentrated 8-point curve in assay buffer in a volume of 10 μL using the JANUS liquid handler. Assay plates were incubated for 1 hour at 37° C. Cortistatin-14 was diluted in assay buffer to 10λ its predetermined EC₇₅ and added to the assay plates in a volume of 10 μL. Assay plates were incubated at 37° C. for 30 minutes. After incubation, cells were pelleted and 50 μL of supernatant from each well was transferred to a 96 well flat bottom plate containing 100 μL of substrate solution (3.5 mg/mL p-Nitrophenyl-N-acetyl-β-D-glucosaminide (Sigma Aldrich) in 40 mM Citrate Buffer (Thermo Fisher)). The remaining cell pellets were lysed with 150 μL of 0.1% Triton-X-100 and mixed thoroughly. 50 μL of lysate was transferred to a 96-well flat bottom plate containing 100 μL of substrate solution. Plates with supernatants and lysates were incubated at 37° C. for 90 minutes prior to addition of 50 μL of 400 mM Glycine buffer (pH 10.7). The plates were read on a Perkin Elmer Envision plate reader at an Abs of 405 nanometers. Percent degranulation was calculated and Xlfit was used to calculate an EC₅₀ value for each compound using a 4-parameter logistic curve fitting model. EC50 ranges are reported as follows: A<100 nM; B 0.1-1 μM; C>1 μM.

TABLE 1
Summary of Biological Activities

| | MRGPRX2 LAD2 | | MRGPRX2 LAD2 |
|---|---|---|---|
| Example # | EC50 (nM) | Example # | EC50 (nM) |
| 1 | A | 2 | A |
| 3 | A | 4 | A |
| 5 | A | 8 | A |
| 9 | B | 10 | A |
| 24 | A | 25 | A |
| 29 | A | 30 | A |
| 32 | B | 41 | A |
| 42 | A | 43 | A |
| 44 | A | 72 | A |
| 74 | A | 75 | A |
| 76 | B | 77 | A |
| 78 | A | 79 | A |
| 80 | A | 81 | A |
| 82 | A | 83 | A |
| 84 | A | 85 | A |
| 86 | A | 87 | A |
| 88 | A | 89 | A |
| 90 | A | 91 | B |
| 92 | A | 93 | A |
| 94 | A | 95 | A |
| 96 | A | 97 | A |
| 98 | A | 99 | A |
| 100 | A | 101 | A |
| 102 | A | 103 | A |
| 104 | A | 105 | A |
| 106 | A | 108 | A |
| 109 | A | 110 | A |
| 111 | A | 112 | A |
| 113 | A | 114 | A |
| 115 | A | 116 | A |
| 117 | A | 118 | A |
| 119 | A | 120 | A |
| 121 | A | 122 | A |
| 123 | A | 124 | A |
| 125 | A | 126 | A |
| 127 | A | 128 | A |
| 129 | A | 130 | A |
| 132 | A | 133 | A |
| 135 | A | 136 | A |
| 139 | A. | | |

It is understood that the examples and embodiments described herein are for illustrative purposes only and that various modifications or changes in light thereof will be suggested to persons skilled in the art and are to be included within the spirit and purview of this application and scope of the appended claims.

While this invention has been particularly shown and described with references to preferred embodiments thereof, it will be understood by those skilled in the art that various changes in form and details may be made therein without departing from the scope of the invention encompassed by the appended claims.

1. A compound of Formula (I) or pharmaceutically acceptable salt thereof: 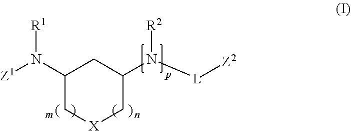
wherein:
Z¹ is selected from the group consisting of optionally substituted aryl, optionally substituted heteroaryl, optionally substituted —C₁-C₈ alkyl, optionally substituted —C₁-C₈ alkoxy, optionally substituted —C₃-C₁₂ cycloalkyl, and optionally substituted 3- to 12-membered heterocycloalkyl;
R¹ is selected from the group consisting of hydrogen, optionally substituted —C₁-C₆ alkyl, optionally substituted —C₃-C₈ cycloalkyl, optionally substituted 3- to 8-membered heterocycloalkyl, —C(O)R¹¹, —C(O)OR¹¹, —C(O)N(R¹²)(R¹³) and —S(O)₂R¹¹;
R¹¹ is selected from the group consisting of: optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted aryl, optionally substituted arylalkyl, optionally substituted heteroarylalkyl, and optionally substituted heteroaryl;
R¹² and R¹³ are each independently selected from the group consisting of: hydrogen, optionally substituted —C₁-C₈ alkyl, optionally substituted —C₂-C₈ alkenyl, optionally substituted —C₃-C₈ cycloalkyl, optionally substituted 3- to 8-membered heterocycloalkyl, optionally substituted aryl, optionally substituted arylalkyl, optionally substituted heteroarylalkyl, and optionally substituted heteroaryl; alternatively, R¹² and R¹³ are taken together with the nitrogen atom to which they are attached to form an optionally substituted 3-8 membered heterocyclic containing 0, 1, 2, or 3 double bonds;
alternatively, R¹ and Z¹ are taken together with the nitrogen atom to which they are attached to form an optionally substituted heterocyclic or an optionally substituted heteroaryl;
R² is selected from the group consisting of hydrogen, optionally substituted —C₁-C₆ alkyl, and optionally substituted —C₃-C₈ cycloalkyl;
p is 0 or 1;
L is-C(O)—, —S(O)₂—, or absent;
Z² is selected from the group consisting of:
1) Optionally substituted aryl;
2) Optionally substituted heteroaryl;
3) Optionally substituted —C₃-C₁₂ cycloalkyl;
4) Optionally substituted —C₃-C₁₂ cycloalkenyl;
5) Optionally substituted 3- to 12-membered heterocycloalkyl;
6) Optionally substituted —C₁-C₈ alkyl;
7) Optionally substituted arylalkyl;
8) Optionally substituted heteroarylalkyl;
9) —N(R¹²)(R¹³); and
10) —OR¹¹;
m is 1 or 2;
n is 1 or 2; and
X is —NR³—, —O—, —S—, —S(O)—, or —S(O)₂—;
R³ is selected from the group consisting of:
1) Hydrogen;
2) Optionally substituted —C₁-C₈ alkyl;
3) Optionally substituted —C₃-C₈ cycloalkyl;
4) Optionally substituted 3- to 8-membered heterocycloalkyl;
5) Optionally substituted aryl;
6) Optionally substituted heteroaryl;
7) Optionally substituted arylalkyl;
8) Optionally substituted heteroarylalkyl;
9) C(O)F
10) —C(O)R¹¹;
11) —C(O)OR¹¹;
12) —C(O)N(R¹²)(R¹³); and
13) —S(O)₂R¹¹.

2. The compound of claim 1 represented by Formula (II): 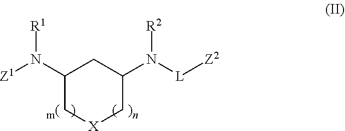
wherein R¹, R², Z¹, Z², X, L, m and n are as defined in claim 1.

3. The compound of claim 1 represented by one of Formulae (XI-1)˜(XI-9): 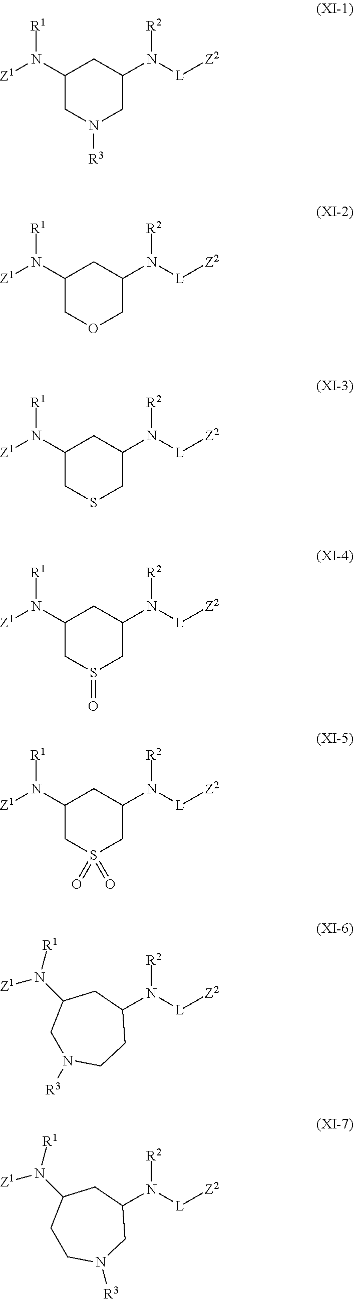 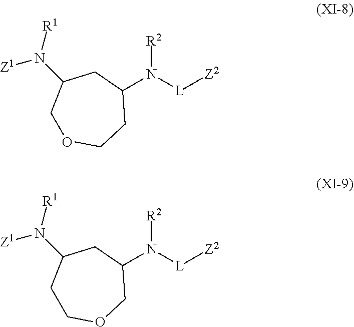
wherein R¹, R², R³, Z¹, Z², and L are as defined in claim 1.

4. The compound of claim 1 represented by one of Formulae (XII-1)˜(XII-9): 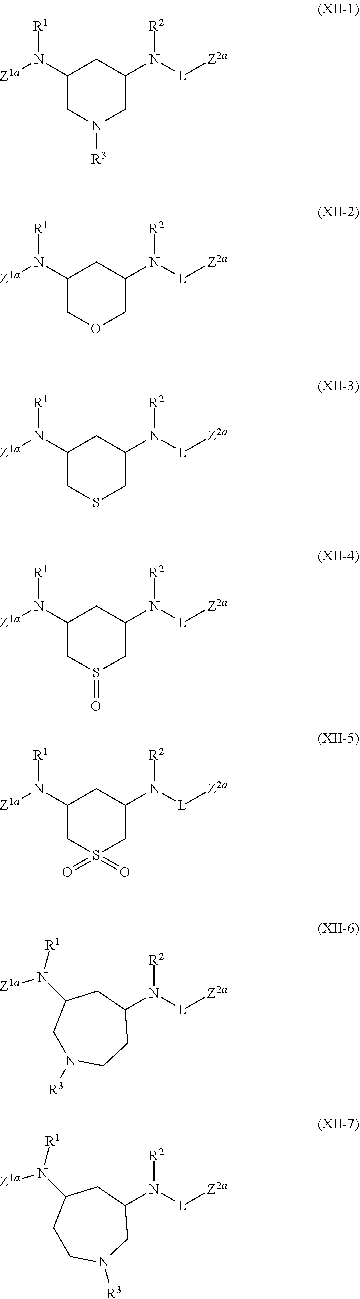 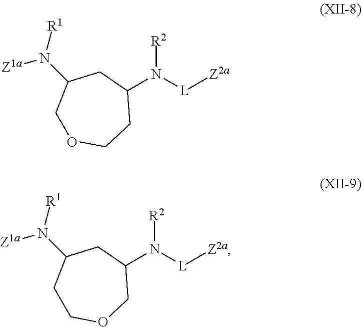
wherein Z^{1a} is optionally substituted —C₃-C₁₂ cycloalkyl, optionally substituted 3- to 12-membered heterocycloalkyl, optionally substituted aryl, or optionally substituted heteroaryl; Z^{2a} is optionally substituted —C₃-C₁₂ cycloalkyl, optionally substituted 3- to 12-membered heterocycloalkyl, optionally substituted aryl, or optionally substituted heteroaryl; and R¹, R², and R³ are as defined in claim 1.

5. The compound of claim 1 represented by one of Formulae (XV-1)˜(XV-3): 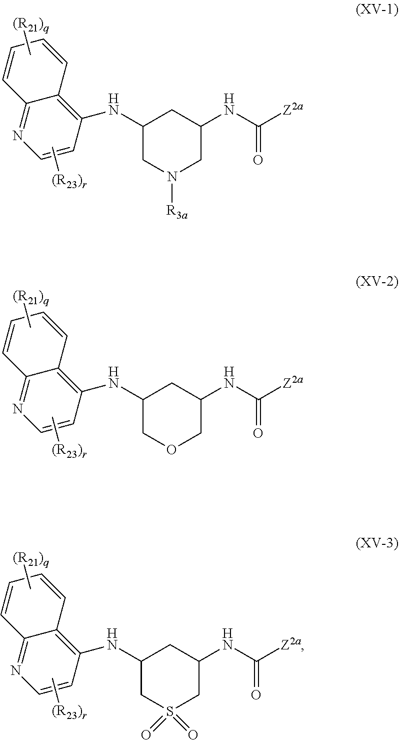
wherein
q is 0, 1, 2, 3, or 4; r is 0, 1 or 2;
each R₂₁ and R₂₃ is independently selected from the group consisting of:
1) halogen;
2) —CN;
3) —OH;
4) —OR¹¹;
5) —NR¹²R¹³;
6) Optionally substituted —C₁-C₈ alkyl;
7) Optionally substituted —C₁-C₈ haloalkyl;
8) Optionally substituted —C₃-C₈ cycloalkyl;
9) Optionally substituted 3- to 8-membered heterocycloalkyl;
10) Optionally substituted aryl; and
11) Optionally substituted heteroaryl;
R₃ₐ is selected from the group consisting of:
1) —C(O)R¹¹;
2) —C(O)₂R¹¹;
3) —SO₂R¹¹; and
4) Optionally substituted —C₁-C₈alkyl;
Z^{2a} is optionally substituted —C₃-C₁₂ cycloalkyl, optionally substituted 3- to 12-membered heterocycloalkyl, optionally substituted aryl or optionally substituted heteroaryl; and R¹¹, R¹², and R¹³ are as defined in claim 1.

6. The compound of claim 1 represented by one of Formulae (XV-1a)˜(XV-3a): 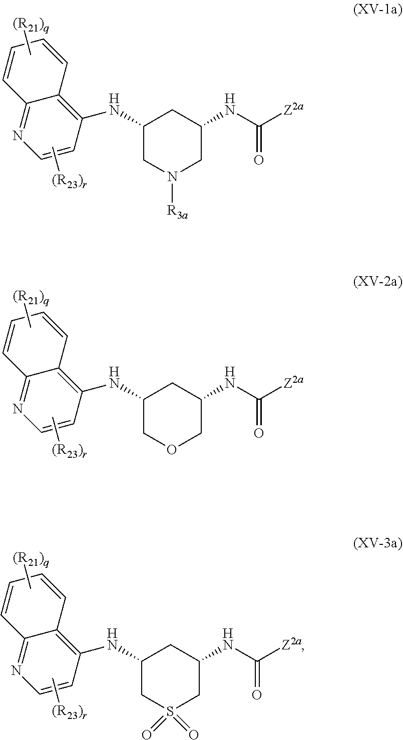
wherein
q is 0, 1, 2, 3, or 4; r is 0, 1 or 2;
each R₂₁ and R₂₃ is independently selected from the group consisting of:
1) halogen;
2) —CN;
3) —OH;
4) —OR¹¹;
5) —NR¹²R¹³;
6) Optionally substituted —C₁-C₈ alkyl;
7) Optionally substituted —C₁-C₈ haloalkyl;
8) Optionally substituted —C₃-C₈ cycloalkyl;
9) Optionally substituted 3- to 8-membered heterocycloalkyl;
10) Optionally substituted aryl; and
11) Optionally substituted heteroaryl;
R₃ₐ is selected from the group consisting of:
1) Hydrogen;
2) —C(O)R¹¹;
3) —C(O)₂R¹¹;
4) —SO₂R¹¹;
5) Optionally substituted —C₁-C₈ alkyl;
6) Optionally substituted 3- to 8-membered heterocycloalkyl;
7) Optionally substituted aryl; and
8) Optionally substituted heteroaryl;
Z^{2a} is optionally substituted —C₃-C₁₂ cycloalkyl, optionally substituted 3- to 12-membered heterocycloalkyl, optionally substituted aryl or optionally substituted heteroaryl; and R¹¹, R¹², and R¹³ are as defined in claim 1.

7. The compound of claim 1 represented by one of Formulae (XV-4)˜(XV-6): 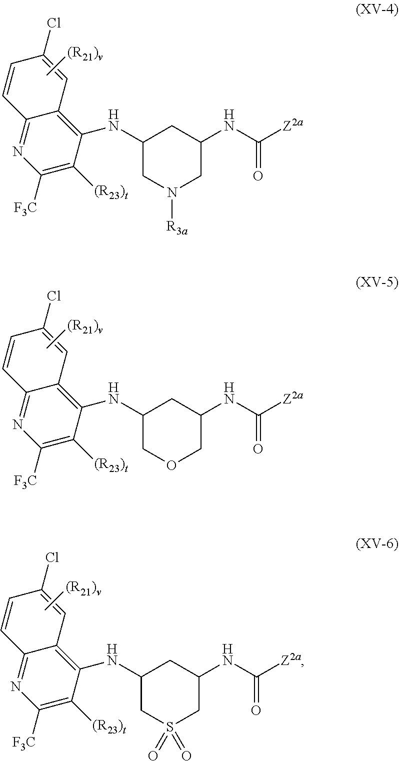
wherein
v is 0 or 1; t is 0 or 1;
each R₂₁ and R₂₃ is independently selected from the group consisting of:
1) halogen;
2) —CN;
3) —OH;
4) —OR¹¹;
5) —NR¹²R¹³;
6) Optionally substituted —C₁-C₈alkyl;
7) Optionally substituted —C₁-C₈ haloalkyl;
8) Optionally substituted —C₃-C₈ cycloalkyl;
9) Optionally substituted 3- to 8-membered heterocycloalkyl;
10) Optionally substituted aryl; and
11) Optionally substituted heteroaryl;
R₃ₐ is selected from the group consisting of:
1) Hydrogen;
2) —C(O)R¹¹;
3) —C(O)₂R¹¹;
4) —SO₂R¹¹;
5) Optionally substituted —C₁-C₈alkyl;
6) Optionally substituted 3- to 8-membered heterocycloalkyl;
7) Optionally substituted aryl; and
8) Optionally substituted heteroaryl;
Z^{2a} is optionally substituted —C₃-C₁₂ cycloalkyl, optionally substituted 3- to 12-membered heterocycloalkyl, optionally substituted aryl or optionally substituted heteroaryl; and R¹¹, R¹², and R¹³ are as defined in claim 1.

8. The compound of claim 1 represented by one of Formulae (XVII-1a)˜(XVII— 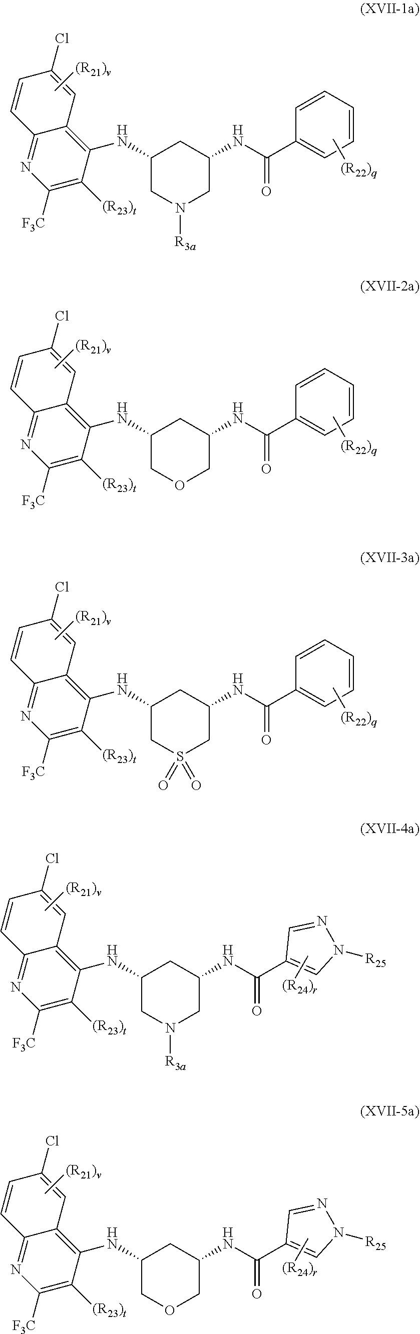 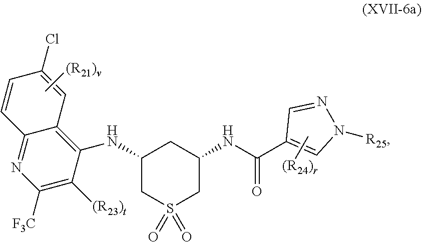
wherein:
v is 0 or 1; t is 0 or 1; q is 0, 1, 2, 3 or 4; r is 0, 1 or 2;
each R₂₁ and R₂₃ is independently selected from the group consisting of:
1) halogen;
2) —CN;
3) —OH;
4) —OR¹¹;
5) —NR¹²R¹³;
6) Optionally substituted —C₁-C₈ alkyl;
7) Optionally substituted —C₁-C₈ haloalkyl;
8) Optionally substituted —C₃-C₈ cycloalkyl;
9) Optionally substituted 3- to 8-membered heterocycloalkyl;
10) Optionally substituted aryl; and
11) Optionally substituted heteroaryl;
each R₂₂ is independently selected from the group consisting of:
1) halogen;
2) —CN;
3) —OH;
4) —OR¹¹;
5) —NR¹²R¹³;
6) Optionally substituted —C₁-C₈alkyl;
7) Optionally substituted —C₁-C₈ haloalkyl;
8) Optionally substituted —C₃-C₈ cycloalkyl;
9) Optionally substituted 3- to 8-membered heterocycloalkyl;
10) Optionally substituted aryl; and
11) Optionally substituted heteroaryl;
each R₂₄ is independently selected from the group consisting of:
1) halogen;
2) —NR¹²R¹³;
3) Optionally substituted —C₁-C₈ alkyl;
4) Optionally substituted —C₁-C₈ haloalkyl;
5) Optionally substituted —C₃-C₈ cycloalkyl;
6) Optionally substituted 3- to 8-membered heterocycloalkyl;
7) Optionally substituted aryl; and
8) Optionally substituted heteroaryl;
each R₂₅ is independently selected from the group consisting of:
1) hydrogen;
2) Optionally substituted —C₁-C₈ alkyl;
3) Optionally substituted —C₁-C₈ haloalkyl;
4) Optionally substituted —C₃-C₈ cycloalkyl;
5) Optionally substituted 3- to 8-membered heterocycloalkyl;
6) Optionally substituted aryl; and
7) Optionally substituted heteroaryl;
and R₃ₐ is selected from the group consisting of:
1) Hydrogen;
2) —C(O)R¹¹;
3) —C(O)₂R¹¹;
4) —SO₂R¹¹;
5) Optionally substituted —C₁-C₈ alkyl;
6) Optionally substituted 3- to 8-membered heterocycloalkyl;
7) Optionally substituted aryl; and
8) Optionally substituted heteroaryl.

9. A compound selected from the compounds set forth below, or a pharmaceutically acceptable salt thereof:

| Compound | Structure |
|---|---|
| 1 | 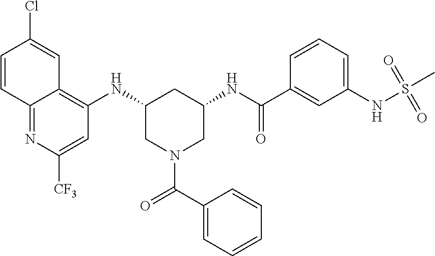 [SMILES: CS(=O)(=O)Nc1cccc(C(=O)N[C@H]2C[C@@H](Nc3cc(C(F)(F)F)nc4ccc(Cl)cc34)CN(C(=O)c3ccccc3)C2)c1] |
| 2 | 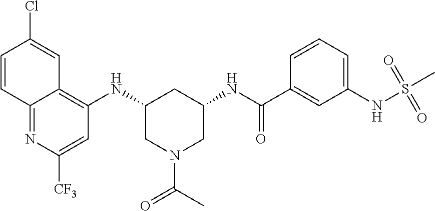 [SMILES: CC(=O)N1C[C@@H](NC(=O)c2cccc(NS(C)(=O)=O)c2)C[C@@H](Nc2cc(C(F)(F)F)nc3ccc(Cl)cc23)C1] |
| 3 | 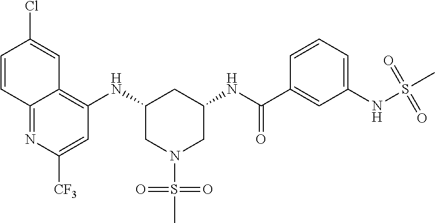 [SMILES: CS(=O)(=O)Nc1cccc(C(=O)N[C@H]2C[C@@H](Nc3cc(C(F)(F)F)nc4ccc(Cl)cc34)CN(S(C)(=O)=O)C2)c1] |
| 4 | 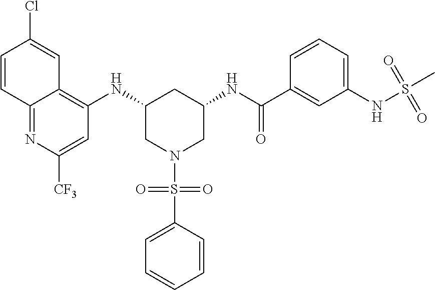 [SMILES: CS(=O)(=O)Nc1cccc(C(=O)N[C@H]2C[C@@H](Nc3cc(C(F)(F)F)nc4ccc(Cl)cc34)CN(S(=O)(=O)c3ccccc3)C2)c1] |
| 5 | 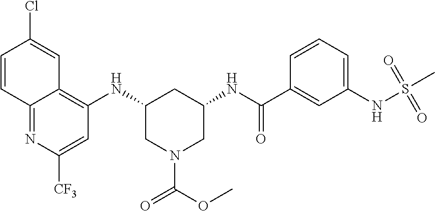 [SMILES: COC(=O)N1C[C@@H](NC(=O)c2cccc(NS(C)(=O)=O)c2)C[C@@H](Nc2cc(C(F)(F)F)nc3ccc(Cl)cc23)C1] |
| 6 | 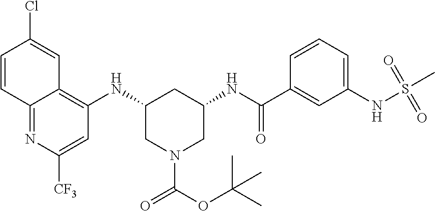 [SMILES: CC(C)(C)OC(=O)N1C[C@@H](NC(=O)c2cccc(NS(C)(=O)=O)c2)C[C@@H](Nc2cc(C(F)(F)F)nc3ccc(Cl)cc23)C1] |
| 7 | 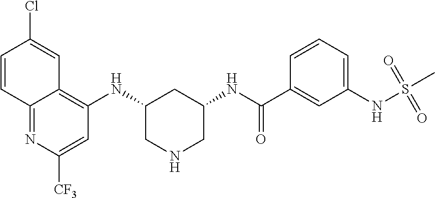 [SMILES: CS(=O)(=O)Nc1cccc(C(=O)N[C@@H]2CNC[C@H](Nc3cc(C(F)(F)F)nc4ccc(Cl)cc34)C2)c1] |
| 8 | 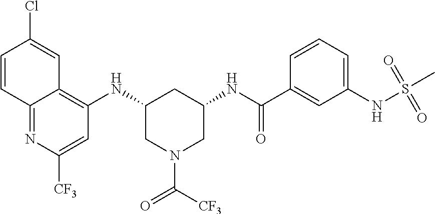 [SMILES: CS(=O)(=O)Nc1cccc(C(=O)N[C@H]2C[C@@H](Nc3cc(C(F)(F)F)nc4ccc(Cl)cc34)CN(C(=O)C(F)(F)F)C2)c1] |
| 9 | 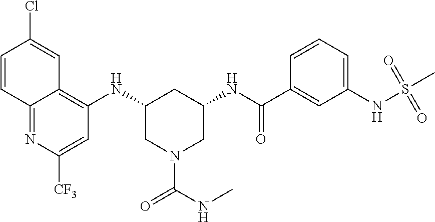 [SMILES: CNC(=O)N1C[C@@H](NC(=O)c2cccc(NS(C)(=O)=O)c2)C[C@@H](Nc2cc(C(F)(F)F)nc3ccc(Cl)cc23)C1] |
| 10 | 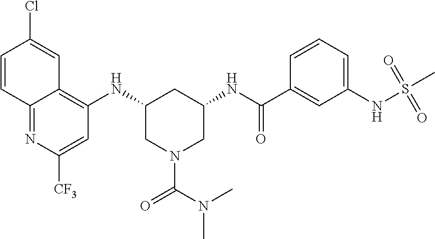 [SMILES: CN(C)C(=O)N1C[C@@H](NC(=O)c2cccc(NS(C)(=O)=O)c2)C[C@@H](Nc2cc(C(F)(F)F)nc3ccc(Cl)cc23)C1] |
| 11 | 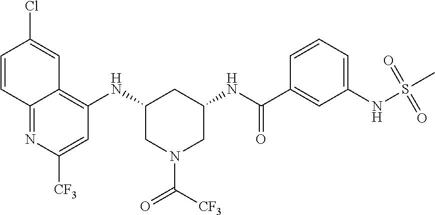 [SMILES: CS(=O)(=O)Nc1cccc(C(=O)N[C@H]2C[C@@H](Nc3cc(C(F)(F)F)nc4ccc(Cl)cc34)CN(C(=O)C(F)(F)F)C2)c1] |
| 12 | 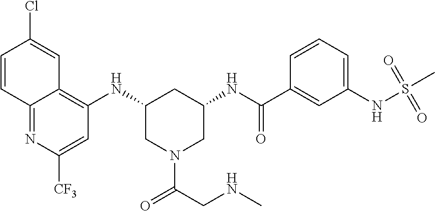 [SMILES: CNCC(=O)N1C[C@@H](NC(=O)c2cccc(NS(C)(=O)=O)c2)C[C@@H](Nc2cc(C(F)(F)F)nc3ccc(Cl)cc23)C1] |
| 13 | 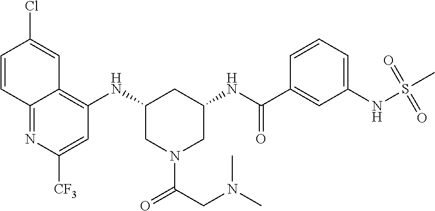 [SMILES: CN(C)CC(=O)N1C[C@@H](NC(=O)c2cccc(NS(C)(=O)=O)c2)C[C@@H](Nc2cc(C(F)(F)F)nc3ccc(Cl)cc23)C1] |
| 14 | 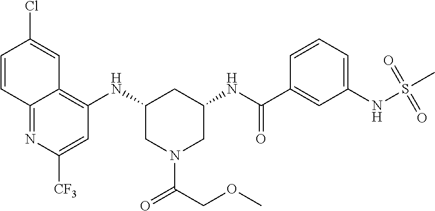 [SMILES: COCC(=O)N1C[C@@H](NC(=O)c2cccc(NS(C)(=O)=O)c2)C[C@@H](Nc2cc(C(F)(F)F)nc3ccc(Cl)cc23)C1] |
| 15 | 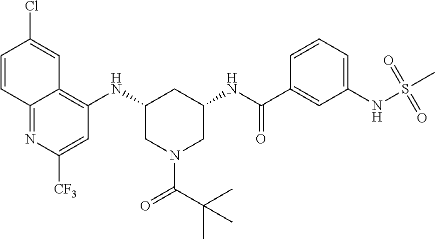 [SMILES: CC(C)(C)C(=O)N1C[C@@H](NC(=O)c2cccc(NS(C)(=O)=O)c2)C[C@@H](Nc2cc(C(F)(F)F)nc3ccc(Cl)cc23)C1] |
| 16 | 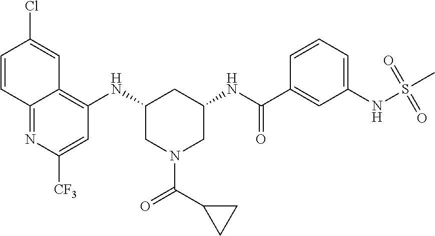 [SMILES: CS(=O)(=O)Nc1cccc(C(=O)N[C@H]2C[C@@H](Nc3cc(C(F)(F)F)nc4ccc(Cl)cc34)CN(C(=O)C3CC3)C2)c1] |
| 17 | 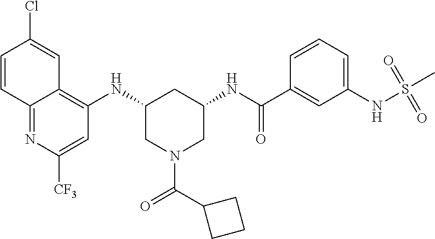 [SMILES: CS(=O)(=O)Nc1cccc(C(=O)N[C@H]2C[C@@H](Nc3cc(C(F)(F)F)nc4ccc(Cl)cc34)CN(C(=O)C3CCC3)C2)c1] |
| 18 | 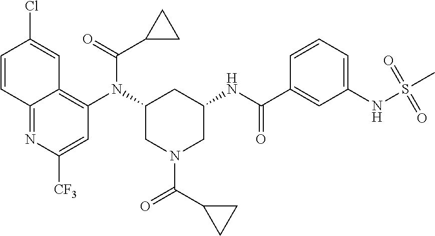 [SMILES: CS(=O)(=O)Nc1cccc(C(=O)N[C@H]2C[C@@H](N(C(=O)C3CC3)c3cc(C(F)(F)F)nc4ccc(Cl)cc34)CN(C(=O)C3CC3)C2)c1] |
| 19 | 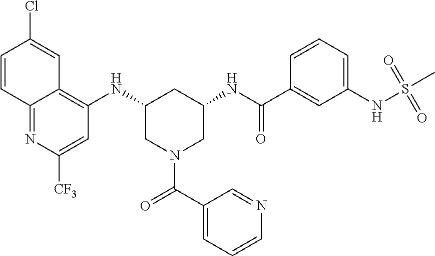 [SMILES: CS(=O)(=O)Nc1cccc(C(=O)N[C@H]2C[C@@H](Nc3cc(C(F)(F)F)nc4ccc(Cl)cc34)CN(C(=O)c3cccnc3)C2)c1] |
| 20 | 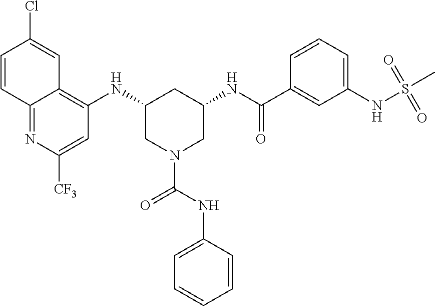 [SMILES: CS(=O)(=O)Nc1cccc(C(=O)N[C@H]2C[C@@H](Nc3cc(C(F)(F)F)nc4ccc(Cl)cc34)CN(C(=O)Nc3ccccc3)C2)c1] |
| 21 | 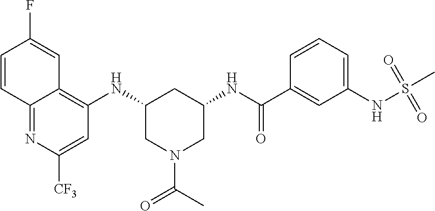 [SMILES: CC(=O)N1C[C@@H](NC(=O)c2cccc(NS(C)(=O)=O)c2)C[C@@H](Nc2cc(C(F)(F)F)nc3ccc(F)cc23)C1] |
| 22 | 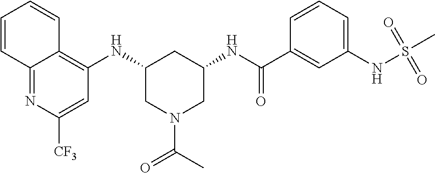 [SMILES: CC(=O)N1C[C@@H](NC(=O)c2cccc(NS(C)(=O)=O)c2)C[C@@H](Nc2cc(C(F)(F)F)nc3ccccc23)C1] |
| 23 | 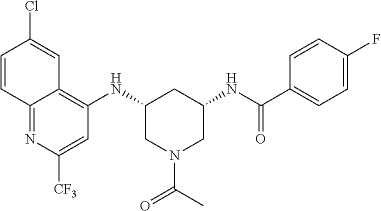 [SMILES: CC(=O)N1C[C@@H](NC(=O)c2ccc(F)cc2)C[C@@H](Nc2cc(C(F)(F)F)nc3ccc(Cl)cc23)C1] |
| 24 | 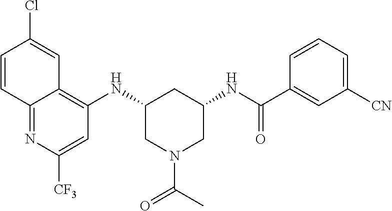 [SMILES: CC(=O)N1C[C@@H](NC(=O)c2cccc(C#N)c2)C[C@@H](Nc2cc(C(F)(F)F)nc3ccc(Cl)cc23)C1] |
| 25 | 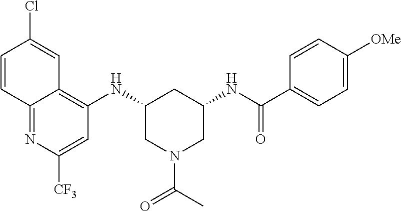 [SMILES: COc1ccc(C(=O)N[C@H]2C[C@@H](Nc3cc(C(F)(F)F)nc4ccc(Cl)cc34)CN(C(C)=O)C2)cc1] |
| 26 | 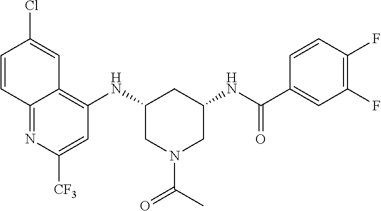 [SMILES: CC(=O)N1C[C@@H](NC(=O)c2ccc(F)c(F)c2)C[C@@H](Nc2cc(C(F)(F)F)nc3ccc(Cl)cc23)C1] |
| 27 | 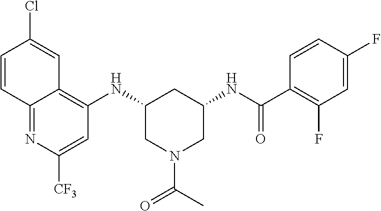 [SMILES: CC(=O)N1C[C@@H](NC(=O)c2ccc(F)cc2F)C[C@@H](Nc2cc(C(F)(F)F)nc3ccc(Cl)cc23)C1] |
| 28 | 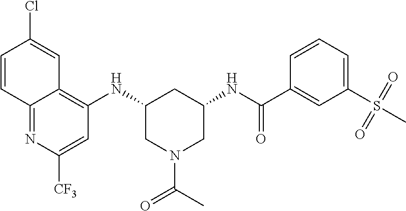 [SMILES: CC(=O)N1C[C@@H](NC(=O)c2cccc(S(C)(=O)=O)c2)C[C@@H](Nc2cc(C(F)(F)F)nc3ccc(Cl)cc23)C1] |
| 29 | 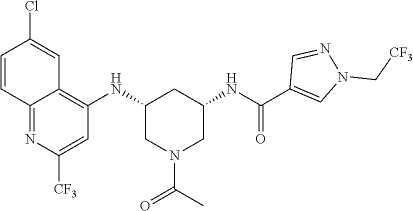 [SMILES: CC(=O)N1C[C@@H](NC(=O)c2cnn(CC(F)(F)F)c2)C[C@@H](Nc2cc(C(F)(F)F)nc3ccc(Cl)cc23)C1] |
| 30 | 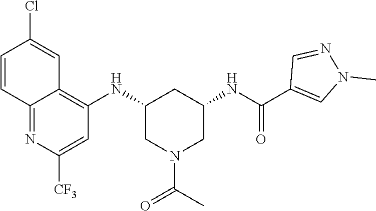 [SMILES: CC(=O)N1C[C@@H](NC(=O)c2cnn(C)c2)C[C@@H](Nc2cc(C(F)(F)F)nc3ccc(Cl)cc23)C1] |
| 31 | 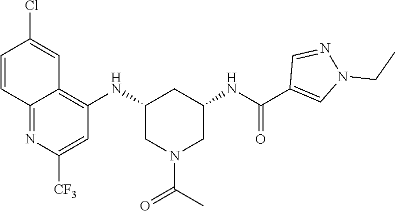 [SMILES: CCn1cc(C(=O)N[C@H]2C[C@@H](Nc3cc(C(F)(F)F)nc4ccc(Cl)cc34)CN(C(C)=O)C2)cn1] |
| 32 | 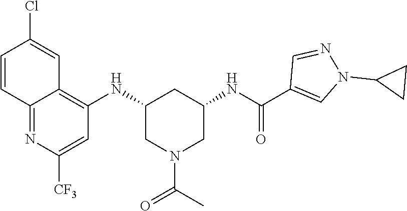 [SMILES: CC(=O)N1C[C@@H](NC(=O)c2cnn(C3CC3)c2)C[C@@H](Nc2cc(C(F)(F)F)nc3ccc(Cl)cc23)C1] |
| 33 | 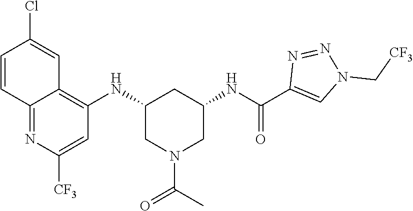 [SMILES: CC(=O)N1C[C@@H](NC(=O)c2cn(CC(F)(F)F)nn2)C[C@@H](Nc2cc(C(F)(F)F)nc3ccc(Cl)cc23)C1] |
| 34 | 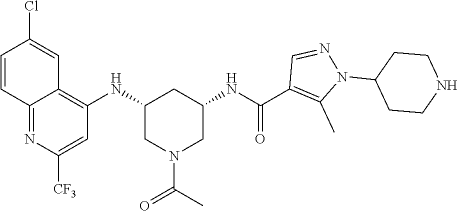 [SMILES: CC(=O)N1C[C@@H](NC(=O)c2cnn(C3CCNCC3)c2C)C[C@@H](Nc2cc(C(F)(F)F)nc3ccc(Cl)cc23)C1] |
| 35 | 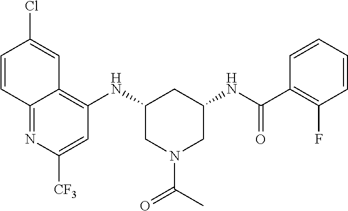 [SMILES: CC(=O)N1C[C@@H](NC(=O)c2ccccc2F)C[C@@H](Nc2cc(C(F)(F)F)nc3ccc(Cl)cc23)C1] |
| 36 | 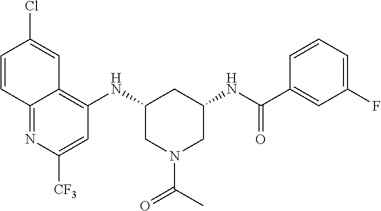 [SMILES: CC(=O)N1C[C@@H](NC(=O)c2cccc(F)c2)C[C@@H](Nc2cc(C(F)(F)F)nc3ccc(Cl)cc23)C1] |
| 37 | 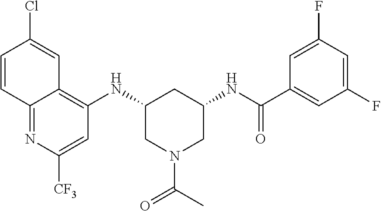 [SMILES: CC(=O)N1C[C@@H](NC(=O)c2cc(F)cc(F)c2)C[C@@H](Nc2cc(C(F)(F)F)nc3ccc(Cl)cc23)C1] |
| 38 | 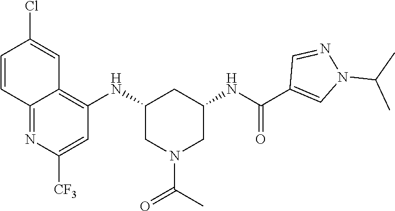 [SMILES: CC(=O)N1C[C@@H](NC(=O)c2cnn(C(C)C)c2)C[C@@H](Nc2cc(C(F)(F)F)nc3ccc(Cl)cc23)C1] |
| 39 | 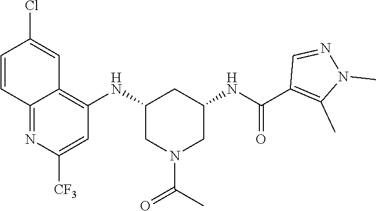 [SMILES: CC(=O)N1C[C@@H](NC(=O)c2cnn(C)c2C)C[C@@H](Nc2cc(C(F)(F)F)nc3ccc(Cl)cc23)C1] |
| 40 | 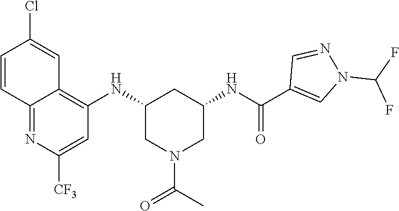 [SMILES: CC(=O)N1C[C@@H](NC(=O)c2cnn(C(F)F)c2)C[C@@H](Nc2cc(C(F)(F)F)nc3ccc(Cl)cc23)C1] |
| 41 | 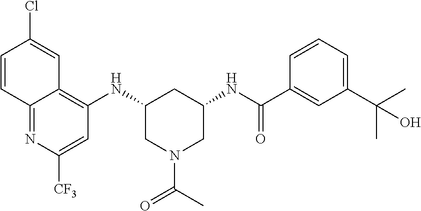 [SMILES: CC(=O)N1C[C@@H](NC(=O)c2cccc(C(C)(C)O)c2)C[C@@H](Nc2cc(C(F)(F)F)nc3ccc(Cl)cc23)C1] |
| 42 | 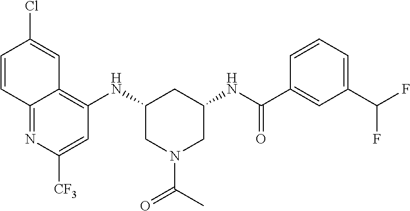 [SMILES: CC(=O)N1C[C@@H](NC(=O)c2cccc(C(F)F)c2)C[C@@H](Nc2cc(C(F)(F)F)nc3ccc(Cl)cc23)C1] |
| 43 | 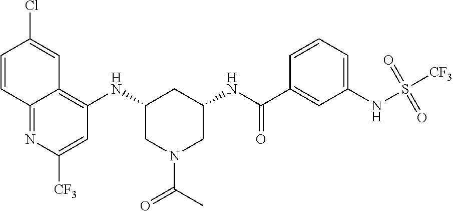 [SMILES: CC(=O)N1C[C@@H](NC(=O)c2cccc(NS(=O)(=O)C(F)(F)F)c2)C[C@@H](Nc2cc(C(F)(F)F)nc3ccc(Cl)cc23)C1] |
| 44 | 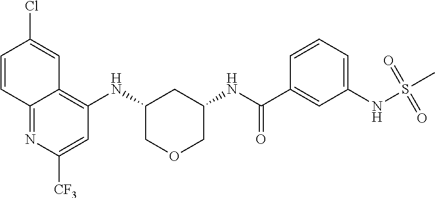 [SMILES: CS(=O)(=O)Nc1cccc(C(=O)N[C@@H]2COC[C@H](Nc3cc(C(F)(F)F)nc4ccc(Cl)cc34)C2)c1] |
| 45 | 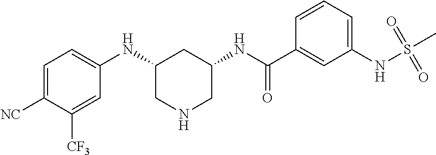 [SMILES: CS(=O)(=O)Nc1cccc(C(=O)N[C@@H]2CNC[C@H](Nc3ccc(C#N)c(C(F)(F)F)c3)C2)c1] |
| 46 | 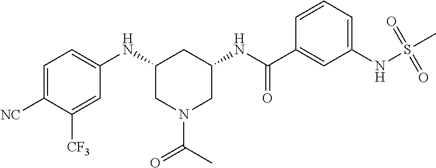 [SMILES: CC(=O)N1C[C@@H](NC(=O)c2cccc(NS(C)(=O)=O)c2)C[C@@H](Nc2ccc(C#N)c(C(F)(F)F)c2)C1] |
| 47 | 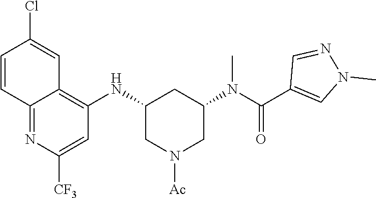 [SMILES: CC(=O)N1C[C@H](Nc2cc(C(F)(F)F)nc3ccc(Cl)cc23)C[C@H](N(C)C(=O)c2cnn(C)c2)C1] |
| 48 | 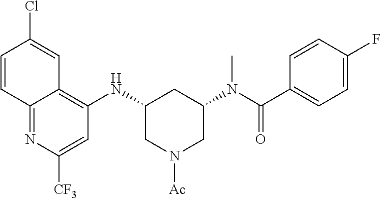 [SMILES: CC(=O)N1C[C@H](Nc2cc(C(F)(F)F)nc3ccc(Cl)cc23)C[C@H](N(C)C(=O)c2ccc(F)cc2)C1] |
| 49 | 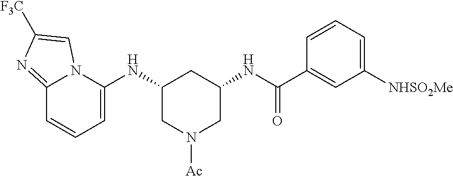 |
| 50 | 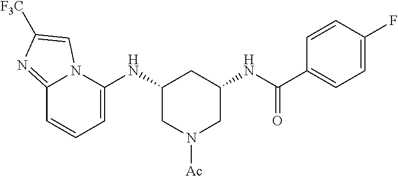 [SMILES: CC(=O)N1C[C@@H](NC(=O)c2ccc(F)cc2)C[C@@H](Nc2cccc3nc(C(F)(F)F)cn23)C1] |
| 51 | 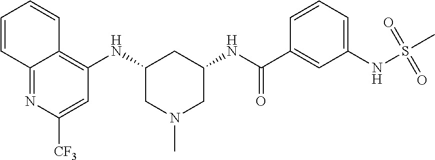 [SMILES: CN1C[C@@H](NC(=O)c2cccc(NS(C)(=O)=O)c2)C[C@@H](Nc2cc(C(F)(F)F)nc3ccccc23)C1] |
| 52 | 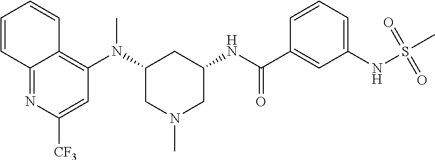 [SMILES: CN1C[C@@H](NC(=O)c2cccc(NS(C)(=O)=O)c2)C[C@@H](N(C)c2cc(C(F)(F)F)nc3ccccc23)C1] |
| 53 | 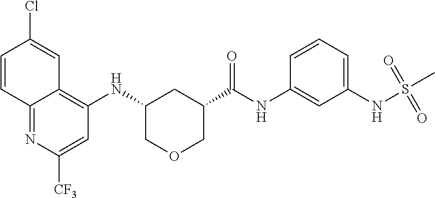 [SMILES: CS(=O)(=O)Nc1cccc(NC(=O)[C@@H]2COC[C@H](Nc3cc(C(F)(F)F)nc4ccc(Cl)cc34)C2)c1] |
| 54 | 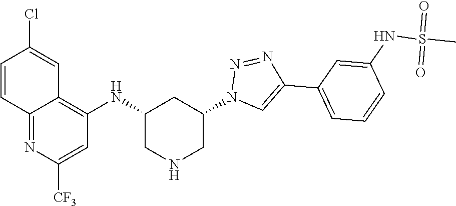 [SMILES: CS(=O)(=O)Nc1cccc(-c2cn([C@@H]3CNC[C@H](Nc4cc(C(F)(F)F)nc5ccc(Cl)cc45)C3)nn2)c1] |
| 55 | 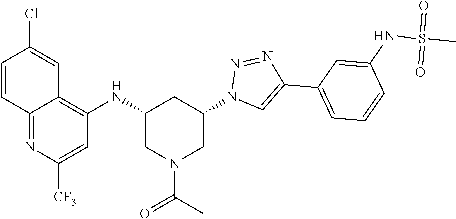 [SMILES: CC(=O)N1C[C@H](Nc2cc(C(F)(F)F)nc3ccc(Cl)cc23)C[C@H](n2cc(-c3cccc(NS(C)(=O)=O)c3)nn2)C1] |
| 56 | 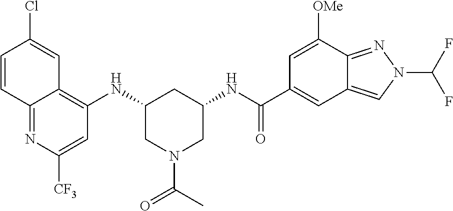 [SMILES: COc1cc(C(=O)N[C@H]2C[C@@H](Nc3cc(C(F)(F)F)nc4ccc(Cl)cc34)CN(C(C)=O)C2)cc2cn(C(F)F)nc12] |
| 57 | 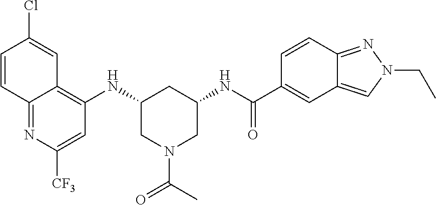 [SMILES: CCn1cc2cc(C(=O)N[C@H]3C[C@@H](Nc4cc(C(F)(F)F)nc5ccc(Cl)cc45)CN(C(C)=O)C3)ccc2n1] |
| 58 | 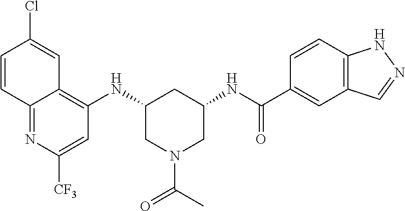 [SMILES: CC(=O)N1C[C@@H](NC(=O)c2ccc3[nH]ncc3c2)C[C@@H](Nc2cc(C(F)(F)F)nc3ccc(Cl)cc23)C1] |
| 59 | 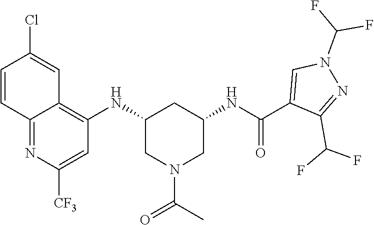 [SMILES: CC(=O)N1C[C@@H](NC(=O)c2cn(C(F)F)nc2C(F)F)C[C@@H](Nc2cc(C(F)(F)F)nc3ccc(Cl)cc23)C1] |
| 60 | 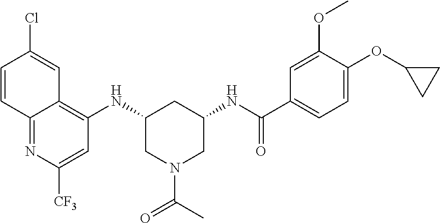 [SMILES: COc1cc(C(=O)N[C@H]2C[C@@H](Nc3cc(C(F)(F)F)nc4ccc(Cl)cc34)CN(C(C)=O)C2)ccc1OC1CC1] |
| 61 | 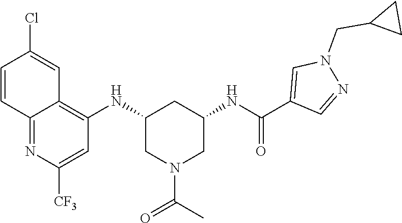 [SMILES: CC(=O)N1C[C@@H](NC(=O)c2cnn(CC3CC3)c2)C[C@@H](Nc2cc(C(F)(F)F)nc3ccc(Cl)cc23)C1] |
| 62 | 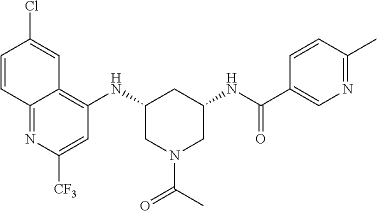 [SMILES: CC(=O)N1C[C@@H](NC(=O)c2ccc(C)nc2)C[C@@H](Nc2cc(C(F)(F)F)nc3ccc(Cl)cc23)C1] |
| 63 | 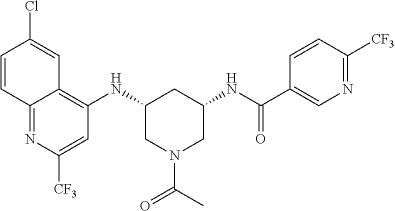 [SMILES: CC(=O)N1C[C@@H](NC(=O)c2ccc(C(F)(F)F)nc2)C[C@@H](Nc2cc(C(F)(F)F)nc3ccc(Cl)cc23)C1] |
| 64 | 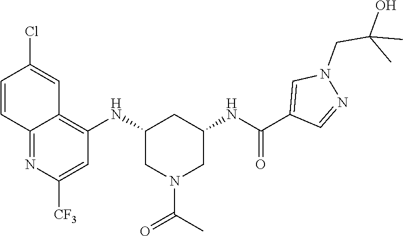 [SMILES: CC(=O)N1C[C@@H](NC(=O)c2cnn(CC(C)(C)O)c2)C[C@@H](Nc2cc(C(F)(F)F)nc3ccc(Cl)cc23)C1] |
| 65 | 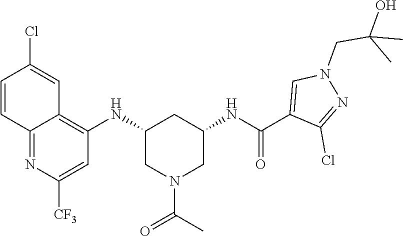 [SMILES: CC(=O)N1C[C@@H](NC(=O)c2cn(CC(C)(C)O)nc2Cl)C[C@@H](Nc2cc(C(F)(F)F)nc3ccc(Cl)cc23)C1] |
| 66 | 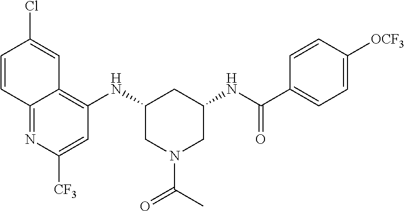 [SMILES: CC(=O)N1C[C@@H](NC(=O)c2ccc(OC(F)(F)F)cc2)C[C@@H](Nc2cc(C(F)(F)F)nc3ccc(Cl)cc23)C1] |
| 67 | 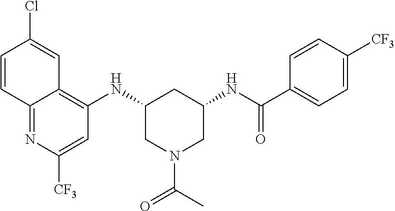 [SMILES: CC(=O)N1C[C@@H](NC(=O)c2ccc(C(F)(F)F)cc2)C[C@@H](Nc2cc(C(F)(F)F)nc3ccc(Cl)cc23)C1] |
| 68 | 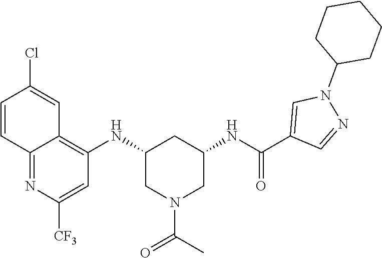 [SMILES: CC(=O)N1C[C@@H](NC(=O)c2cnn(C3CCCCC3)c2)C[C@@H](Nc2cc(C(F)(F)F)nc3ccc(Cl)cc23)C1] |
| 69 | 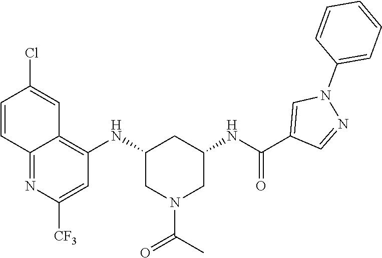 [SMILES: CC(=O)N1C[C@@H](NC(=O)c2cnn(-c3ccccc3)c2)C[C@@H](Nc2cc(C(F)(F)F)nc3ccc(Cl)cc23)C1] |
| 70 | 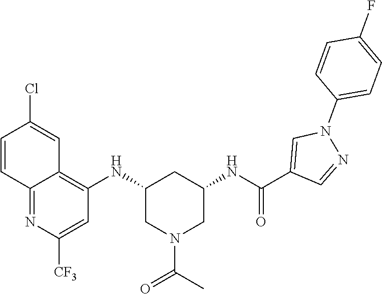 [SMILES: CC(=O)N1C[C@@H](NC(=O)c2cnn(-c3ccc(F)cc3)c2)C[C@@H](Nc2cc(C(F)(F)F)nc3ccc(Cl)cc23)C1] |
| 71 | 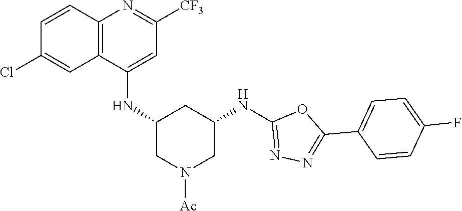 [SMILES: CC(=O)N1C[C@@H](Nc2nnc(-c3ccc(F)cc3)o2)C[C@@H](Nc2cc(C(F)(F)F)nc3ccc(Cl)cc23)C1] |
| 72 | 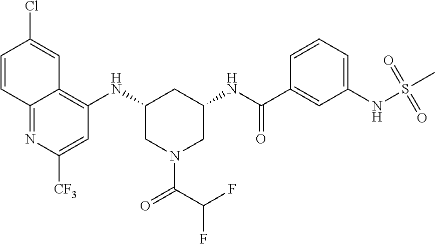 [SMILES: CS(=O)(=O)Nc1cccc(C(=O)N[C@H]2C[C@@H](Nc3cc(C(F)(F)F)nc4ccc(Cl)cc34)CN(C(=O)C(F)F)C2)c1] |
| 73 | 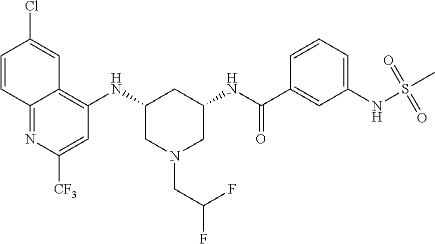 [SMILES: CS(=O)(=O)Nc1cccc(C(=O)N[C@H]2C[C@@H](Nc3cc(C(F)(F)F)nc4ccc(Cl)cc34)CN(CC(F)F)C2)c1] |
| 74 | 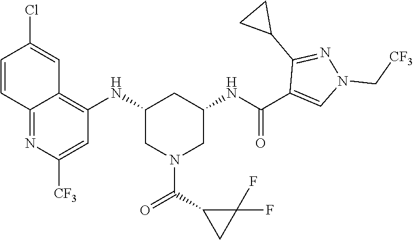 [SMILES: O=C(N[C@H]1C[C@@H](Nc2cc(C(F)(F)F)nc3ccc(Cl)cc23)CN(C(=O)[C@H]2CC2(F)F)C1)c1cn(CC(F)(F)F)nc1C1CC1] |
| 75 | 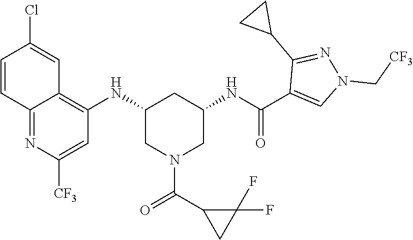 [SMILES: O=C(N[C@H]1C[C@@H](Nc2cc(C(F)(F)F)nc3ccc(Cl)cc23)CN(C(=O)C2CC2(F)F)C1)c1cn(CC(F)(F)F)nc1C1CC1] |
| 76 | 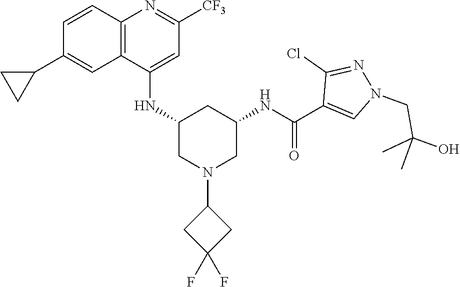 [SMILES: CC(C)(O)Cn1cc(C(=O)N[C@H]2C[C@@H](Nc3cc(C(F)(F)F)nc4ccc(C5CC5)cc34)CN(C3CC(F)(F)C3)C2)c(Cl)n1] |
| 77 | 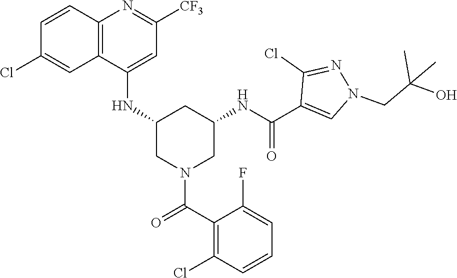 [SMILES: CC(C)(O)Cn1cc(C(=O)N[C@H]2C[C@@H](Nc3cc(C(F)(F)F)nc4ccc(Cl)cc34)CN(C(=O)c3c(F)cccc3Cl)C2)c(Cl)n1] |
| 78 | 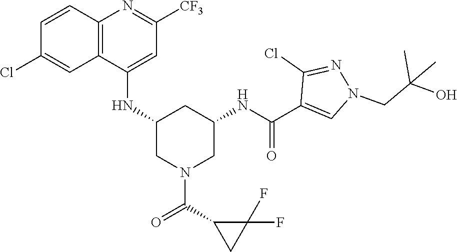 [SMILES: CC(C)(O)Cn1cc(C(=O)N[C@H]2C[C@@H](Nc3cc(C(F)(F)F)nc4ccc(Cl)cc34)CN(C(=O)[C@H]3CC3(F)F)C2)c(Cl)n1] |
| 79 | 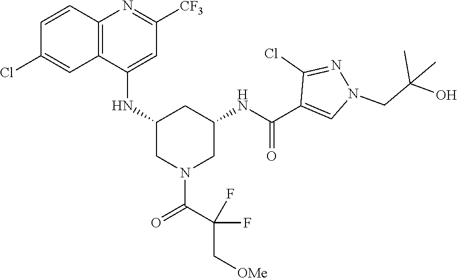 [SMILES: COCC(F)(F)C(=O)N1C[C@@H](NC(=O)c2cn(CC(C)(C)O)nc2Cl)C[C@@H](Nc2cc(C(F)(F)F)nc3ccc(Cl)cc23)C1] |
| 80 | 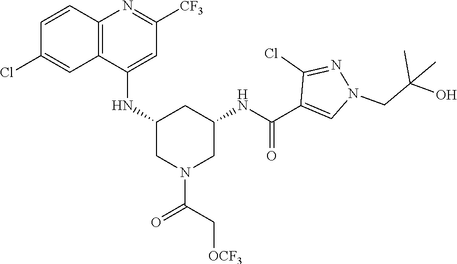 [SMILES: CC(C)(O)Cn1cc(C(=O)N[C@H]2C[C@@H](Nc3cc(C(F)(F)F)nc4ccc(Cl)cc34)CN(C(=O)COC(F)(F)F)C2)c(Cl)n1] |
| 81 | 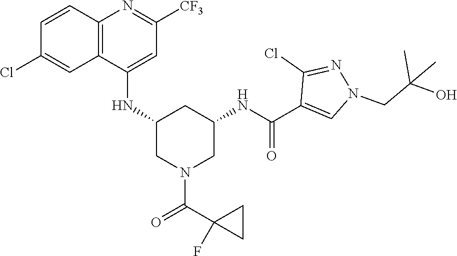 [SMILES: CC(C)(O)Cn1cc(C(=O)N[C@H]2C[C@@H](Nc3cc(C(F)(F)F)nc4ccc(Cl)cc34)CN(C(=O)C3(F)CC3)C2)c(Cl)n1] |
| 82 | 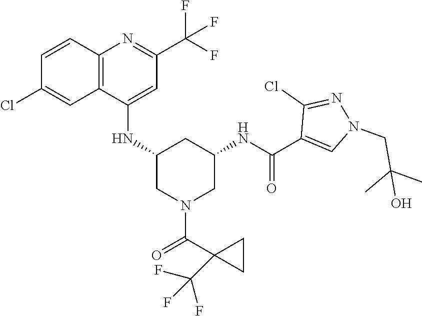 [SMILES: CC(C)(O)Cn1cc(C(=O)N[C@H]2C[C@@H](Nc3cc(C(F)(F)F)nc4ccc(Cl)cc34)CN(C(=O)C3(C(F)(F)F)CC3)C2)c(Cl)n1] |
| 83 | 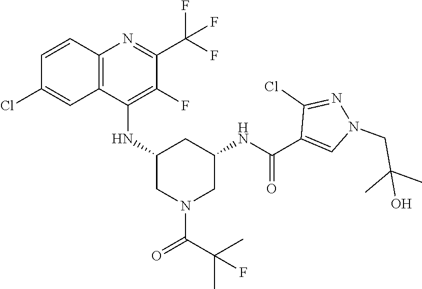 [SMILES: CC(C)(O)Cn1cc(C(=O)N[C@H]2C[C@@H](Nc3c(F)c(C(F)(F)F)nc4ccc(Cl)cc34)CN(C(=O)C(C)(C)F)C2)c(Cl)n1] |
| 84 | 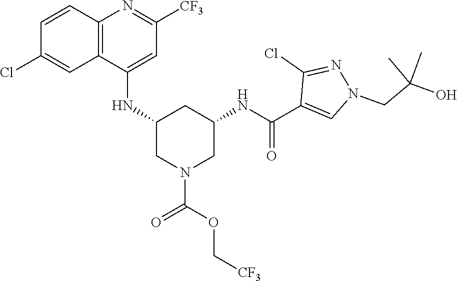 [SMILES: CC(C)(O)Cn1cc(C(=O)N[C@H]2C[C@@H](Nc3cc(C(F)(F)F)nc4ccc(Cl)cc34)CN(C(=O)OCC(F)(F)F)C2)c(Cl)n1] |
| 85 | 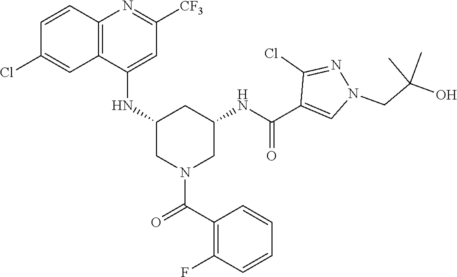 [SMILES: CC(C)(O)Cn1cc(C(=O)N[C@H]2C[C@@H](Nc3cc(C(F)(F)F)nc4ccc(Cl)cc34)CN(C(=O)c3ccccc3F)C2)c(Cl)n1] |
| 86 | 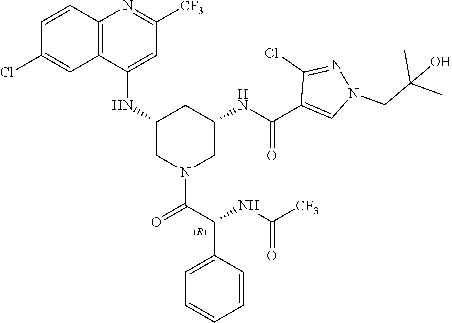 |
| 87 | 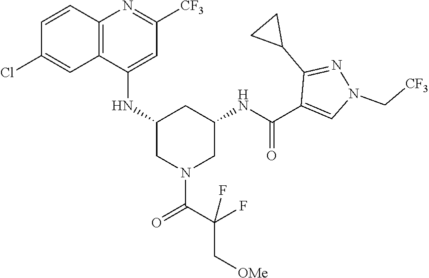 [SMILES: COCC(F)(F)C(=O)N1C[C@@H](NC(=O)c2cn(CC(F)(F)F)nc2C2CC2)C[C@@H](Nc2cc(C(F)(F)F)nc3ccc(Cl)cc23)C1] |
| 88 | 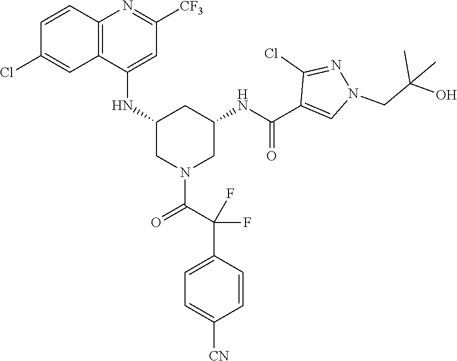 [SMILES: CC(C)(O)Cn1cc(C(=O)N[C@H]2C[C@@H](Nc3cc(C(F)(F)F)nc4ccc(Cl)cc34)CN(C(=O)C(F)(F)c3ccc(C#N)cc3)C2)c(Cl)n1] |
| 89 | 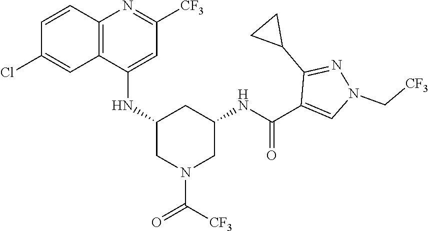 [SMILES: O=C(N[C@H]1C[C@@H](Nc2cc(C(F)(F)F)nc3ccc(Cl)cc23)CN(C(=O)C(F)(F)F)C1)c1cn(CC(F)(F)F)nc1C1CC1] |
| 90 | 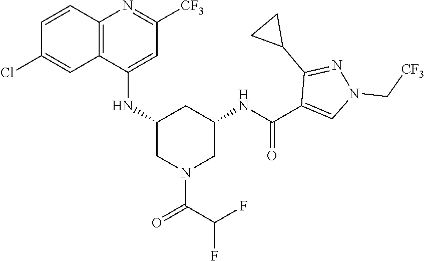 [SMILES: O=C(N[C@H]1C[C@@H](Nc2cc(C(F)(F)F)nc3ccc(Cl)cc23)CN(C(=O)C(F)F)C1)c1cn(CC(F)(F)F)nc1C1CC1] |
| 91 | 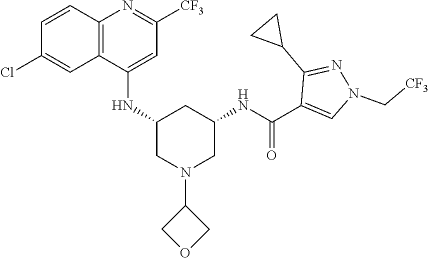 [SMILES: O=C(N[C@H]1C[C@@H](Nc2cc(C(F)(F)F)nc3ccc(Cl)cc23)CN(C2COC2)C1)c1cn(CC(F)(F)F)nc1C1CC1] |
| 92 | 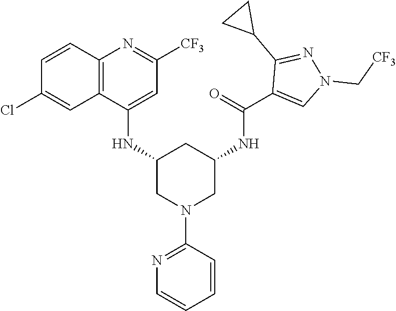 [SMILES: O=C(N[C@H]1C[C@@H](Nc2cc(C(F)(F)F)nc3ccc(Cl)cc23)CN(c2ccccn2)C1)c1cn(CC(F)(F)F)nc1C1CC1] |
| 93 | 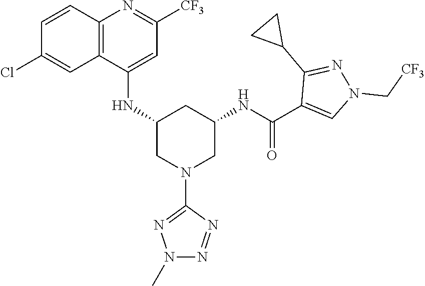 [SMILES: Cn1nnc(N2C[C@@H](NC(=O)c3cn(CC(F)(F)F)nc3C3CC3)C[C@@H](Nc3cc(C(F)(F)F)nc4ccc(Cl)cc34)C2)n1] |
| 94 | 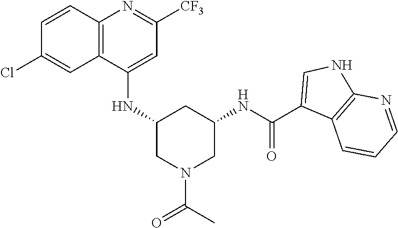 [SMILES: CC(=O)N1C[C@@H](NC(=O)c2c[nH]c3ncccc23)C[C@@H](Nc2cc(C(F)(F)F)nc3ccc(Cl)cc23)C1] |
| 95 | 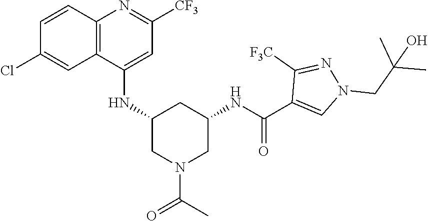 [SMILES: CC(=O)N1C[C@@H](NC(=O)c2cn(CC(C)(C)O)nc2C(F)(F)F)C[C@@H](Nc2cc(C(F)(F)F)nc3ccc(Cl)cc23)C1] |
| 96 | 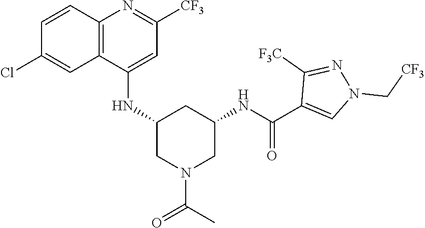 [SMILES: CC(=O)N1C[C@@H](NC(=O)c2cn(CC(F)(F)F)nc2C(F)(F)F)C[C@@H](Nc2cc(C(F)(F)F)nc3ccc(Cl)cc23)C1] |
| 97 | 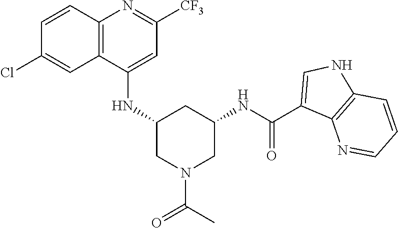 [SMILES: CC(=O)N1C[C@@H](NC(=O)c2c[nH]c3cccnc23)C[C@@H](Nc2cc(C(F)(F)F)nc3ccc(Cl)cc23)C1] |
| 98 | 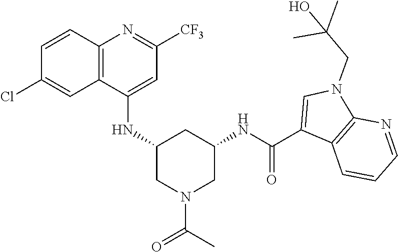 [SMILES: CC(=O)N1C[C@@H](NC(=O)c2cn(CC(C)(C)O)c3ncccc23)C[C@@H](Nc2cc(C(F)(F)F)nc3ccc(Cl)cc23)C1] |
| 99 | 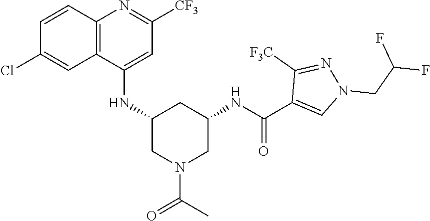 [SMILES: CC(=O)N1C[C@@H](NC(=O)c2cn(CC(F)F)nc2C(F)(F)F)C[C@@H](Nc2cc(C(F)(F)F)nc3ccc(Cl)cc23)C1] |
| 100 | 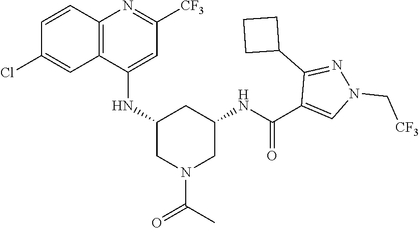 [SMILES: CC(=O)N1C[C@@H](NC(=O)c2cn(CC(F)(F)F)nc2C2CCC2)C[C@@H](Nc2cc(C(F)(F)F)nc3ccc(Cl)cc23)C1] |
| 101 | 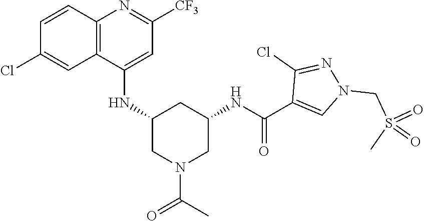 [SMILES: CC(=O)N1C[C@@H](NC(=O)c2cn(CS(C)(=O)=O)nc2Cl)C[C@@H](Nc2cc(C(F)(F)F)nc3ccc(Cl)cc23)C1] |
| 102 | 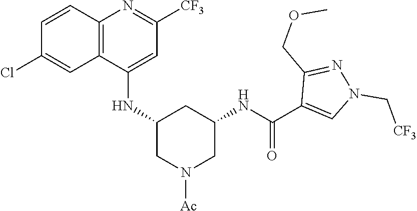 [SMILES: COCc1nn(CC(F)(F)F)cc1C(=O)N[C@H]1C[C@@H](Nc2cc(C(F)(F)F)nc3ccc(Cl)cc23)CN(C(C)=O)C1] |
| 103 | 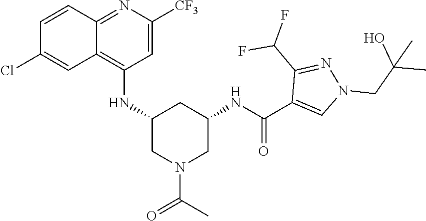 [SMILES: CC(=O)N1C[C@@H](NC(=O)c2cn(CC(C)(C)O)nc2C(F)F)C[C@@H](Nc2cc(C(F)(F)F)nc3ccc(Cl)cc23)C1] |
| 104 | 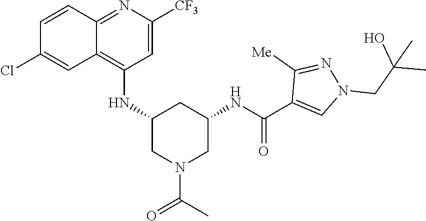 [SMILES: CC(=O)N1C[C@@H](NC(=O)c2cn(CC(C)(C)O)nc2C)C[C@@H](Nc2cc(C(F)(F)F)nc3ccc(Cl)cc23)C1] |
| 105 | 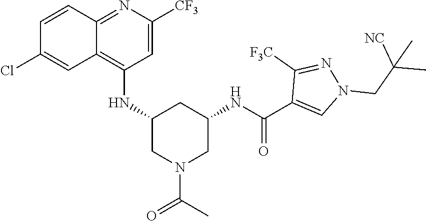 [SMILES: CC(=O)N1C[C@@H](NC(=O)c2cn(CC(C)(C)C#N)nc2C(F)(F)F)C[C@@H](Nc2cc(C(F)(F)F)nc3ccc(Cl)cc23)C1] |
| 106 | 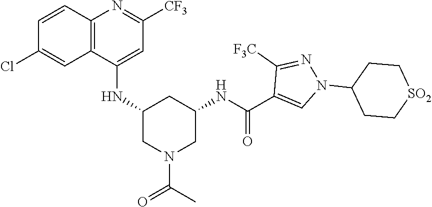 [SMILES: CC(=O)N1C[C@@H](NC(=O)c2cn(C3CCS(=O)(=O)CC3)nc2C(F)(F)F)C[C@@H](Nc2cc(C(F)(F)F)nc3ccc(Cl)cc23)C1] |
| 107 | 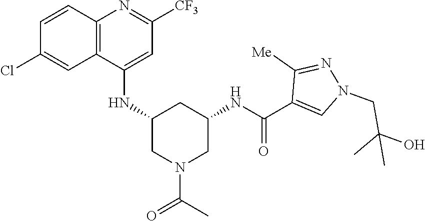 [SMILES: CC(=O)N1C[C@@H](NC(=O)c2cn(CC(C)(C)O)nc2C)C[C@@H](Nc2cc(C(F)(F)F)nc3ccc(Cl)cc23)C1] |
| 108 | 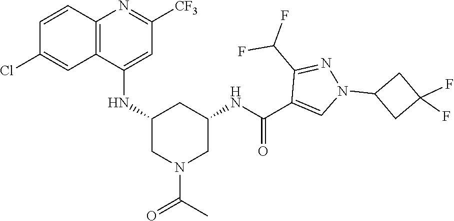 [SMILES: CC(=O)N1C[C@@H](NC(=O)c2cn(C3CC(F)(F)C3)nc2C(F)F)C[C@@H](Nc2cc(C(F)(F)F)nc3ccc(Cl)cc23)C1] |
| 109 | 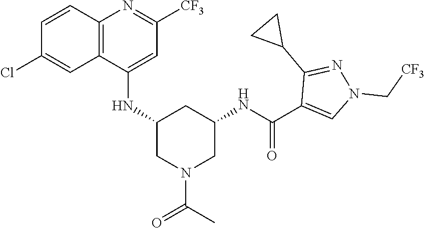 [SMILES: CC(=O)N1C[C@@H](NC(=O)c2cn(CC(F)(F)F)nc2C2CC2)C[C@@H](Nc2cc(C(F)(F)F)nc3ccc(Cl)cc23)C1] |
| 110 | 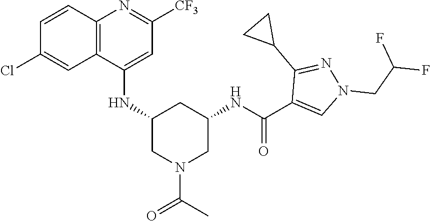 [SMILES: CC(=O)N1C[C@@H](NC(=O)c2cn(CC(F)F)nc2C2CC2)C[C@@H](Nc2cc(C(F)(F)F)nc3ccc(Cl)cc23)C1] |
| 111 | 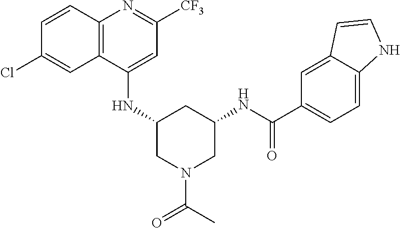 [SMILES: CC(=O)N1C[C@@H](NC(=O)c2ccc3[nH]ccc3c2)C[C@@H](Nc2cc(C(F)(F)F)nc3ccc(Cl)cc23)C1] |
| 112 | 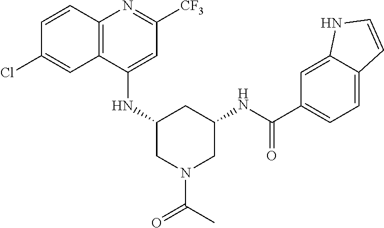 [SMILES: CC(=O)N1C[C@@H](NC(=O)c2ccc3cc[nH]c3c2)C[C@@H](Nc2cc(C(F)(F)F)nc3ccc(Cl)cc23)C1] |
| 113 | 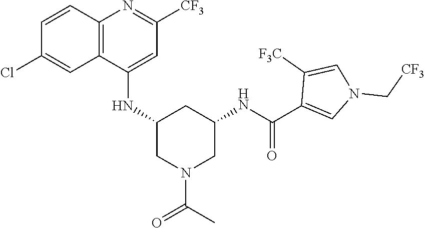 [SMILES: CC(=O)N1C[C@@H](NC(=O)c2cn(CC(F)(F)F)cc2C(F)(F)F)C[C@@H](Nc2cc(C(F)(F)F)nc3ccc(Cl)cc23)C1] |
| 114 | 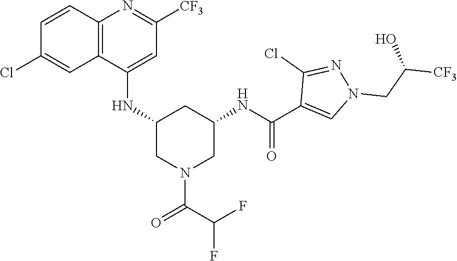 [SMILES: O=C(N[C@H]1C[C@@H](Nc2cc(C(F)(F)F)nc3ccc(Cl)cc23)CN(C(=O)C(F)F)C1)c1cn(C[C@H](O)C(F)(F)F)nc1Cl] |
| 115 | 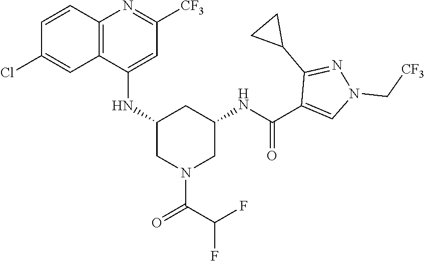 [SMILES: O=C(N[C@H]1C[C@@H](Nc2cc(C(F)(F)F)nc3ccc(Cl)cc23)CN(C(=O)C(F)F)C1)c1cn(CC(F)(F)F)nc1C1CC1] |
| 116 | 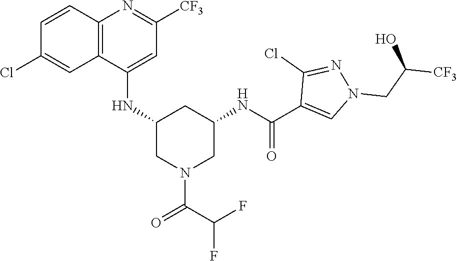 [SMILES: O=C(N[C@H]1C[C@@H](Nc2cc(C(F)(F)F)nc3ccc(Cl)cc23)CN(C(=O)C(F)F)C1)c1cn(C[C@@H](O)C(F)(F)F)nc1Cl] |
| 117 | 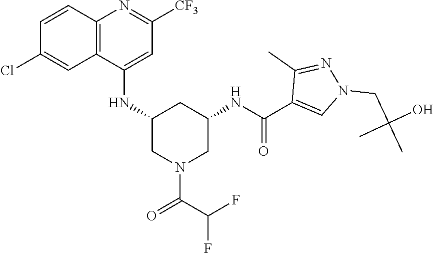 [SMILES: Cc1nn(CC(C)(C)O)cc1C(=O)N[C@H]1C[C@@H](Nc2cc(C(F)(F)F)nc3ccc(Cl)cc23)CN(C(=O)C(F)F)C1] |
| 118 | 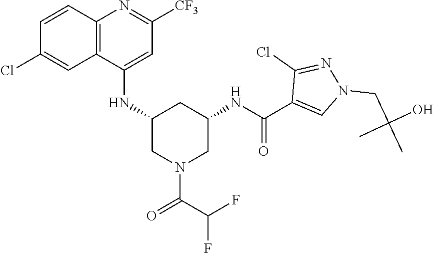 [SMILES: CC(C)(O)Cn1cc(C(=O)N[C@H]2C[C@@H](Nc3cc(C(F)(F)F)nc4ccc(Cl)cc34)CN(C(=O)C(F)F)C2)c(Cl)n1] |
| 119 | 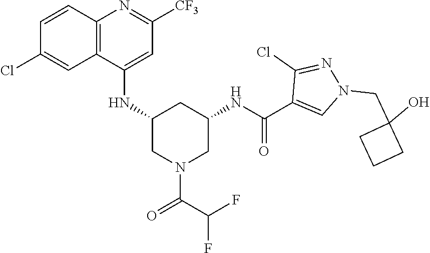 [SMILES: O=C(N[C@H]1C[C@@H](Nc2cc(C(F)(F)F)nc3ccc(Cl)cc23)CN(C(=O)C(F)F)C1)c1cn(CC2(O)CCC2)nc1Cl] |
| 120 | 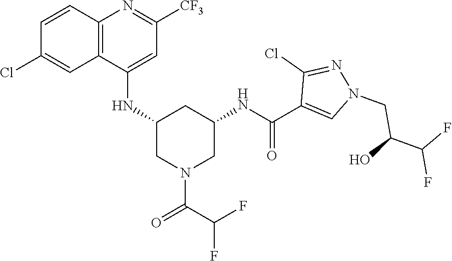 [SMILES: O=C(N[C@H]1C[C@@H](Nc2cc(C(F)(F)F)nc3ccc(Cl)cc23)CN(C(=O)C(F)F)C1)c1cn(C[C@H](O)C(F)F)nc1Cl] |
| 121 | 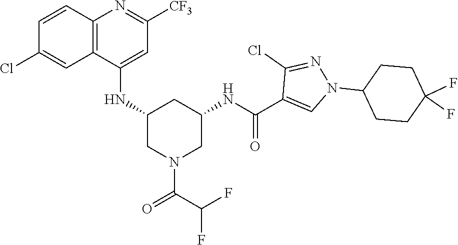 [SMILES: O=C(N[C@H]1C[C@@H](Nc2cc(C(F)(F)F)nc3ccc(Cl)cc23)CN(C(=O)C(F)F)C1)c1cn(C2CCC(F)(F)CC2)nc1Cl] |
| 122 | 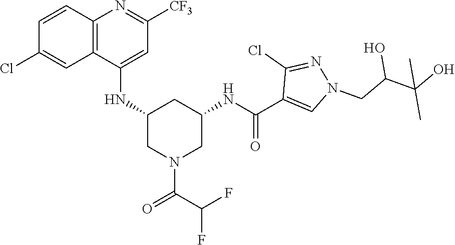 [SMILES: CC(C)(O)C(O)Cn1cc(C(=O)N[C@H]2C[C@@H](Nc3cc(C(F)(F)F)nc4ccc(Cl)cc34)CN(C(=O)C(F)F)C2)c(Cl)n1] |
| 123 | 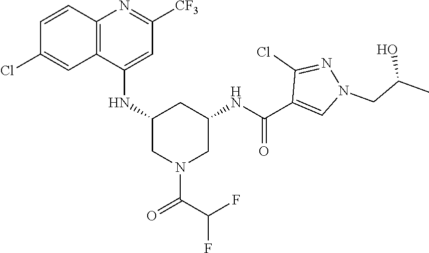 [SMILES: C[C@@H](O)Cn1cc(C(=O)N[C@H]2C[C@@H](Nc3cc(C(F)(F)F)nc4ccc(Cl)cc34)CN(C(=O)C(F)F)C2)c(Cl)n1] |
| 124 | 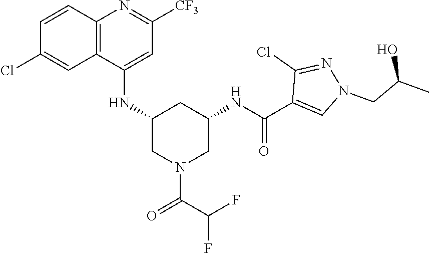 [SMILES: C[C@H](O)Cn1cc(C(=O)N[C@H]2C[C@@H](Nc3cc(C(F)(F)F)nc4ccc(Cl)cc34)CN(C(=O)C(F)F)C2)c(Cl)n1] |
| 125 | 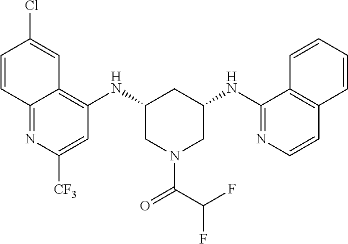 [SMILES: O=C(C(F)F)N1C[C@@H](Nc2nccc3ccccc23)C[C@@H](Nc2cc(C(F)(F)F)nc3ccc(Cl)cc23)C1] |
| 126 | 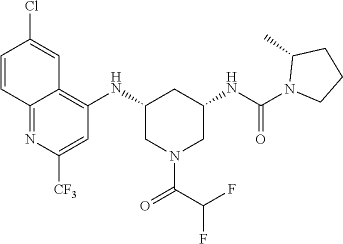 [SMILES: C[C@@H]1CCCN1C(=O)N[C@H]1C[C@@H](Nc2cc(C(F)(F)F)nc3ccc(Cl)cc23)CN(C(=O)C(F)F)C1] |
| 127 | 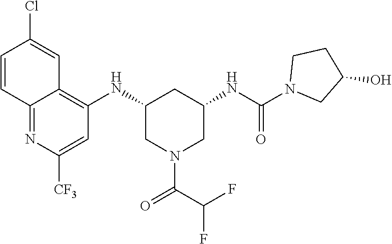 [SMILES: O=C(N[C@H]1C[C@@H](Nc2cc(C(F)(F)F)nc3ccc(Cl)cc23)CN(C(=O)C(F)F)C1)N1CC[C@H](O)C1] |
| 128 | 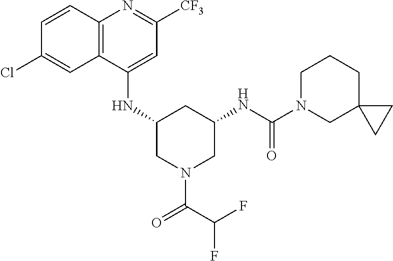 [SMILES: O=C(N[C@H]1C[C@@H](Nc2cc(C(F)(F)F)nc3ccc(Cl)cc23)CN(C(=O)C(F)F)C1)N1CCCC2(CC2)C1] |
| 129 | 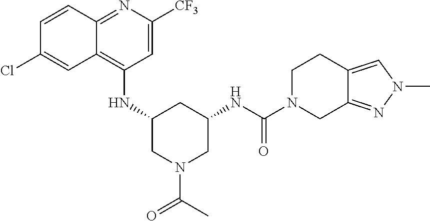 [SMILES: CC(=O)N1C[C@@H](NC(=O)N2CCc3cn(C)nc3C2)C[C@@H](Nc2cc(C(F)(F)F)nc3ccc(Cl)cc23)C1] |
| 130 | 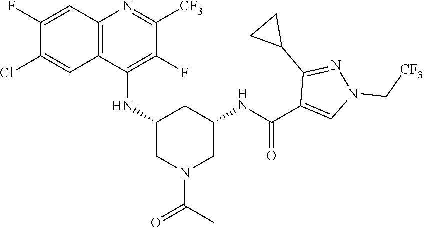 [SMILES: CC(=O)N1C[C@@H](NC(=O)c2cn(CC(F)(F)F)nc2C2CC2)C[C@@H](Nc2c(F)c(C(F)(F)F)nc3cc(F)c(Cl)cc23)C1] |
| 131 | 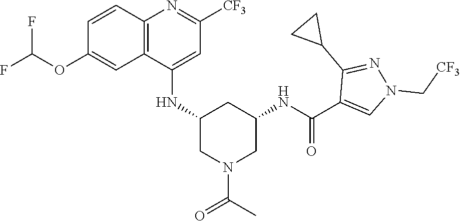 [SMILES: CC(=O)N1C[C@@H](NC(=O)c2cn(CC(F)(F)F)nc2C2CC2)C[C@@H](Nc2cc(C(F)(F)F)nc3ccc(OC(F)F)cc23)C1] |
| 132 | 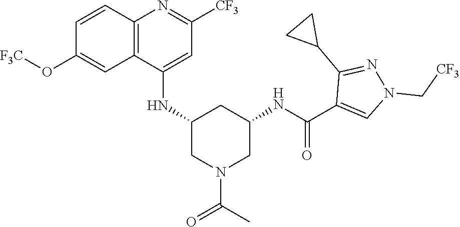 [SMILES: CC(=O)N1C[C@@H](NC(=O)c2cn(CC(F)(F)F)nc2C2CC2)C[C@@H](Nc2cc(C(F)(F)F)nc3ccc(OC(F)(F)F)cc23)C1] |
| 133 | 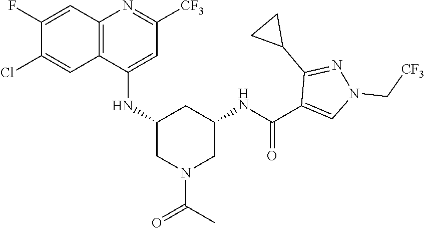 [SMILES: CC(=O)N1C[C@@H](NC(=O)c2cn(CC(F)(F)F)nc2C2CC2)C[C@@H](Nc2cc(C(F)(F)F)nc3cc(F)c(Cl)cc23)C1] |
| 134 | 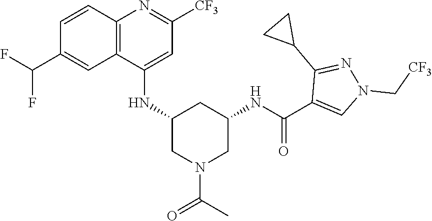 [SMILES: CC(=O)N1C[C@@H](NC(=O)c2cn(CC(F)(F)F)nc2C2CC2)C[C@@H](Nc2cc(C(F)(F)F)nc3ccc(C(F)F)cc23)C1] |
| 135 | 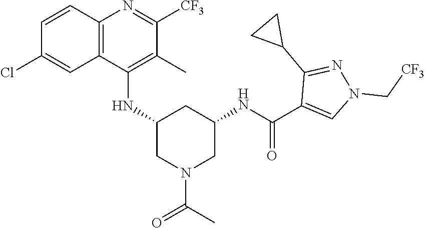 [SMILES: CC(=O)N1C[C@@H](NC(=O)c2cn(CC(F)(F)F)nc2C2CC2)C[C@@H](Nc2c(C)c(C(F)(F)F)nc3ccc(Cl)cc23)C1] |
| 136 | 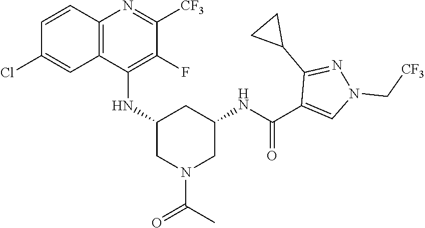 [SMILES: CC(=O)N1C[C@@H](NC(=O)c2cn(CC(F)(F)F)nc2C2CC2)C[C@@H](Nc2c(F)c(C(F)(F)F)nc3ccc(Cl)cc23)C1] |
| 137 | 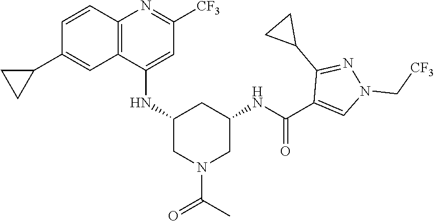 [SMILES: CC(=O)N1C[C@@H](NC(=O)c2cn(CC(F)(F)F)nc2C2CC2)C[C@@H](Nc2cc(C(F)(F)F)nc3ccc(C4CC4)cc23)C1] |
| 138 | 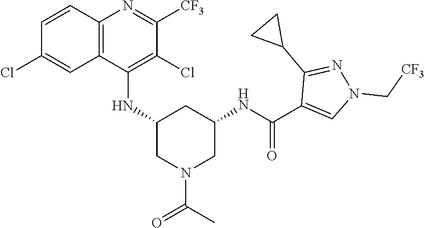 [SMILES: CC(=O)N1C[C@@H](NC(=O)c2cn(CC(F)(F)F)nc2C2CC2)C[C@@H](Nc2c(Cl)c(C(F)(F)F)nc3ccc(Cl)cc23)C1] |
| 139 | 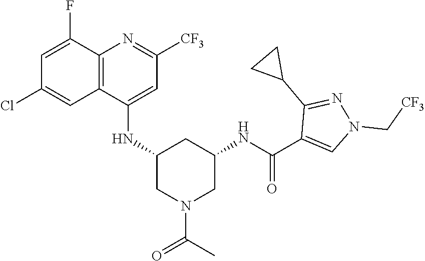 [SMILES: CC(=O)N1C[C@@H](NC(=O)c2cn(CC(F)(F)F)nc2C2CC2)C[C@@H](Nc2cc(C(F)(F)F)nc3c(F)cc(Cl)cc23)C1] |
| 140 | 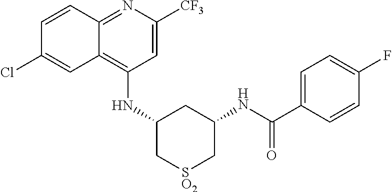 [SMILES: O=C(N[C@H]1C[C@@H](Nc2cc(C(F)(F)F)nc3ccc(Cl)cc23)CS(=O)(=O)C1)c1ccc(F)cc1] |

10. A pharmaceutical composition comprising the compound of claim 1 and a pharmaceutically acceptable carrier.

11. A method for treating a disease, disorder, or condition where modulation of a MRGPR is implicated, wherein the method comprises administering to a system or subject in need of such treatment an effective amount of the compound of claim 1, wherein the MRGPR is MRGPR X2.

12. The method of claim 11, wherein the disease, disorder or condition is a pseudo-allergic reaction, an itch associated condition, a pain associated condition, an inflammatory or autoimmune disorder.

13. The method of claim 12, wherein the itch associated condition is chronic itch; contact dermatitis; Allergic blepharitis; Anemia; Atopic dermatitis; Bullous pemphigoid; Candidiasis; Chicken pox; end-stage renal failure; hemodialysis; Chronic urticaria; Contact dermatitis, Atopic Dermatitis; Dermatitis herpetiformis; Diabetes; Drug allergy, Dry skin; Dyshidrotic dermatitis; Ectopic eczema; Eosinophilic fasciitis; Epidermolysis bullosa; Erythrasma; Food allergy; Folliculitis; Fungal skin infection; Hemorrhoids; Herpes; HIV infection; Hodgkin's disease; Hyperthyroidism; Iodinated contrast dye allergy; Iron deficiency anemia; Kidney disease; Leukemia, porphyrias; Lymphoma; Malignancy; Mastocystosis; Multiple myeloma; Neurodermatitis; Onchocerciasis; Paget's disease; Pediculosis; Polycythemia rubra vera; Prurigo nodularis; Lichen Planus; Lichen Sclerosis; Pruritus ani; Pseudorabies; Psoriasis; Rectal prolapse; Sarcoidosis granulomas; Scabies; Schistosomiasis; Scleroderma, Severe stress, Stasia dermatitis; Swimmer's itch; Thyroid disease; Tinea cruris; Rosacea; Cutaneous amyloidosis; Scleroderma; Acne; wound healing; burn healing; ocular itch; or Urticaria.

14. The method of claim 12, wherein the itch associated condition is urticaria, pruritus, atopic dermatitis, dry skin, psoriasis, contact dermatitis, or eczema.

15. The method of claim 12, wherein the pain associated condition is Acute Pain, Advanced Prostate Cancer, AIDS-Related Pain, Ankylosing Spondylitis, Arachnoiditis, Arthritis, Arthrofibrosis, Ataxic Cerebral Palsy, Autoimmune Atrophic Gastritis, Avascular Necrosis, Back Pain, Behcet's Disease (Syndrome), Burning Mouth Syndrome, Bursitis, Cancer Pain, Carpal Tunnel, Cauda Equina Syndrome, Central Pain Syndrome, Cerebral Palsy, Cervical Stenosis, Charcot-Marie-Tooth (CMT) Disease, Chronic Fatigue Syndrome (CFS), Chronic Functional Abdominal Pain (CFAP), Chronic Pain, Chronic Pancreatitis, Chronic Pelvic Pain Syndrome, Collapsed Lung (Pneumothorax), Complex Regional Pain Syndrome (RSD), Corneal Neuropathic Pain, Crohn's Disease, Degenerative Disc Disease, Dental Pain, Dercum's Disease, Dermatomyositis, Diabetic Peripheral Neuropathy (DPN), Dystonia, Ehlers-Danlos Syndrome (EDS), Endometriosis, Eosinophilia-Myalgia Syndrome (EMS), Erythromelalgia, Fibromyalgia, Gout, Headaches, Herniated disc, Hydrocephalus, Intercostal neuralgia, Interstitial Cystitis, Irritable Bowel syndrome (IBS), Juvenile Dermatositis (Dermatomyositis), Knee Injury, Leg Pain, Loin Pain-Haematuria Syndrome, Lupus, Lyme Disease, Medullary Sponge Kidney (MSK), Meralgia Paresthetica, Mesothelioma, Migraine, Musculoskeletal pain, Myofascial Pain, Myositis, Neck Pain, Neuropathic Pain, Occipital Neuralgia, Osteoarthritis, Paget's Disease, Parsonage Turner Syndrome, Pelvic Pain, Periodontitis Pain, Peripheral Neuropathy, Phantom Limb Pain, Pinched Nerve, Polycystic Kidney Disease, Polymyalgia Rhuematica, Polymyositis, Porphyria, Post Herniorrhaphy Pain Syndrome, Post Mastectomy, Postoperative Pain, Pain Syndrome, Post Stroke Pain, Post Thorocotomy Pain Syndrome, Postherpetic Neuralgia (Shingles), Post-Polio Syndrome, Primary Lateral Sclerosis, Psoriatic Arthritis, Pudendal Neuralgia, Radiculopathy, Raynaud's Disease, Rheumatoid Arthritis (RA), Sacroiliac Joint Dysfunction, sarcoidosis, Scheuermann's Kyphosis Disease, Sciatica, Scoliosis, Shingles (Herpes Zoster), Sjogren's Syndrome, Spasmodic Torticollis, Sphincter of Oddi Dysfunction, Spinal Cerebellum Ataxia (SCA Ataxia), Spinal Cord Injury, Spinal Stenosis, Syringomyelia, Tarlov Cysts, Transverse Myelitis, Trigeminal Neuralgia, Neuropathic Pain, Ulcerative Colitis, Vascular Pain or Vulvodynia.

16. The method of claim 12, wherein the inflammatory or autoimmune disorder is chronic inflammation, mast cell activation syndrome, Multiple Sclerosis, Steven Johnson's Syndrome, Toxic Epidermal Necrolysis, appendicitis, bursitis, cutaneous lupus, colitis, cystitis, dermatitis, phlebitis, reflex sympathetic dystrophy/complex regional pain syndrome (rsd/crps), rhinitis, tendonitis, tonsillitis, acne vulgaris, sinusitis, rosacea, psoriasis, graft-versus-host disease, reactive airway disorder, asthma, airway infection, autoinflammatory disease, celiac disease, chronic prostatitis, diverticulitis, glomerulonephritis, hidradenitis suppurativa, hypersensitivities, intestinal disorder, epithelial intestinal disorder, inflammatory bowel disease, irritable bowel syndrome, Crohn's Disease, ulcerative colitis, lupus erythematous, interstitial cystitis, otitis, pelvic inflammatory disease, endometrial pain, reperfusion injury, rheumatic fever, rheumatoid arthritis, sarcoidosis, transplant rejection, psoriasis, lung inflammation, chronic obstructive pulmonary disease, cardiovascular disease, or vasculitis.

17-20. (canceled)
